SEQUENCE LISTING (391 sequences)

SEQ ID NO: 1 - RNA, 22 nt, Homo sapiens
aauugcacgguauccaucugua

SEQ ID NO: 2 - RNA, 21 nt, Homo sapiens
cuagacugaagcuccuugagg

SEQ ID NO: 3 - RNA, 23 nt, Homo sapiens
caaagugcuuacagugcagguag

SEQ ID NO: 4 - RNA, 23 nt, Homo sapiens
caaagugcucauagugcagguag

SEQ ID NO: 5 - RNA, 23 nt, Homo sapiens
agcagcauuguacagggcuauga

SEQ ID NO: 6 - RNA, 23 nt, Homo sapiens
aaaagugcuuacagugcagguag

SEQ ID NO: 7 - RNA, 21 nt, Homo sapiens
uacaguacugugauaacugaa

SEQ ID NO: 8 - RNA, 23 nt, Homo sapiens
uaaagugcuuauagugcagguag

SEQ ID NO: 9 - RNA, 22 nt, Homo sapiens
agagguaguagguugcauaguu

SEQ ID NO: 10 - RNA, 19 nt, Homo sapiens
aaaagcuggguugagagga

SEQ ID NO: 11 - RNA, 20 nt, Homo sapiens
acugccccaggugcugcugg

SEQ ID NO: 12 - RNA, 22 nt, Homo sapiens
uagcagcacaucaugguuuaca

SEQ ID NO: 13 - RNA, 22 nt, Homo sapiens
ucagugcaucacagaacuuugu

SEQ ID NO: 14 - RNA, 22 nt, Homo sapiens
ugucuuacucccucaggcacau

SEQ ID NO: 15 - RNA, 22 nt, Homo sapiens
uguaaacauccccgacuggaag

SEQ ID NO: 16 - RNA, 22 nt, Homo sapiens
uucaaguaauccaggauaggcu

SEQ ID NO: 17 - RNA, 23 nt, Homo sapiens
agcagcauuguacagggcuauca

SEQ ID NO: 18 - RNA, 22 nt, Homo sapiens
aaaagcuggguugagagggcaa

SEQ ID NO: 19 - RNA, 22 nt, Homo sapiens
uggcucaguucagcaggaacag

SEQ ID NO: 20 - RNA, 23 nt, Homo sapiens
uaaggugcaucuagugcagauag

SEQ ID NO: 21 - RNA, 20 nt, Homo sapiens
aaaagcuggguugagagggu

SEQ ID NO: 22 - RNA, 22 nt, Homo sapiens
ugcggggcuagggcuaacagca

SEQ ID NO: 23 - RNA, 23 nt, Homo sapiens
caaagugcuguucgugcagguag

SEQ ID NO: 24 - RNA, 23 nt, Homo sapiens
uguaaacauccuacacucucagc

SEQ ID NO: 25 - RNA, 21 nt, Homo sapiens
uaaagugcugacagugcagau

SEQ ID NO: 26 - RNA, 24 nt, Homo sapiens
uuuggcaaugguagaacucacacu

SEQ ID NO: 27 - RNA, 22 nt, Homo sapiens
aaaagcuggguugagagggcga

SEQ ID NO: 28 - RNA, 17 nt, Homo sapiens
ucucgcuggggccucca

SEQ ID NO: 29 - RNA, 22 nt, Homo sapiens
uguaaacauccuugacuggaag

SEQ ID NO: 30 - RNA, 23 nt, Homo sapiens
uauucauuuauccccagccuaca

SEQ ID NO: 31 - RNA, 22 nt, Homo sapiens
uagcagcacguaaauauuggcg

SEQ ID NO: 32 - RNA, 22 nt, Homo sapiens
uacccauugcauaucggaguug

SEQ ID NO: 33 - RNA, 22 nt, Homo sapiens
cagugcaauguuaaaagggcau

SEQ ID NO: 34 - RNA, 22 nt, Homo sapiens
ucagugcacuacagaacuuugu

SEQ ID NO: 35 - RNA, 22 nt, Homo sapiens
acugcugagcuagcacuucccg

SEQ ID NO: 36 - RNA, 23 nt, Homo sapiens
ugaggggcagagagcgagacuuu

SEQ ID NO: 37 - RNA, 22 nt, Homo sapiens
acugcagugaaggcacuuguag

SEQ ID NO: 38 - RNA, 22 nt, Homo sapiens
ugagguaguagguuguauaguu

SEQ ID NO: 39 - RNA, 21 nt, Homo sapiens
gccccugggccuauccuagaa

SEQ ID NO: 40 - RNA, 21 nt, Homo sapiens
cugaccuaugaauugacagcc

SEQ ID NO: 41 - RNA, 23 nt, Homo sapiens
uagcaccauuugaaaucaguguu

SEQ ID NO: 42 - RNA, 22 nt, Homo sapiens
uguaaacauccucgacuggaag

SEQ ID NO: 43 - RNA, 22 nt, Homo sapiens
aaggagcucacagucuauugag

SEQ ID NO: 44 - RNA, 21 nt, Homo sapiens
cggcggggacggcgauugguc

SEQ ID NO: 45 - RNA, 18 nt, Homo sapiens
aucccaccucugccacca

SEQ ID NO: 46 - RNA, 22 nt, Homo sapiens
uucaccaccuucuccacccagc

SEQ ID NO: 47 - RNA, 22 nt, Homo sapiens
ccucccacacccaaggcuugca

SEQ ID NO: 48 - RNA, 23 nt, Homo sapiens
uaauccuugcuaccugggugaga

SEQ ID NO: 49 - RNA, 22 nt, Homo sapiens
ugucaguuugucaaauacccca

SEQ ID NO: 50 - RNA, 21 nt, Homo sapiens
uagcagcacagaaauauuggc

SEQ ID NO: 51 - RNA, 23 nt, Homo sapiens
ucucacacagaaaucgcacccgu

SEQ ID NO: 52 - RNA, 22 nt, Homo sapiens
cauugcacuugucucggucuga

SEQ ID NO: 53 - RNA, 21 nt, Homo sapiens
agcuacaucuggcuacugggu

SEQ ID NO: 54 - RNA, 17 nt, Homo sapiens
ucccaccgcugccaccc

SEQ ID NO: 55 - RNA, 23 nt, Homo sapiens
aacauucaacgcugucggugagu

SEQ ID NO: 56 - RNA, 22 nt, Homo sapiens
uguaacagcaacuccaugugga

SEQ ID NO: 57 - RNA, 23 nt, Homo sapiens
cccaguguucagacuaccuguuc

SEQ ID NO: 58 - RNA, 22 nt, Homo sapiens
ucccugagacccuaacuuguga

SEQ ID NO: 59 - RNA, 23 nt, Homo sapiens
ucccuguccuccaggagcucacg

SEQ ID NO: 60 - RNA, 22 nt, Homo sapiens
ucaggcucaguccccucccgau

SEQ ID NO: 61 - RNA, 22 nt, Homo sapiens
cacgcucaugcacacacccaca

SEQ ID NO: 62 - RNA, 22 nt, Homo sapiens
uagcaccaucugaaaucgguua

SEQ ID NO: 63 - RNA, 22 nt, Homo sapiens
uagcagcacauaaugguuugug

SEQ ID NO: 64 - RNA, 21 nt, Homo sapiens
ucgaggagcucacagucuagu

SEQ ID NO: 65 - RNA, 23 nt, Homo sapiens
ugagugugugugugugagugugu

SEQ ID NO: 66 - RNA, 22 nt, Homo sapiens
ucugcccccuccgcugcugcca

SEQ ID NO: 67 - RNA, 23 nt, Homo sapiens
aaugacacgaucacucccguuga

SEQ ID NO: 68 - RNA, 20 nt, Homo sapiens
uacaguauagaugauguacu

SEQ ID NO: 69 - RNA, 22 nt, Homo sapiens
ucguaccgugaguaauaaugcg

SEQ ID NO: 70 - RNA, 22 nt, Homo sapiens
ggauaucaucauauacuguaag

SEQ ID NO: 71 - RNA, 22 nt, Homo sapiens
auauaauacaaccugcuaagug

SEQ ID NO: 72 - RNA, 22 nt, Homo sapiens
uagcuuaucagacugauguuga

SEQ ID NO: 73 - RNA, 23 nt, Homo sapiens
cagugcaauaguauugucaaagc

SEQ ID NO: 74 - RNA, 22 nt, Homo sapiens
ugagguaguaguuugugcuguu

SEQ ID NO: 75 - RNA, 22 nt, Homo sapiens
ugagguaguagauuguauaguu

SEQ ID NO: 76 - RNA, 23 nt, Homo sapiens
acugcccuaagugcuccuucugg

SEQ ID NO: 77 - RNA, 22 nt, Homo sapiens
uauggcacugguagaauucacu

SEQ ID NO: 78 - RNA, 21 nt, Homo sapiens
gugccagcugcagugggggag

SEQ ID NO: 79 - RNA, 23 nt, Homo sapiens
uagcagcgggaacaguucugcag

SEQ ID NO: 80 - RNA, 25 nt, Homo sapiens
aggcaccagccaggcauugcucagc

SEQ ID NO: 81 - RNA, 19 nt, Homo sapiens
aagcagcugccucugaggc

SEQ ID NO: 82 - RNA, 22 nt, Homo sapiens
aacccguagauccgaacuugug

SEQ ID NO: 83 - RNA, 24 nt, Homo sapiens
ucccugagacccuuuaaccuguga

SEQ ID NO: 84 - RNA, 23 nt, Homo sapiens
guccaguuuucccaggaaucccu

SEQ ID NO: 85 - RNA, 22 nt, Homo sapiens
cugcgcaagcuacugccuugcu

SEQ ID NO: 86 - RNA, 22 nt, Homo sapiens
cagcagcaauucauguuuugaa

SEQ ID NO: 87 - RNA, 22 nt, Homo sapiens
aacauucaaccugucggugagu

SEQ ID NO: 88 - RNA, 21 nt, Homo sapiens
ucagugcaugacagaacuugg

SEQ ID NO: 89 - RNA, 22 nt, Homo sapiens
acagcaggcacagacaggcagu

SEQ ID NO: 90 - RNA, 24 nt, Homo sapiens
aauccuuggaaccuaggugugagu

SEQ ID NO: 91 - RNA, 22 nt, Homo sapiens
caugccuugaguguaggaccgu

SEQ ID NO: 92 - RNA, 26 nt, Homo sapiens
gugagggcaugcaggccuggaugggg

SEQ ID NO: 93 - RNA, 23 nt, Homo sapiens
ccugcagcgacuugauggcuucc

SEQ ID NO: 94 - RNA, 22 nt, Homo sapiens
ugcuaugccaacauauugccau

SEQ ID NO: 95 - RNA, 22 nt, Homo sapiens
uacccagagcaugcagugugaa

SEQ ID NO: 96 - RNA, 21 nt, Homo sapiens
cccggagccaggaugcagcuc

SEQ ID NO: 97 - RNA, 22 nt, Homo sapiens
ugagccccugugccgcccccag

SEQ ID NO: 98 - RNA, 22 nt, Homo sapiens
ccucuagauggaagcacugucu

SEQ ID NO: 99 - RNA, 24 nt, Homo sapiens
uucuccaaaagaaagcacuuucug

SEQ ID NO: 100 - RNA, 21 nt, Homo sapiens
ugucuugcaggccgucaugca

SEQ ID NO: 101 - RNA, 21 nt, Homo sapiens
cucccacaugcaggguuugca

SEQ ID NO: 102 - RNA, 23 nt, Homo sapiens
uacugcaucaggaacugauugga

SEQ ID NO: 103 - RNA, 21 nt, Homo sapiens
aaggcagggcccccgcucccc

SEQ ID NO: 104 - RNA, 27 nt, Homo sapiens
auugaucaucgacacuucgaacgcaau

SEQ ID NO: 105 - RNA, 21 nt, Homo sapiens
uucacaguggcuaaguucugc

SEQ ID NO: 106 - RNA, 21 nt, Homo sapiens
uucaaguaauucaggauaggu

SEQ ID NO: 107 - RNA, 21 nt, Homo sapiens
uucacaguggcuaaguuccgc

SEQ ID NO: 108 - RNA, 22 nt, Homo sapiens
ugagguaguaguuuguacaguu

SEQ ID NO: 109 - RNA, 22 nt, Homo sapiens
uuauaauacaaccugauaagug

SEQ ID NO: 110 - RNA, 24 nt, Homo sapiens
uugcagcugccugggagugacuuc

SEQ ID NO: 111 - RNA, 22 nt, Homo sapiens
uacugcagacguggcaaucaug

SEQ ID NO: 112 - RNA, 22 nt, Homo sapiens
uuaugguuugccugggacugag

SEQ ID NO: 113 - RNA, 21 nt, Homo sapiens
cggggcagcucaguacaggau

SEQ ID NO: 114 - RNA, 23 nt, Homo sapiens
uuuggcacuagcacauuuuugcu

SEQ ID NO: 115 - RNA, 26 nt, Homo sapiens
gaugaugauggcagcaaauucugaaa

SEQ ID NO: 116 - RNA, 21 nt, Homo sapiens
ugagaugaagcacuguagcuc

SEQ ID NO: 117 - RNA, 21 nt, Homo sapiens
uuggccacaauggguuagaac

SEQ ID NO: 118 - RNA, 22 nt, Homo sapiens
ugccuacugagcugaaacacag

SEQ ID NO: 119 - RNA, 22 nt, Homo sapiens
uggugggcacagaaucuggacu

SEQ ID NO: 120 - RNA, 24 nt, Homo sapiens
agccuggaagcuggagccugcagu

SEQ ID NO: 121 - RNA, 21 nt, Homo sapiens
ucuaguaagaguggcagucga

SEQ ID NO: 122 - RNA, 22 nt, Homo sapiens
ggugcagugcugcaucucuggu

SEQ ID NO: 123 - RNA, 22 nt, Homo sapiens
uggugggccgcagaacaugugc

SEQ ID NO: 124 - RNA, 21 nt, Homo sapiens
ugcaggaccaagaugagcccu

SEQ ID NO: 125 - RNA, 23 nt, Homo sapiens
uaaggugcaucuagugcaguuag

SEQ ID NO: 126 - RNA, 23 nt, Homo sapiens
agugccugagggaguaagagccc

SEQ ID NO: 127 - RNA, 22 nt, Homo sapiens
cuauacgaccugcugccuuucu

SEQ ID NO: 128 - RNA, 22 nt, Homo sapiens
acacagggcuguugugaagacu

SEQ ID NO: 129 - RNA, 20 nt, Homo sapiens
gugcauugcuguugcauugc

SEQ ID NO: 130 - RNA, 19 nt, Homo sapiens
gagccaguuggacaggagc

SEQ ID NO: 131 - RNA, 21 nt, Homo sapiens
aggcaagaugcuggcauagcu

SEQ ID NO: 132 - RNA, 21 nt, Homo sapiens
gcaguccaugggcauauacac

SEQ ID NO: 133 - RNA, 22 nt, Homo sapiens
ucuacaaaggaaagcgcuuucu

SEQ ID NO: 134 - RNA, 22 nt, Homo sapiens
ggaggggucccgcacugggagg

SEQ ID NO: 135 - RNA, 22 nt, Homo sapiens
cggcaacaagaaacugccugag

SEQ ID NO: 136 - RNA, 21 nt, Homo sapiens
gaagugugccguggugugucu

SEQ ID NO: 137 - RNA, 22 nt, Homo sapiens
cugccaauuccauaggucacag

SEQ ID NO: 138 - RNA, 22 nt, Homo sapiens
ccaauauuacugugcugcuuua

SEQ ID NO: 139 - RNA, 22 nt, Homo sapiens
aaucaugugcagugccaauaug

SEQ ID NO: 140 - RNA, 21 nt, Homo sapiens
gugcauuguaguugcauugca

SEQ ID NO: 141 - RNA, 21 nt, Homo sapiens
aggaggcagcgcucucaggac

SEQ ID NO: 142 - RNA, 21 nt, Homo sapiens
uuaggccgcagaucuggguga

SEQ ID NO: 143 - RNA, 23 nt, Homo sapiens
ccucagggcuguagaacagggcu

SEQ ID NO: 144 - RNA, 22 nt, Homo sapiens
uauugcacauuacuaaguugca

SEQ ID NO: 145 - RNA, 22 nt, Homo sapiens
uccagcaucagugauuuuguug

SEQ ID NO: 146 - RNA, 21 nt, Homo sapiens
acucuagcugccaaaggcgcu

SEQ ID NO: 147 - RNA, 22 nt, Homo sapiens
cuggcccucucugcccuuccgu

SEQ ID NO: 148 - RNA, 22 nt, Homo sapiens
cagugccucggcagugcagccc

SEQ ID NO: 149 - RNA, 21 nt, Homo sapiens
agaccuggcccagaccucagc

SEQ ID NO: 150 - RNA, 22 nt, Homo sapiens
cucuagagggaagcgcuuucug

SEQ ID NO: 151 - RNA, 22 nt, Homo sapiens
ggcuacaacacaggacccgggc

SEQ ID NO: 152 - RNA, 22 nt, Homo sapiens
uaacagucuacagccauggucg

SEQ ID NO: 153 - RNA, 22 nt, Homo sapiens
ugagaacugaauuccauaggcu

SEQ ID NO: 154 - RNA, 23 nt, Homo sapiens
uagugcaauauugcuuauagggu

SEQ ID NO: 155 - RNA, 24 nt, Homo sapiens
agcagaagcagggagguucuccca

SEQ ID NO: 156 - RNA, 22 nt, Homo sapiens
cucuagagggaagcgcuuucug

SEQ ID NO: 157 - RNA, 22 nt, Homo sapiens
caggccauauugugcugccuca

SEQ ID NO: 158 - RNA, 22 nt, Homo sapiens
ugagguaggagguuguauaguu

SEQ ID NO: 159 - RNA, 22 nt, Homo sapiens
acaguagucugcacauugguua

SEQ ID NO: 160 - RNA, 21 nt, Homo sapiens
cuccagagggaugcacuuucu

SEQ ID NO: 161 - RNA, 18 nt, Homo sapiens
ugcuuccuuucagagggu

SEQ ID NO: 162 - RNA, 22 nt, Homo sapiens
acaguagucugcacauugguua

SEQ ID NO: 163 - RNA, 22 nt, Homo sapiens
ugucuacuacuggagacacugg

SEQ ID NO: 164 - RNA, 22 nt, Homo sapiens
uuuuucauuauugcuccugacc

SEQ ID NO: 165 - RNA, 22 nt, Homo sapiens
cgucaacacuugcugguuuccu

SEQ ID NO: 166 - RNA, 22 nt, Homo sapiens
uccauuacacuacccugccucu

SEQ ID NO: 167 - RNA, 22 nt, Homo sapiens
aagcccuuaccccaaaaaguau

SEQ ID NO: 168 - RNA, 22 nt, Homo sapiens
aagugcugucauagcugagguc

SEQ ID NO: 169 - RNA, 22 nt, Homo sapiens
ggagacgcggcccuguuggagu

SEQ ID NO: 170 - RNA, 19 nt, Homo sapiens
aggcugcggaauucaggac

SEQ ID NO: 171 - RNA, 22 nt, Homo sapiens
ugaccgauuucuccugguguuc

SEQ ID NO: 172 - RNA, 23 nt, Homo sapiens
uaagugcuuccauguuuuaguag

SEQ ID NO: 173 - RNA, 22 nt, Homo sapiens
cguguucacagcggaccuugau

SEQ ID NO: 174 - RNA, 22 nt, Homo sapiens
caaaaaucucaauuacuuuugc

SEQ ID NO: 175 - RNA, 22 nt, Homo sapiens
cgucuuacccagcaguguuugg

SEQ ID NO: 176 - RNA, 20 nt, Homo sapiens
caccaggcauuguggucucc

SEQ ID NO: 177 - RNA, 22 nt, Homo sapiens
aucgugcauccuuuuagagugu

SEQ ID NO: 178 - RNA, 22 nt, Homo sapiens
augggugaauuuguagaaggau

SEQ ID NO: 179 - RNA, 22 nt, Homo sapiens
aaaacuguaauuacuuuuguac

SEQ ID NO: 180 - RNA, 21 nt, Homo sapiens
auccuugcuaucugggugcua

SEQ ID NO: 181 - RNA, 22 nt, Homo sapiens
caaccuggaggacuccaugcug

SEQ ID NO: 182 - RNA, 21 nt, Homo sapiens
ucacuccucuccucccgucuu

SEQ ID NO: 183 - RNA, 22 nt, Homo sapiens
ucgugucuuguguugcagccgg

SEQ ID NO: 184 - RNA, 22 nt, Homo sapiens
aaagugcauccuuuuagagguu

SEQ ID NO: 185 - RNA, 22 nt, Homo sapiens
uagaguuacacccugggaguua

SEQ ID NO: 186 - RNA, 22 nt, Homo sapiens
aaaccuguguuguucaagaguc

SEQ ID NO: 187 - RNA, 22 nt, Homo sapiens
aucgugcaucccuuuagagugu

SEQ ID NO: 188 - RNA, 22 nt, Homo sapiens
aguauucuguaccagggaaggu

SEQ ID NO: 189 - RNA, 21 nt, Homo sapiens
cauuauuacuuuugguacgcg

SEQ ID NO: 190 - RNA, 21 nt, Homo sapiens
aauauuauacagucaaccucu

SEQ ID NO: 191 - RNA, 21 nt, Homo sapiens
aguuaaugaauccuggaaagu

SEQ ID NO: 192 - RNA, 19 nt, Homo sapiens
gugucugcuuccuguggga

SEQ ID NO: 193 - RNA, 22 nt, Homo sapiens
cuccugacuccagguccugugu

SEQ ID NO: 194 - RNA, 21 nt, Homo sapiens
aaaacggugagauuuuguuuu

SEQ ID NO: 195 - RNA, 23 nt, Homo sapiens
uuauugcuuaagaauacgcguag

SEQ ID NO: 196 - RNA, 27 nt, Homo sapiens
cacuguaggugauggugagagugggca

SEQ ID NO: 197 - RNA, 22 nt, Homo sapiens
auauuaccauuagcucaucuuu

SEQ ID NO: 198 - RNA, 21 nt, Homo sapiens
ugauauguuugauauuggguu

SEQ ID NO: 199 - RNA, 22 nt, Homo sapiens
accuugccuugcugcccgggcc

SEQ ID NO: 200 - RNA, 22 nt, Homo sapiens
caaagaauucuccuuuugggcu

SEQ ID NO: 201 - RNA, 21 nt, Homo sapiens
cuccguuugccuguuucgcug

SEQ ID NO: 202 - RNA, 22 nt, Homo sapiens
acccuaucaauauugucucugc

SEQ ID NO: 203 - RNA, 22 nt, Homo sapiens
uuuucaacucuaaugggagaga

SEQ ID NO: 204 - RNA, 22 nt, Homo sapiens
agacuucccauuugaagguggc

SEQ ID NO: 205 - RNA, 22 nt, Homo sapiens
gaaaucaagcgugggugagacc

SEQ ID NO: 206 - RNA, 21 nt, Homo sapiens
uguucauguagauguuuaagc

SEQ ID NO: 207 - RNA, 21 nt, Homo sapiens
ugguagacuauggaacguagg

SEQ ID NO: 208 - RNA, 22 nt, Homo sapiens
aaagugcuuccuuuuagagggu

SEQ ID NO: 209 - RNA, 22 nt, Homo sapiens
aaaaguaauugugguuuuggcc

SEQ ID NO: 210 - RNA, 22 nt, Homo sapiens
aaagugcauccuuuuagagugu

SEQ ID NO: 211 - RNA, 23 nt, Homo sapiens
uaaaucccauggugccuucuccu

SEQ ID NO: 212 - RNA, 21 nt, Homo sapiens
cacauuacacggucgaccucu

SEQ ID NO: 213 - RNA, 21 nt, Homo sapiens
aguuaggauuaggucguggaa

SEQ ID NO: 214 - RNA, 22 nt, Homo sapiens
ugcaacuuaccugagucauuga

SEQ ID NO: 215 - RNA, 22 nt, Homo sapiens
gaauguugcucggugaaccccu

SEQ ID NO: 216 - RNA, 21 nt, Homo sapiens
acucuuucccuguugcacuac

SEQ ID NO: 217 - RNA, 22 nt, Homo sapiens
aaaguucugagacacuccgacu

SEQ ID NO: 218 - RNA, 25 nt, Homo sapiens
aaaggauucugcugucggucccacu

SEQ ID NO: 219 - RNA, 19 nt, Homo sapiens
uggauuuuuggaucaggga

SEQ ID NO: 220 - RNA, 21 nt, Homo sapiens
uaaaguaaauaugcaccaaaa

SEQ ID NO: 221 - RNA, 22 nt, Homo sapiens
agggcuuagcugcuugugagca

SEQ ID NO: 222 - RNA, 20 nt, Homo sapiens
uaaagagcccuguggagaca

SEQ ID NO: 223 - RNA, 19 nt, Homo sapiens
agguugacauacguuuccc

SEQ ID NO: 224 - RNA, 22 nt, Homo sapiens
uccgagccugggucucccucuu

SEQ ID NO: 225 - RNA, 22 nt, Homo sapiens
auaagacgaacaaaagguuugu

SEQ ID NO: 226 - RNA, 23 nt, Homo sapiens
agguuacccgagcaacuuugcau

SEQ ID NO: 227 - RNA, 21 nt, Homo sapiens
aauaauacaugguugaucuuu

SEQ ID NO: 228 - RNA, 21 nt, Homo sapiens
cgcgggugcuuacugacccuu

SEQ ID NO: 229 - RNA, 22 nt, Homo sapiens
aauccuuugucccugggugaga

SEQ ID NO: 230 - RNA, 22 nt, Homo sapiens
uuugugaccugguccacuaacc

SEQ ID NO: 231 - RNA, 23 nt, Homo sapiens
ucuuugguuaucuagcuguauga

SEQ ID NO: 232 - RNA, 21 nt, Homo sapiens
agggagggacgggggcugugc

SEQ ID NO: 233 - RNA, 22 nt, Homo sapiens
cagugcaaugaugaaagggcau

SEQ ID NO: 234 - RNA, 22 nt, Homo sapiens
uguaaacauccuacacucagcu

SEQ ID NO: 235 - RNA, 22 nt, Homo sapiens
cuuggcaccuagcaagcacuca

SEQ ID NO: 236 - RNA, 17 nt, Homo sapiens
ucccuguucgggcgcca

SEQ ID NO: 237 - RNA, 23 nt, Homo sapiens
ugugcaaauccaugcaaaacuga

SEQ ID NO: 238 - RNA, 22 nt, Homo sapiens
uggagagaaaggcaguuccuga

SEQ ID NO: 239 - RNA, 21 nt, Homo sapiens
cauaaaguagaaagcacuacu

SEQ ID NO: 240 - RNA, 23 nt, Homo sapiens
uggugcggagagggcccacagug

SEQ ID NO: 241 - RNA, 22 nt, Homo sapiens
cugugcgugugacagcggcuga

SEQ ID NO: 242 - RNA, 22 nt, Homo sapiens
cucggcgcggggcgcgggcucc

SEQ ID NO: 243 - RNA, 22 nt, Homo sapiens
gugagucucuaagaaaagagga

SEQ ID NO: 244 - RNA, 21 nt, Homo sapiens
aucacauugccagggauuucc

SEQ ID NO: 245 - RNA, 22 nt, Homo sapiens
ucuacagugcacgugucuccag

SEQ ID NO: 246 - RNA, 22 nt, Homo sapiens
uccuguacugagcugccccgag

SEQ ID NO: 247 - RNA, 21 nt, Homo sapiens
agggggaaaguucuauagucc

SEQ ID NO: 248 - RNA, 21 nt, Homo sapiens
ccccaccuccucucuccucag

SEQ ID NO: 249 - RNA, 22 nt, Homo sapiens
ugggucuuugcgggcgagauga

SEQ ID NO: 250 - RNA, 21 nt, Homo sapiens
aggggugcuaucugugauuga

SEQ ID NO: 251 - RNA, 22 nt, Homo sapiens
cuugguucagggagggucccca

SEQ ID NO: 252 - RNA, 20 nt, Homo sapiens
guagaggagauggcgcaggg

SEQ ID NO: 253 - RNA, 21 nt, Homo sapiens
caacaccagucgaugggcugu

SEQ ID NO: 254 - RNA, 17 nt, Homo sapiens
ucgccuccuccucuccc

SEQ ID NO: 255 - RNA, 22 nt, Homo sapiens
agaucgaccguguuauauucgc

SEQ ID NO: 256 - RNA, 21 nt, Homo sapiens
ugguucuagacuugccaacua

SEQ ID NO: 257 - RNA, 23 nt, Homo sapiens
acuccauuuguuuugaugaugga

SEQ ID NO: 258 - RNA, 17 nt, Homo sapiens
ucauauugcuucuuucu

SEQ ID NO: 259 - RNA, 21 nt, Homo sapiens
aaagugcuuccuuuuugaggg

SEQ ID NO: 260 - RNA, 22 nt, Homo sapiens
cuauacaaccuacugccuuccc

SEQ ID NO: 261 - RNA, 22 nt, Homo sapiens
aaucauacacgguugaccuauu

SEQ ID NO: 262 - RNA, 22 nt, Homo sapiens
uugggaucauuuugcauccaua

SEQ ID NO: 263 - RNA, 20 nt, Homo sapiens
aaaguagcuguaccauuugc

SEQ ID NO: 264 - RNA, 22 nt, Homo sapiens
ugaaggucuacugugugccagg

SEQ ID NO: 265 - RNA, 21 nt, Homo sapiens
caaagguauuugugguuuuug

SEQ ID NO: 266 - RNA, 23 nt, Homo sapiens
caagucuuauuugagcaccuguu

SEQ ID NO: 267 - RNA, 23 nt, Homo sapiens
guuugcacgggugggccuugucu

SEQ ID NO: 268 - RNA, 23 nt, Homo sapiens
ugauuguagccuuuuggaguaga

SEQ ID NO: 269 - RNA, 23 nt, Homo sapiens
ccaguuaccgcuuccgcuaccgc

SEQ ID NO: 270 - RNA, 22 nt, Homo sapiens
cuauacagucuacugucuuucc

SEQ ID NO: 271 - RNA, 22 nt, Homo sapiens
gaaagugcuuccuuuuagaggc

SEQ ID NO: 272 - RNA, 22 nt, Homo sapiens
aaaaguaauugcgguuuuugcc

SEQ ID NO: 273 - RNA, 22 nt, Homo sapiens
uucuccaaaagggagcacuuuc

SEQ ID NO: 274 - RNA, 21 nt, Homo sapiens
ccaccaccgugucugacacuu

SEQ ID NO: 275 - RNA, 22 nt, Homo sapiens
ucuauacagacccuggcuuuuc

SEQ ID NO: 276 - RNA, 22 nt, Homo sapiens
ccuauucuugguuacuugcacg

SEQ ID NO: 277 - RNA, 23 nt, Homo sapiens
acuuaaacguggauguacuugcu

SEQ ID NO: 278 - RNA, 22 nt, Homo sapiens
uucccuuugucauccuaugccu

SEQ ID NO: 279 - RNA, 23 nt, Homo sapiens
gaagugcuucgauuuuggggugu

SEQ ID NO: 280 - RNA, 22 nt, Homo sapiens
gggguuccuggggaugggauuu

SEQ ID NO: 281 - RNA, 17 nt, Homo sapiens
uaauugcuuccauguuu

SEQ ID NO: 282 - RNA, 24 nt, Homo sapiens
acugggggcuuucgggcucugcgu

SEQ ID NO: 283 - RNA, 22 nt, Homo sapiens
cuagguauggucccagggaucc

SEQ ID NO: 284 - RNA, 19 nt, Homo sapiens
ugggcguaucuguaugcua

SEQ ID NO: 285 - RNA, 22 nt, Homo sapiens
aaaaguaaucgcgguuuuuguc

SEQ ID NO: 286 - RNA, 21 nt, Homo sapiens
uuuugcaccuuuuggagugaa

SEQ ID NO: 287 - RNA, 22 nt, Homo sapiens
ggagaaauuauccuuggugugu

SEQ ID NO: 288 - RNA, 22 nt, Homo sapiens
guagauucuccuucuaugagua

SEQ ID NO: 289 - RNA, 22 nt, Homo sapiens
aacgcacuucccuuuagagugu

SEQ ID NO: 290 - RNA, 22 nt, Homo sapiens
aagugccuccuuuuagaguguu

SEQ ID NO: 291 - RNA, 22 nt, Homo sapiens
aacaauauccuggugcugagug

SEQ ID NO: 292 - RNA, 22 nt, Homo sapiens
aaaaguaauugcggucuuuggu

SEQ ID NO: 293 - RNA, 22 nt, Homo sapiens
aggcauugacuucucacuagcu

SEQ ID NO: 294 - RNA, 23 nt, Homo sapiens
aaacucuacuuguccuucugagu

SEQ ID NO: 295 - RNA, 23 nt, Homo sapiens
uauggcuuuucauuccuauguga

SEQ ID NO: 296 - RNA, 22 nt, Homo sapiens
agaauuguggcuggacaucugu

SEQ ID NO: 297 - RNA, 22 nt, Homo sapiens
aaauuauuguacaucggaugag

SEQ ID NO: 298 - RNA, 22 nt, Homo sapiens
ugauugguacgucuguggguag

SEQ ID NO: 299 - RNA, 22 nt, Homo sapiens
gagugccuucuuuuggagcguu

SEQ ID NO: 300 - RNA, 22 nt, Homo sapiens
aaaaguaauugcggauuuugcc

SEQ ID NO: 301 - RNA, 22 nt, Homo sapiens
cuauacggccuccuagcuuucc

SEQ ID NO: 302 - RNA, 22 nt, Homo sapiens
uagguuauccguguugccuucg

SEQ ID NO: 303 - RNA, 22 nt, Homo sapiens
cuuagcagguuguauuaucauu

SEQ ID NO: 304 - RNA, 22 nt, Homo sapiens
uucacauugugcuacugucugc

SEQ ID NO: 305 - RNA, 21 nt, Homo sapiens
uugcucacuguucuucccuag

SEQ ID NO: 306 - RNA, 22 nt, Homo sapiens
ccuauucuugauuacuuguuuc

SEQ ID NO: 307 - RNA, 23 nt, Homo sapiens
uaagugcuuccauguuuugguga

SEQ ID NO: 308 - RNA, 23 nt, Homo sapiens
uauggcuuuuuauuccuauguga

SEQ ID NO: 309 - RNA, 22 nt, Homo sapiens
agagguugcccuuggugaauuc

SEQ ID NO: 310 - RNA, 22 nt, Homo sapiens
aaaaugguucccuuuagagugu

SEQ ID NO: 311 - RNA, 26 nt, Homo sapiens
aaguaguugguuuguaugagaugguu

SEQ ID NO: 312 - RNA, 21 nt, Homo sapiens
ccacaccguaucugacacuuu

SEQ ID NO: 313 - RNA, 22 nt, Homo sapiens
auaauacaugguuaaccucuuu

SEQ ID NO: 314 - RNA, 22 nt, Homo sapiens
ugccugucuacacuugcugugc

SEQ ID NO: 315 - RNA, 22 nt, Homo sapiens
uagguaguuuccuguuguuggg

SEQ ID NO: 316 - RNA, 22 nt, Homo sapiens
uccuucauuccaccggagucug

SEQ ID NO: 317 - RNA, 22 nt, Homo sapiens
caacuagacugugagcuucuag

SEQ ID NO: 318 - RNA, 22 nt, Homo sapiens
accguggcuuucgauuguuacu

SEQ ID NO: 319 - RNA, 23 nt, Homo sapiens
aaagugcugcgacauuugagcgu

SEQ ID NO: 320 - RNA, 22 nt, Homo sapiens
ggggguccccggugcucggauc

SEQ ID NO: 321 - RNA, 22 nt, Homo sapiens
aagugcuuccuuuuagaggguu

SEQ ID NO: 322 - RNA, 22 nt, Homo sapiens
uagguaguuucauguuguuggg

SEQ ID NO: 323 - RNA, 22 nt, Homo sapiens
caaaguuuaagauccuugaagu

SEQ ID NO: 324 - RNA, 22 nt, Homo sapiens
aaaaguauuugcggguuuuguc

SEQ ID NO: 325 - RNA, 21 nt, Homo sapiens
agugaaugauggguucugacc

SEQ ID NO: 326 - RNA, 22 nt, Homo sapiens
aaaaguaauugcgaguuuuacc

SEQ ID NO: 327 - RNA, 21 nt, Homo sapiens
guguugaaacaaucucuacug

SEQ ID NO: 328 - RNA, 22 nt, Homo sapiens
acugauuucuuuugguguucag

SEQ ID NO: 329 - RNA, 23 nt, Homo sapiens
aagugccgccaucuuuugagugu

SEQ ID NO: 330 - RNA, 18 nt, Homo sapiens
cgggcguggugguggggg

SEQ ID NO: 331 - RNA, 22 nt, Homo sapiens
uauugcacuugucccggccugu

SEQ ID NO: 332 - RNA, 22 nt, Homo sapiens
gagcuuauucauaaaagugcag

SEQ ID NO: 333 - RNA, 21 nt, Homo sapiens
gugggcgggggcaggugugug

SEQ ID NO: 334 - RNA, 22 nt, Homo sapiens
uuaucagaaucuccagggguac

SEQ ID NO: 335 - RNA, 21 nt, Homo sapiens
aucacauugccagggauuacc

SEQ ID NO: 336 - RNA, 22 nt, Homo sapiens
ucggccugaccacccaccccac

SEQ ID NO: 337 - RNA, 22 nt, Homo sapiens
uauugcacucgucccggccucc

SEQ ID NO: 338 - RNA, 22 nt, Homo sapiens
uagcaccauuugaaaucgguua

SEQ ID NO: 339 - RNA, 25 nt, Homo sapiens
gggcgacaaagcaagacucuuucuu

SEQ ID NO: 340 - RNA, 22 nt, Homo sapiens
agaucagaaggugauuguggcu

SEQ ID NO: 341 - RNA, 23 nt, Homo sapiens
ugugcuugcucgucccgcccgca

SEQ ID NO: 342 - RNA, 22 nt, Homo sapiens
ccucugaaauucaguucuucag

SEQ ID NO: 343 - RNA, 22 nt, Homo sapiens
uagcaaaaacugcaguuacuuu

SEQ ID NO: 344 - RNA, 24 nt, Homo sapiens
aaagacauaggauagagucaccuc

SEQ ID NO: 345 - RNA, 22 nt, Homo sapiens
accuggcauacaauguagauuu

SEQ ID NO: 346 - RNA, 21 nt, Homo sapiens
aucauagaggaaaauccacgu

SEQ ID NO: 347 - RNA, 22 nt, Homo sapiens
caaagcgcuccccuuuagaggu

SEQ ID NO: 348 - RNA, 23 nt, Homo sapiens
aggaccugcgggacaagauucuu

SEQ ID NO: 349 - RNA, 21 nt, Homo sapiens
gaaagcgcuucucuuuagagg

SEQ ID NO: 350 - RNA, 22 nt, Homo sapiens
caucuuaccggacagugcugga

SEQ ID NO: 351 - RNA, 22 nt, Homo sapiens
uauagggauuggagccguggcg

SEQ ID NO: 352 - RNA, 18 nt, Homo sapiens
gcaugggugguucagugg

SEQ ID NO: 353 - RNA, 20 nt, Homo sapiens
auuccuagaaauuguucaua

SEQ ID NO: 354 - RNA, 23 nt, Homo sapiens
ggcagguucucacccucucuagg

SEQ ID NO: 355 - RNA, 21 nt, Homo sapiens
ucccacguuguggcccagcag

SEQ ID NO: 356 - RNA, 23 nt, Homo sapiens
ugaguaccgccaugucuguuggg

SEQ ID NO: 357 - RNA, 22 nt, Homo sapiens
cuccuauaugaugccuuucuuc

SEQ ID NO: 358 - RNA, 22 nt, Homo sapiens
ugagguaguaaguuguauuguu

SEQ ID NO: 359 - RNA, 22 nt, Homo sapiens
uaauacugucugguaaaaccgu

SEQ ID NO: 360 - RNA, 20 nt, Homo sapiens
guccgcucggcgguggccca

SEQ ID NO: 361 - RNA, 22 nt, Homo sapiens
cugggagaggguuguuuacucc

SEQ ID NO: 362 - RNA, 22 nt, Homo sapiens
uucccuuugucauccuucgccu

SEQ ID NO: 363 - RNA, 22 nt, Homo sapiens
caaaaguaauuguggauuuugu

SEQ ID NO: 364 - RNA, 22 nt, Homo sapiens
aaaaguacuugcggauuuugcu

SEQ ID NO: 365 - RNA, 20 nt, Homo sapiens
aggaauguuccuucuuugcc

SEQ ID NO: 366 - RNA, 22 nt, Homo sapiens
caagcucgcuucuaugggucug

SEQ ID NO: 367 - RNA, 27 nt, Homo sapiens
accuucuuguauaagcacugugcuaaa

SEQ ID NO: 368 - RNA, 24 nt, Homo sapiens
gcugguuucauauggugguuuaga

SEQ ID NO: 369 - RNA, 22 nt, Homo sapiens
uggacggagaacugauaagggu

SEQ ID NO: 370 - RNA, 19 nt, Homo sapiens
gggcgccugugaucccaac

SEQ ID NO: 371 - RNA, 22 nt, Homo sapiens
acuuuaacauggaagugcuuuc

SEQ ID NO: 372 - RNA, 23 nt, Homo sapiens
aucccuugcaggggcuguugggu

SEQ ID NO: 373 - RNA, 22 nt, Homo sapiens
aacgccauuaucacacuaaaua

SEQ ID NO: 374 - RNA, 20 nt, Homo sapiens
ugagcccuguccucccgcag

SEQ ID NO: 375 - RNA, 21 nt, Homo sapiens
aaacuacugaaaaucaaagau

SEQ ID NO: 376 - RNA, 22 nt, Homo sapiens
ugccuacugagcugauaucagu

SEQ ID NO: 377 - RNA, 21 nt, Homo sapiens
ccuggaaacacugagguugug

SEQ ID NO: 378 - RNA, 21 nt, Homo sapiens
gcaggaacuugugagucuccu

SEQ ID NO: 379 - RNA, 21 nt, Homo sapiens
caagucacuagugguuccguu

SEQ ID NO: 380 - RNA, 22 nt, Homo sapiens
augguacccuggcauacugagu

SEQ ID NO: 381 - RNA, 22 nt, Homo sapiens
accaucgaccguugauuguacc

SEQ ID NO: 382 - RNA, 23 nt, Homo sapiens
ucugcucauaccccaugguuucu

SEQ ID NO: 383 - RNA, 23 nt, Homo sapiens
uaagugcuuccauguuugagugu

SEQ ID NO: 384 - RNA, 22 nt, Homo sapiens
auccgcgcucugacucucugcc

SEQ ID NO: 385 - RNA, 22 nt, Homo sapiens
acucaaaaugggggcgcuuucc

SEQ ID NO: 386 - RNA, 19 nt, Homo sapiens
auggauaaggcuuuggcuu

SEQ ID NO: 387 - RNA, 21 nt, Homo sapiens
caucccuugcaugguggaggg

SEQ ID NO: 388 - RNA, 22 nt, Homo sapiens
cuuaucagauuguauuguaauu

SEQ ID NO: 389 - RNA, 22 nt, Homo sapiens
aacaucacagcaagucugugcu

SEQ ID NO: 390 - RNA, 21 nt, Homo sapiens
acagucugcugagguuggagc

SEQ ID NO: 391 - RNA, 22 nt, Homo sapiens
uucacaaggaggugucauuuau

(19) United States
(12) Patent Application Publication
(10) Pub. No.: US 2013/0331290 A1
(43) Pub. Date: Dec. 12, 2013

(54) COMPLEX MIRNA SETS AS NOVEL BIOMARKERS FOR LUNG DISEASES

(71) Applicants: Andreas Keller, Puttlingen (DE); Markus Beier, Weinheim (DE)

(72) Inventors: Andreas Keller, Puttlingen (DE); Markus Beier, Weinheim (DE)

(73) Assignee: febit Holding GmbH, Heidelberg (DE)

(21) Appl. No.: 13/981,089

(22) PCT Filed: Jan. 26, 2012
(86) PCT No.: PCT/EP2012/051241

§ 371 Date: Aug. 21, 2013

Related U.S. Application Data
(60) Provisional application No. 61/437,293, filed on Jan. 28, 2011.

(51) Int. Cl. C12Q 1/68

(52) U.S. Cl. CPC C12Q 1/6883
USPC 506/9; 536/23.1

(57) ABSTRACT

The present invention relates to single polynucleotides or sets of polynucleotides for detecting single miRNAs or sets of miRNAs for diagnosing and/or prognosing of a lung disease in a biological sample from a patient. Further, the present invention relates to means for diagnosing and/or prognosing of a lung disease comprising said polynucleotides or sets of polynucleotides. Furthermore, the present invention relates to a method for diagnosing and/or prognosing of a lung disease based on the determination of expression profiles of single miRNAs or sets of miRNAs representative for a lung disease. In addition, the present invention relates to a kit for diagnosing and/or prognosing of a lung disease comprising means for determining expression profiles of single miRNAs or sets of miRNAs representative for a lung disease and optionally at least one reference and/or algorithm or mathematical function comprised on at least one data carrier.

### TECHNICAL FIELD OF THE INVENTION

The present invention relates to single polynucleotides or sets of polynucleotides for detecting single miRNAs or sets of miRNAs for diagnosing and/or prognosing of a lung disease in a biological sample from a patient. Further, the present invention relates to means for diagnosing and/or prognosing of a lung disease comprising said polynucleotides or sets of polynucleotides. Furthermore, the present invention relates to a method for diagnosing and/or prognosing of a lung disease based on the determination of expression profiles of single miRNAs or sets of miRNAs representative for a lung disease. In addition, the present invention relates to a kit for diagnosing and/or prognosing of a lung disease comprising means for determining expression profiles of single miRNAs or sets of miRNAs representative for a lung disease and optionally at least one reference and/or algorithm or mathematical function comprised on at least one data carrier.

### BACKGROUND OF THE INVENTION

Today, biomarkers play a key role in early diagnosis, risk stratification, and therapeutic management of various diseases. However, many biomarkers were mainly discovered by candidate approach. By contrast, the recent development of high-throughput molecular technologies that allow with a reasonable effort the analysis of whole transcriptomes, proteomes, and metabolomes of individuals at risk, may lead to the discovery of novel biomarkers in an unbiased approach.

MicroRNAs (miRNAs) are a new class of biomarkers. They represent a group of regulatory elements that enable cells to fine-tune complex gene expression cascades in a wide range of biological processes. Since recently it is known that miRNAs are not only present in tissues but also in human blood both as free circulating nucleic acids and in mononuclear cells. This may be due to the fact that miRNAs expressed in diverse tissues or cells may be able to be released into circulating blood. Although the mechanism why miRNAs are found in human blood is not fully understood yet, this finding renders miRNAs to biological markers for diagnostics for various types of diseases based on blood analysis.

Various mlRNA biomarkers have been proposed to indicate several diseases, e.g. diseases of the heart and cardiovascular system or multiple sclerosis. However, there is still a need for novel miRNAs as biomarkers for the detection and/or prediction of other types of diseases. Particularly, the potential role of miRNAs as biomarkers for the diagnosis and/or prognosis of lung diseases has not been systematically evaluated in detail yet. In addition, many of the miRNA biomarkers presently available for diagnosing and/or prognosing of diseases have shortcomings such as low sensitivity, no sufficient specificity or do not allow timely diagnosis. Accordingly, there is still a need to provide novel and efficient miRNAs or sets of miRNAs as biomarkers, effective methods and kits for the diagnosis and/or prognosis of diseases such as lung diseases.

The term lung disease refers to many disorders affecting the lungs, such as asthma, chronic obstructive pulmonary disease (COPD), lung cancer, sarcoidosis and many other breathing problems.

Lung cancer, for example, is a disease which consists of uncontrolled cell growth in tissues of the lung. This growth may lead to metastasis, which is the invasion of adjacent tissue and infiltration beyond the lungs. The vast majority of primary lung cancers are carcinomas of the lung, derived from epithelial cells. Lung cancer, the most common cause of cancer-related death in men and women, is responsible for more than 1.3 million deaths worldwide. The most common symptoms are shortness of breath, coughing (including coughing up blood), and weight loss. Chronic obstructive pulmonary disease (COPD) refers to chronic bronchitis and emphysema, a pair of commonly co-existing diseases of the lungs in which the airways become narrowed. This leads to a limitation of the flow of air to and from the lungs causing shortness of breath. In England, an estimated 842.100 of 50 million people have a diagnosis of COPD; translating into approximately one person in 59 receiving a diagnosis of COPD at some point in their lives. Several miRNA markers have been proposed to indicate lung cancer or COPD. However, many of these markers have shortcomings such as low sensitivity, no sufficient specificity or do not allow timely diagnosis.

Sarcoidosis is characterized by the formation of tiny lumps of cells in various organs in the body. Particularly, sarcoidosis can occur in the lungs. The lumps are called granulomas because they look like grains of sugar or sand. These granulomas can grow and clump together, making several larger groups of lumps. If many granulomas form in the lung, they can affect how the lung works, which can cause symptoms of lung sarcoidosis. Lung sarcoidosis is not a form of cancer. At present, no miRNA markers are known to indicate lung sarcoidosis.

Accordingly, there is still a need to provide novel and efficient miRNAs (e.g. miRNAs signatures or sets of miRNAs) as biomarkers, effective methods and kits for the diagnosis and/or prognosis of lung diseases. Particularly, the potential role of miRNAs present in blood, e.g. human blood, as biomarkers for the diagnosis and/or prognosis of lung diseases in a patient, e.g. human, has not been systematically evaluated yet.

The inventors of the present invention assessed for the first time the expression of miRNAs on a whole-genome level in subjects suffering from a lung disease. They identified novel miRNAs which are significantly dysregulated in a biological sample, e.g. blood sample, of subjects suffering from a lung disease in comparison to healthy controls. The inventors of the present invention revealed that said single miRNAs can predict or determine a lung disease with high specificity, sensitivity and accuracy. The inventors of the present invention also pursued a multiple biomarker strategy to circumvent the above mentioned limitations by adding accuracy and predictive power. In detail, by using a machine learning approach, they identified unique miRNA signatures that can predict or determine a lung disease with even higher power, indicating that both, single miRNAs and especially complex miRNA signatures or sets derived from a biological sample, e.g. blood sample, can be used as novel biomarkers for the diagnosis and/or prognosis of a lung disease in a patient.

Further, distinguishing between the lung diseases lung cancer, sarcoidosis and/or COPD can be extremely difficult. Symptoms are frequently non-specific and often limited to wheezing, shortness of breath and/or chest pain. Many patients with lung cancer exhibit the same symptoms as patients with sarcoidosis and/or COPD. Thus, there is a need for additional diagnostic assays (differential diagnostic assays) that can assess the condition of a lung disease such as lung cancer, sarcoidosis and/or COPD in general and that can distinguish lung cancer from sarcoidosis and/or COPD, distinguish sarcoidosis from lung cancer and/or COPD and distinguish COPD from sarcoidosis and/or lung cancer in particular.

It is also possible that a patient successively or simultaneously develops lung cancer, sarcoidosis and/or COPD. Thus, there is also a need for diagnostic assays that allow the diagnosis and/or prognosis of sarcoidosis and lung cancer, sarcoidosis and COPD, lung cancer and COPD, and sarcoidosis, lung cancer and COPD at the same time.

The inventors of the present invention revealed miRNAs (miRNAs signatures or sets of mirNAs) as biomarkers that allow the differential diagnosis of sarcoidosis with respect to lung cancer and/or COPD, the differential diagnosis of lung cancer with respect to sarcoidosis and/or COPD and the differential diagnosis of COPD with respect to sarcoidosis and/or lung cancer with high specificity, sensitivity and accuracy. They also revelead miRNAs (miRNAs signatures or sets of mirNAs) that can predict or determine lung diseases such as sarcoidosis and lung cancer, sarcoidosis and COPD, lung cancer and COPD, and sarcoidosis, lung cancer and COPD with high specificity, sensitivity and accuracy.

### SUMMARY OF THE INVENTION

In a first aspect, the invention provides one or more polynucleotide(s) for detecting one or more miRNA(s) for diagnosing and/or prognosing of a lung disease in a biological sample from a patient, wherein the nucleotide sequence of said miRNA or the nucleotide sequences of said miRNAs is (are) selected from the group consisting of SEQ ID NOs: 1 to 391.

In a second aspect, the invention provides a method for diagnosing and/or prognosing of a lung disease comprising the steps of:
(i) determining an expression profile of one or more miRNA(s) in a biological sample from a patient, wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto, and
(ii) comparing said expression profile to a reference, wherein the comparison of said expression profile to said reference allows for the diagnosis and/or prognosis of a lung disease, and/or applying an algorithm or a mathematical function to said expression profile, wherein the application of said algorithm or mathematical function to said expression profile allows for the diagnosis and/or prognosis of a lung disease.

In a third aspect, the invention provides means for diagnosing and/or prognosing of a lung disease comprising one or more polynucleotide(s) according to the first aspect.

In a fourth aspect, the invention provides a kit for diagnosing and/or prognosing of a lung disease comprising
(i) means for determining an expression profile of one or more miRNA(s) in a biological sample from a patient, wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto, and
(ii) optionally at least one reference and/or algorithm or mathematical function comprised on at least one data carrier.
   This summary of the invention does not necessarily describe all features of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in this specification and in the appended claims, the singular forms “a”, “an”, and “the” include plural referents, unless the content clearly dictates otherwise. For example, the term “a test compound” also includes “test compounds”.

The terms “microRNA” or “miRNA” refer to single-stranded RNA molecules of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNAs are first transcribed as primary transcripts or pri-miRNAs with a cap and poly-A tail and processed to short, 70 nucleotide stem-loop structures known as pre-miRNAs in the cell nucleus. This processing is performed in animals by a protein complex known as the Microprocessor complex consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC. This strand is known as the guide strand and is selected by the argonaute protein, the catalytically active RNase in the RISC, on the basis of the stability of the 5′ end. The remaining strand, known as the miRNA*, anti-guide (anti-strand), or passenger strand, is degraded as a RISC substrate. Therefore, the miRNA*s are derived from the same hairpin structure like the “normal” miRNAs. So if the “normal” miRNA is then later called the “mature miRNA” or “guide strand”, the miRNA* is the “anti-guide strand” or “passenger strand”.

The terms “microRNA*” or “miRNA*” refer to single-stranded RNA molecules of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The “miRNA*s”, also known as the “anti-guide strands” or “passenger strands”, are mostly complementary to the “mature miRNAs” or “guide strands”, but have usually single-stranded overhangs on each end. There are usually one or more mispairs and there are sometimes extra or missing bases causing single-stranded “bubbles”. The miRNA*s are likely to act in a regulatory fashion as the miRNAs (see also above). In the context of the present invention, the terms “miRNA” and “miRNA*” are interchangeable used. The present invention encompasses (target) miRNAs suitable for the diagnosis and/or prognosis of a lung disease. Said (target) miRNAs are selected from the group consisting of SEQ ID NO: 1 to SEQ ID NO: 391.

The term “miRBase” refers to a well established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download. The sequences of the miRNAs for diagnosis and/or prognosis of a lung disease in FIG. 2 are based on miRBase version 12.0, 13.0 and 14.0.

As used herein, the term “nucleotides” refers to structural components, or building blocks, of DNA and RNA. Nucleotides consist of a base (one of four chemicals: adenine, thymine, guanine, and cytosine) plus a molecule of sugar and one of phosphoric acid. The term “nucleosides” refers to glycosylamine consisting of a nucleobase (often referred to simply base) bound to a ribose or deoxyribose sugar. Examples of nucleosides include cytidine, uridine, adenosine, guanosine, thymidine and inosine. Nucleosides can be phosphorylated by specific kinases in the cell on the sugar's primary alcohol group (—CH2-OH), producing nucleotides, which are the molecular building blocks of DNA and RNA.

The term “polynucleotide”, as used herein, means a molecule of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally spacer elements and/or elongation elements described below. The depiction of a single strand of a polynucleotide also defines the sequence of the complementary strand. Polynucleotides may be single stranded or double stranded, or may contain portions of both double stranded and single stranded sequences. The term “polynucleotide” means a polymer of deoxyribonucleotide or ribonucleotide bases and includes DNA and RNA molecules, both sense and anti-sense strands. In detail, the polynucleotide may be DNA, both cDNA and genomic DNA, RNA, cRNA or a hybrid, where the polynucleotide sequence may contain combinations of deoxyribonucleotide or ribonucleotide bases, and combinations of bases including uracil, adenine, thymine, cytosine, guanine, inosine, xanthine, hypoxanthine, isocytosine and isoguanine Polynucleotides may be obtained by chemical synthesis methods or by recombinant methods.

In the context of the present invention, a polynucleotide as a single polynucleotide strand provides a probe (e.g. miRNA capture probe) that is capable of binding to, hybridizing with, or detecting a target of complementary sequence, such as a nucleotide sequence of a miRNA or miRNA*, through one or more types of chemical bonds, usually through complementary base pairing, usually through hydrogen bond formation. Polynucleotides in their function as probes may bind target sequences, such as nucleotide sequences of miRNAs or miRNAs*, lacking complete complementarity with the polynucleotide sequences depending upon the stringency of the hybridization condition. There may be any number of base pair mismatches which will interfere with hybridization between the target sequence, such as a nucleotide sequence of a miRNA or miRNA*, and the single stranded polynucleotide described herein. However, if the number of mutations is so great that no hybridization can occur under even the least stringent hybridization conditions, the sequences are no complementary sequences. The present invention encompasses polynucleotides in form of single polynucleotide strands as probes for binding to, hybridizing with or detecting complementary sequences of (target) miRNAs for diagnosing and/or prognosing of a lung disease. Said (target) miRNAs are selected from the group consisting of SEQ ID NO: 1 to SEQ ID NO: 391.

The polynucleotide, e.g. the polynucleotide used as a probe for detecting a miRNA or miRNA*, may be unlabeled, directly labeled, or indirectly labeled, such as with biotin to which a streptavidin complex may later bind. The polynucleotide, e.g. the polynucleotide used as a probe for detecting a miRNA or miRNA*, may also be modified, e.g. may comprise an elongation (EL) element. For use in a RAKE or MPEA assay, a polynucleotide with an elongation element may be used as a probe. The elongation element comprises a nucleotide sequence with 1 to 30 nucleotides chosen on the basis of showing low complementarity to potential target sequences, such as nucleotide sequences of miRNAs or miRNAs*, therefore resulting in no or a low degree of cross-hybridization to a target mixture. In one embodiment of the invention, a homomeric sequence stretch Nₙ with n=1 to 30, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and N=A or C, or T or G is preferred. Particularly preferred is a homomeric sequence stretch Nₙ with n=1 to 12, i.e. 1, 2, 3, 4, 5, 6, 7, 8, 9, 10, 11, or 12, and N=A or C, or T or G. The polynucleotide, e.g. the polynucleotide used as a probe for detecting a miRNA or miRNA*, may be present in form of a tandem, i.e. in form of a polynucleotide hybrid of two different or identical polynucleotides, both in the same orientation, i.e. 5′ to 3′ or 3′ to 5′, or in different orientation, i.e. 5′ to 3′ and 3′ to 5′. Said polynucleotide hybrid/tandem may comprise a spacer element. For use in a tandem hybridization assay, the polynucleotide hybrid/tandem as a probe may comprise a spacer (SP) element. The spacer element represents a nucleotide sequence with n=0 to 12, i.e. 0, 1, 2, 3, 4, 5, 6, 7, 8, 9, 10, 11, or 12, nucleotides chosen on the basis of showing low complementarity to potential target sequences, such as nucleotide sequences of miRNAs or anti-miRNAs, therefore resulting in no or a low degree of cross-hybridization to a target mixture. It is preferred that n is 0, i.e. that there is no spacer between the two miRNA sequence stretches. In another embodiment of the invention, a non-homomeric sequence stretch Nₙ with n=1 to 30, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and N=A or C, or T or G is preferred. Particularly preferred is a non-homomeric sequence stretch that can be used as a priming site for a polymerase for a downstream amplification reaction.

For detection purposes, the miRNA(s) or miRNA*(s) may be employed unlabeled, directly labeled, or indirectly labeled, such as with biotin to which a streptavidin complex may later bind.

The term “label”, as used herein, means a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and other entities which are or can be made detectable. A label may be incorporated into nucleic acids at any position, e.g. at the 3′ or 5′ end or internally. The polynucleotide for detecting a miRNA (polynucleotide probe) and/or the miRNA itself may be labeled.

The term “stringent hybridization conditions”, as used herein, means conditions under which a first nucleotide sequence (e.g. polynucleotide in its function as a probe for detecting a miRNA or miRNA*) will hybridize to a second nucleotide sequence (e.g. target sequence such as nucleotide sequence of a miRNA or miRNA*), such as in a complex mixture of nucleotide sequences. Stringent conditions are sequence-dependent and will be different in different circumstances. Stringent conditions may be selected to be about 5 to 10° C. lower than the thermal melting point (Tm) for the specific sequence at a defined ionic strength, pH. The Tm may be the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01 to 1.0 M sodium ion concentration (or other salts) at pH 7.0 to 8.3 and the temperature is at least about 20° C. for short probes (e.g. about 10-35 nucleotides) and up to 60° C. for long probes (e.g. greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.; or 6×SSPE, 10% formamide, 0.01%, Tween 20, 0.1×TE buffer, 0.5 mg/ml BSA, 0.1 mg/ml herring sperm DNA, incubating at 42° C. with wash in 0.5×SSPE and 6×SSPE at 45° C.

The term “antisense”, as used herein, refers to nucleotide sequences which are complementary to a specific DNA or RNA sequence. The term “antisense strand” is used in reference to a nucleic acid strand that is complementary to the “sense” strand.

The term “sensitivity”, as used herein, means a statistical measure of how well a binary classification test correctly identifies a condition, for example how frequently it correctly classifies a lung disease into the correct type out of two or more possible types (e.g. a lung disease type such as a lung cancer type, a sarcoidosis type or a chronic obstructive pulmonary disease (COPD) type, and a healthy type). The sensitivity for class A is the proportion of cases that are determined to belong to class “A” by the test out of the cases that are in class “A”. A theoretical, optimal prediction can achieve 100% sensitivity (i.e. predict all patients from the sick group as sick).

The term “specificity”, as used herein, means a statistical measure of how well a binary classification test correctly identifies a condition, for example how frequently it correctly classifies a lung disease into the correct type out of two or more possible types (e.g. a lung disease type such as a lung cancer type, a sarcoidosis type or a chronic obstructive pulmonary disease (COPD) type, and a healthy type). The specificity for class A is the proportion of cases that are determined to belong to class “not A” by the test out of the cases that are in class “not A”. A theoretical, optimal prediction can achieve 100% specificity (i.e. not predict anyone from the healthy group as sick).

The term “accuracy”, as used herein, means a statistical measure for the correctness of classification or identification of sample types. The accuracy is the proportion of true results (both true positives and true negatives).

The term “lung disease”, as used herein, encompasses any group of clinical symptoms compatible with a lung disease. The term “lung disease” refers to disorders affecting the lungs, such as asthma, lung sarcoidosis (or simply sarcoidosis), chronic obstructive pulmonary disease (COPD), infections like influenza, pneumonia and tuberculosis, lung cancer, and many other breathing problems.

Sarcoidosis is characterized by the formation of tiny lumps of cells in various organs in the body. Lung sarcoidosis is sarcoidosis that occurs in the lungs. The lumps are called granulomas because they look like grains of sugar or sand. These tiny granulomas can grow and clump together, making several larger groups of lumps. If many granulomas form in the lung, they can affect how the lung works, which can cause symptoms of lung sarcoidosis. Lung sarcoidosis is not a form of cancer. Lung sarcoidosis has an active and a non-active phase. In the active phase of lung sarcoidosis, the granulomas form and grow. In this phase, symptoms can develop, and scar tissue can form in the lungs. In the non-active phase of lung sarcoidosis, the inflammation goes down, and the granulomas stay the same size or shrink. However, the scars may remain and cause symptoms.

Lung cancer is a disease which consists of uncontrolled cell growth in tissues of the lung. This growth may lead to metastasis, which is the invasion of adjacent tissue and infiltration beyond the lungs. The vast majority of primary lung cancers are carcinomas of the lung, derived from epithelial cells. Lung cancer is the most common cause of cancer-related death in men and women. The most common symptoms are shortness of breath, coughing (including coughing up blood), and weight loss. The main types of lung cancer are small cell lung carcinoma and non-small cell lung carcinoma.

Chronic obstructive pulmonary disease (COPD), also known as chronic obstructive lung disease (COLD), chronic obstructive airway disease (COAD), chronic airflow limitation (CAL) and chronic obstructive respiratory disease (CORD), refers to chronic bronchitis and emphysema, a pair of commonly co-existing diseases of the lungs in which the airways become narrowed. This leads to a limitation of the flow of air to and from the lungs causing shortness of breath. In contrast to asthma, this limitation is poorly reversible and usually gets progressively worse over time. COPD is caused by noxious particles or gas, most commonly from tobacco smoking, which triggers an abnormal inflammatory response in the lung.

It should be noted that patients having sarcoidosis of the lungs can have or develop at the same time lung cancer and/or COPD, patients having lung cancer can have or develop at the same time COPD and/or sarcoidosis, or patients having COPD can have or develop at the same time lung cancer and/or sarcoidosis.

The term “patient”, as used in the context of the present invention, may mean a subject (e.g. a human subject), which is suspected to be affected by a lung disease (e.g. sarcoidosis, lung cancer, and/or COPD). The patient may be diagnosed to be affected by a lung disease (e.g. sarcoidosis, lung cancer and/or COPD), i.e. diseased, or may be diagnosed to be not affected by a lung disease (e.g. sarcoidosis, lung cancer, and/or COPD), i.e. healthy. The patient may further be prognosed to develop a lung disease (e.g. sarcoidosis, lung cancer, and/or COPD), as the inventors of the present invention surprisingly found that miRNAs representative for a lung disease are already present in a biological sample (e.g. blood sample) before a lung disease occurs or during the early stage of a lung disease.

The term “patient”, as used in the context of the present invention, may also mean a subject (e.g. a human subject), which is affected by a lung disease (e.g. sarcoidosis, lung cancer, and/or COPD), i.e. diseased. The patient may be retested for a lung disease and may be diagnosed to be still affected by a lung disease (e.g. sarcoidosis, lung cancer, and/or COPD), i.e. diseased, or not affected by a lung disease (e.g. sarcoidosis, lung cancer, and/or COPD) anymore, i.e. healthy, for example after therapeutic intervention (e.g. to evaluate the success of surgery and/or chemotherapy).

It should be noted that a patient that is diagnosed as being healthy, i.e. not suffering from a lung disease or a specific form of a lung disease (e.g. sarcoidosis, lung cancer, and/or COPD), or as staying healthy, i.e. not developing a lung disease or a specific form of a lung disease (e.g. sarcoidosis, lung cancer, and/or COPD), may possibly suffer from another disease not tested/known. For example, a patient tested for the presence of a lung disease may be diagnosed to be not affected by a lung disease, but may be affected by an acute coronary syndrome, multiples scleroses, diabetes, and/or gastric cancer which disease(s) has (have) not been tested. Further, for example, a patient tested for the presence of a specific form of a lung disease (e.g. sarcoidosis, lung cancer, and/or COPD) may suffer from an(other) specific form(s) of (a) lung disease(s) which disease(s) has (have) not been tested (e.g. lung cancer, COPD, and/or sarcoidosis).

The patient may be any mammal, including both a human and another mammal, e.g. an animal such as a rabbit, mouse, rat, or monkey. Human subjects as patients are particularly preferred.

The term “(control) subject”, as used in the context of the present invention, may refer to a subject (e.g. human or animal subject) known to be affected by a lung disease (e.g. sarcoidosis, lung cancer, or COPD) (positive control), i.e. diseased. The term “(control) subject”, as used in the context of the present invention, may also refer to a subject (e.g. human or animal subject) known to be not affected by a lung disease (e.g. sarcoidosis, lung cancer, or COPD) (negative control), i.e. healthy. The term “(control) subject” as used in the context of the present invention, may further refer to a subject (e.g. human or animal subject) having a specific stage of a lung disease (e.g. sarcoidosis, lung cancer, or COPD), for example, a subject having an early stage of a lung disease, a subject having an advanced stage of a lung disease, or a subject with subsiding symptoms of a lung disease. It should be noted that a (control) subject that is known to be healthy, i.e. not suffering from a lung disease (e.g. sarcoidosis, lung cancer, and/or COPD), may possibly suffer from another disease not tested/known.

The (control) subject may be any mammal, including both a human and another mammal, e.g. an animal such as a rabbit, mouse, rat, or monkey. Human (control) subjects are particularly preferred.

The inventors of the present invention analysed the expression level of miRNAs (i) in biological samples, e.g. blood samples, of a cohort of controls (healthy subjects) and in biological samples, e.g. blood samples, of subjects suffering from sarcoidosis, (ii) in biological samples, e.g. blood samples, of subjects suffering from sarcoidosis and in biological samples, e.g. blood samples, of subjects suffering from lung cancer, and (iii) in biological samples, e.g. blood samples, of subjects suffering from sarcoidosis and in biological samples, e.g. blood samples, of subjects suffering from COPD. They succeeded in determining the miRNAs that are differentially regulated in biological samples, e.g. blood samples, (i) from subjects having sarcoidosis compared to a cohort of controls (healthy subjects), (ii) from subjects having sarcoidosis compared to subjects having lung cancer, and (iii) from subjects having sarcoidosis compared to subjects having COPD (see experimental section for experimental details). In total, they found 391 miRNAs which are suitable for the diagnosis and/or prognosis of a lung disease (e.g. sarcoidosis, lung cancer, and/or COPD). Said miRNAs are listed in FIG. 2.

Additionally, the inventors of the present invention performed hypothesis tests (e.g. t-test, limma-test) or other measurements (e.g. AUC, mutual information) on the expression level of the found miRNAs in all controls (healthy subjects) and subjects having a lung disease (e.g. sarcoidosis, lung cancer, or COPD). These tests resulted in a significance value (p-value) for each miRNA. This p-value is a measure for the diagnostic power of each miRNA to discriminate, for example, between two clinical conditions, e.g. absence of sarcoidosis (i.e. healthy) and presence of sarcoidosis (i.e. diseased). Since a manifold of tests are carried out, one for each miRNA, the p-values may be too optimistic and, thus, over-estimate the actual discriminatory power. Hence, the p-values are corrected for multiple testing by the Benjamini Hochberg approach.

An overview of the 231 miRNAs that are found to be significantly dysregulated in blood samples of subjects having sarcoidosis compared to a cohort of controls (healthy subjects) and that performed best according to t-test, limma-test or AUC is provided in FIG. 1 (Experimental details: SEQ ID NO: sequence identification number, miRNA: identifier of the miRNA according to miRBase, median g1: median intensity obtained from microarray analysis for healthy controls, median g2: median intensity obtained from microarray analysis for subjects having sarcoidosis, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment). The group of miRNAs comprises miRNAs according to SEQ ID NO: 1 to SEQ ID NO: 231. The miRNAs comprised in this group are sorted in order of their t-test significance (see ttest_adjp=adjusted p-value calculated according to ttest) and intensity as described in more detail in the experimental section. Thus, the most predictive miRNAs are listed first. It should be noted that the lower the ttest_adjp value of a single miRNA, the higher is the diagnostic power of said miRNA. The median intensity obtained from the microarray represents a measure for the abundance of a miRNA in a sample and represents the expression level of this miRNA. The higher the intensity of a single miRNA, the higher the abundance/expression of said miRNA.

Further, an overview of the 229 miRNAs that are found to be significantly dysregulated in blood samples of subjects having sarcoidosis compared to subjects having lung cancer and that performed best according to t-test, limma-test or AUC is provided in FIG. 13 and an overview of the 226 miRNAs that are found to be significantly dysregulated in blood samples of subjects having sarcoidosis compared to subjects having COPD and that performed best according to t-test, limma-test or AUC is provided in FIG. 14. Again, the miRNAs comprised in the group listed in FIG. 13 and the miRNAs comprised in the group listed in FIG. 14 are sorted in order of their t-test significance (see ttest_adjp=adjusted p-value calculated according to ttest) and intensity.

FIG. 18 shows in circle diagrams all 391 miRNAs which have been found to be suitable for the diagnosis and/or prognosis of a lung disease (e.g. sarcoidosis, lung cancer, and/or COPD).

In detail, circle A consisting of areas 1, 2, 4, and 7 represents the 231 miRNAs found to be differentially regulated in blood samples of subjects having sarcoidosis compared to a cohort of controls (healthy subjects). The inventors of the present invention found that these miRNAs, particularly the 62 miRNAs in area 1, are suitable for the diagnosis and/or prognosis of sarcoidosis in a biological sample from a patient. Further, circle B consisting of areas 3, 2, 4, and 5 represents the 229 miRNAs found to be differentially regulated in blood samples of subjects having sarcoidosis compared to subjects having lung cancer. The inventors of the present invention found that these miRNAs, particularly the 60 miRNAs in area 3, are suitable for the diagnosis and/or prognosis of lung cancer in a biological sample from a patient. Furthermore, circle C consisting of areas 6, 7, 4, and 5 represents the 226 miRNAs found to be differentially regulated in blood samples of subjects having sarcoidosis compared to subjects having COPD. The inventors of the present invention found that these miRNAs, particularly the 62 miRNAs in area 6, are suitable for the diagnosis and/or prognosis of COPD in a biological sample from a patient. In this respect, it should be noted that the detection of at least one miRNA comprised in area 1, at least one miRNA comprised in area 3 and/or at least one miRNA comprised in area 6 may allow the diagnosis and/or prognosis of sarcoidosis, lung cancer, and/or COPD, e.g. the diagnosis and/or prognosis of sarcoidosis, lung cancer, COPD, sarcoidosis and lung cancer, sarcoidosis and COPD, lung cancer and COPD, or sarcoidosis, lung cancer and COPD.

In addition, area 2 shared by circles A and B further represents the 43 miRNAs found to be differentially regulated in blood samples of subjects having sarcoidosis compared to a cohort of controls (healthy subjects) and subjects having sarcoidosis compared to subjects having lung cancer; area 7 shared by circles A and C represents the 38 miRNAs found to be differentially regulated in blood samples of subjects having sarcoidosis compared to a cohort of controls (healthy subjects) and subjects having sarcoidosis compared to subjects having COPD; area 5 shared by circles B and C represents the 38 miRNAs found to be differentially regulated in blood samples of subjects having sarcoidosis compared to subjects having lung cancer and subjects having sarcoidosis compared to subjects having COPD; and area 4 shared by circles A, B and C represents the 88 miRNAs found to be differentially regulated in blood samples of subjects having sarcoidosis compared to a cohort of controls (healthy subjects), subjects having sarcoidosis compared to subjects having lung cancer and subjects having sarcoidosis compared to subjects having COPD. The inventors of the present invention also found that these miRNAs that are shared by circles A, B and/or C further allow the differential diagnosis with respect to sarcoidosis, lung cancer, and/or COPD in a biological sample from a patient. This means that (i) the miRNAs that are shared by circles A and B (i.e. miRNAs comprised in area 2) allow to distinguish/differentiate between the lung diseases sarcoidosis and lung cancer, (ii) the miRNAs that are shared by circles A and C (i.e. miRNAs comprised in area 7) allow to distinguish/differentiate between the lung diseases sarcoidosis and COPD, (iii) that the miRNAs that are shared by circles B and C (i.e. miRNAs comprised in area 5) allow to distinguish/differentiate between the lung diseases lung cancer and COPD, and (iv) that the miRNAs that are shared by circles A, B and C (i.e. miRNAs comprised in area 4) allow to distinguish/differentiate between the lung diseases sarcoidosis, lung cancer and COPD.

More specifically, the miRNAs comprised in area 2, for example, allow to diagnose and/or prognose sarcoidosis in a patient and to differentiate said disease with respect to lung cancer, the miRNAs comprised in area 7, for example, allow to diagnose and/or prognose sarcoidosis in a patient and to differentiate said disease with respect to COPD, the miRNAs comprised in area 5, for example, allow to diagnose and/or prognose lung cancer in a patient and to differentiate said disease with respect to COPD, and the miRNAs comprised in area 4, for example, allow to diagnose and/or prognose sarcoidosis in a patient and to differentiate said disease with respect to COPD and lung cancer.

The miRNAs comprised in areas 1, 3 and 6 are valuable as they allow to decide whether (i) the patient is healthy or suffers from sarcoidosis, (ii) the patient is healthy or suffers from lung cancer, or (iii) the patient is healthy or suffers from COPD, respectively. Thus, said miRNAs allow the differentiation between 2 conditions.

The miRNAs comprised in areas 2, 7, and 5, however, are very valuable as they allow the differentiation between 3 conditions. In detail, (i) the miRNAs comprised in area 2 allow the differentiation between the conditions healthy, suffering from sarcoidosis and suffering from lung cancer, (ii) the miRNAs comprised in area 7 allow the differentiation between the conditions healthy, suffering from sarcoidosis and suffering from COPD, and (iii) the miRNAs comprised in area 5 allow the differentiation between the conditions suffering from sarcoidosis, suffering from COPD and suffering from lung cancer. It should be noted that the result of the differential diagnosis in item (i) above may be that the patient is healthy, suffers from sarcoidosis, suffers from lung cancer, or suffers from a combination thereof; that the result of the differential diagnosis in item (ii) above may be that the patient is healthy, suffers from sarcoidosis, suffers from COPD, or suffers from a combination thereof; or that the result of the differential diagnosis in item (iii) above may be that the patient suffers from sarcoidosis, suffers from lung cancer, suffers from COPD, or suffers from a combination thereof. This is caused by the fact that sometimes no differentiation between the different diseases/conditions (e.g. the conditions COPD and lung cancer) is possible as the patient tested also suffers from said disease(s)/condition(s) (e.g. the conditions COPD and lung cancer).

The miRNAs comprised in area 4 are particularly valuable as they allow the differentiation between 4 conditions. In detail, area 4 allows the differentiation between the conditions healthy, suffering from sarcoidosis, suffering from lung cancer, and suffering from COPD. It should be noted that the result of this differential diagnosis may be that the patient is healthy, suffers from sarcoidosis, suffers from lung cancer, suffers from COPD, suffers from sarcoidosis and lung cancer, suffers from sarcoidosis and COPD, suffers from lung cancer and COPD, or suffers from sarcoidosis, lung cancer, and COPD.

Usually the diagnostic power of a single miRNA biomarker is not sufficient to reach high accuracy, specificity and sensitivity, for example, for the discrimination between two clinical conditions, e.g. healthy, i.e. absence of a lung disease, and diseased, i.e. presence of a lung disease. However, the inventors of the present invention surprisingly found that also a single miRNA, wherein the nucleotide sequence of said miRNA is selected from the group consisting of SEQ ID NO: 1 to SEQ ID NO: 391, can predict or determine with high diagnostic accuracy, specificity and sensitivity a lung disease (e.g. sarcoidosis, lung cancer, and/or COPD) in a patient, e.g. a human.

The inventors of the present invention also employed more than one miRNA biomarker, i.e. sets (signatures) of miRNA biomarkers, to further increase and/or improve the performance for diagnosing and/or prognosing of patients, e.g. humans, having a lung disease (e.g. sarcoidosis, lung cancer, and/or COPD).

In order to be able to better discriminate, for example, between two or more clinical conditions, e.g. the presence of a lung disease such as sarcoidosis, lung cancer, and/or COPD and the absence of a lung disease such as sarcoidosis, lung cancer, and/or COPD, for a defined set (signature) of miRNA biomarkers, the inventors of the present invention applied a machine learning approach (e.g. t-test, AUC, support vector machine, hierarchical clustering, or k-means) which leads to an algorithm or a mathematical function that is trained by reference data (i.e. data of reference miRNA expression profiles from the two or more clinical conditions, e.g. the presence of a lung disease such as sarcoidosis, lung cancer, and/or COPD and the absence of a lung disease such as sarcoidosis, lung cancer, and/or COPD, for the defined set (signature) of miRNA markers) to discriminate between the two or more statistical classes (i.e. two or more clinical conditions), e.g. the presence of a lung disease such as sarcoidosis, lung cancer, and/or COPD and the absence of a lung disease such as sarcoidosis, lung cancer, and/or COPD.

The inventors of the present invention surprisingly found that this approach yields miRNA sets (signatures) that can predict or determine with very high diagnostic accuracy, specificity and sensitivity a lung disease, e.g. sarcoidosis, lung cancer, and/or COPD, in a patient, e.g. a human or an animal. Said miRNA sets (signatures) comprise at least two miRNAs, wherein the nucleotide sequences of said miRNAs are selected from the group consisting of SEQ ID NO: 1 to SEQ ID NO: 391 (see for example FIGS. 4 to 12 and FIGS. 15 to 17).

An exemplarily approach to arrive at miRNA sets/signatures that correlate with the lung disease sarcoidosis is summarized below:
Step 1: Total RNA (or subfractions thereof) is extracted from a blood (including plasma, serum, PBMC or other blood fractions) sample of a subject(s) with sarcoidosis using suitable kits and/or purification methods.
Step 2: From the respective sample, the quantity (expression level) of one miRNA or sets of at least two miRNAs selected from the group consisting of SEQ ID NO: 1 to SEQ ID NO: 231 is measured using experimental techniques. These techniques include, but are not restricted to, array based approaches, amplification methods (PCR, RT-PCR, or qPCR), sequencing, next generation sequencing, and/or mass spectroscopy.
Step 3: In order to gather information on the diagnostic/prognostic value and the redundancy of each of the single miRNA biomarkers, mathematical methods are applied. These methods include, but are not restricted to, basic mathematic approaches (e.g. Fold Quotients, Signal-to-Noise ratios, Correlation), statistical methods as hypothesis tests (e.g. t-test, Wilcoxon-Mann-Whitney test), the Area under the Receiver operator Characteristics Curve, information theory approaches, (e.g. the Mutual Information, Cross-entropy), probability theory (e.g. joint and conditional probabilities) or combinations and modifications of the previously mentioned methods.
Step 4: The information gathered in step 3) is used to estimate for each miRNA biomarker the diagnostic content or value. Usually, however, this diagnostic value is too small to get a highly accurate diagnosis with accuracy rates, specificities and sensitivities beyond the 80% barrier. The diagnostic content of the miRNAs suitable for diagnosing/prognosing sarcoidosis is listed in FIG. 1 (Experimental details: SEQ ID NO: sequence identification number, miRNA: identifier of the miRNA according to miRBase, median g1: median intensity obtained from microarray analysis for healthy controls, median g2: median intensity obtained from microarray analysis for individuals with sarcoidosis, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment). This Figure includes the miRNAs according to SEQ ID NO: 1 to SEQ ID NO: 231.
Step 5: In order to increase the performance for diagnosing/prognosing of individuals suffering from sarcoidosis, more than one miRNA biomarker needs to be employed. Thus statistical learning/machine learning/bioinformatics/computational approaches are applied for set selection in order to select/define sets of miRNA biomarkers (comprising miRNAs SEQ ID NO: 1 to SEQ ID NO: 231) that are tailored for the detection of sarcoidosis. These techniques include, but are not restricted to, Wrapper subset selection techniques (e.g. forward step-wise, backward step-wise, combinatorial approaches, optimization approaches), filter subset selection methods (e.g. the methods mentioned in Step 3), principal component analysis, or combinations and modifications of such methods (e.g. hybrid approaches).
Step 6: The subsets, selected/defined in Step 5, which may range from only a small number (at least two for the set) to all measured biomarkers, are then used to carry out a diagnosis/prognosis of sarcoidosis. To this end, statistical learning/machine learning/bioinformatics/computational approaches are applied that include but are not restricted to any type of supervised or unsupervised analysis: classification techniques (e.g. naïve Bayes, Linear Discriminant Analysis, Quadratic Discriminant Analysis Neural Nets, Tree based approaches, Support Vector Machines, Nearest Neighbour Approaches), Regression techniques (e.g. linear Regression, Multiple Regression, logistic regression, probit regression, ordinal logistic regression ordinal Probit-Regression, Poisson Regression, negative binomial Regression, multinomial logistic Regression, truncated regression), Clustering techniques (e.g. k-means clustering, hierarchical clustering, PCA), Adaptations, extensions, and combinations of the previously mentioned approaches.
Step 7: By combination of subset selection (Step 5) and machine learning approaches (Step 6) an algorithm or a mathematical function for diagnosing/prognosing sarcoidosis is obtained. This algorithm or mathematical function is applied to a miRNA expression profile (miRNA expression profile data) of an individual (patient) to be diagnosed for sarcoidosis.

Thus, in a first aspect, the present invention relates to (the use of) one or more polynucleotide(s) for detecting one or more miRNA(s) for diagnosing and/or prognosing of a lung disease in a biological sample from a patient, wherein the nucleotide sequence of said miRNA or the nucleotide sequences of said miRNAs is (are) selected from the group consisting of SEQ ID NOs: 1 to 391.

The terms “one polynucleotide” and “a single polynucleotide” or “one miRNA” and “a single miRNA” can interchangeable be used in the context of the present invention. Further, “more than one polynucleotides” (i.e. at least two polynucleotides) or “more than one miRNAs” (i.e. at least two miRNAs) may be comprised in a set (e.g. a set of polynucleotides such as a set comprising at least two polynucleotides or a set of miRNAs such as a set comprising at least two miRNAs). The miRNAs which are comprised in a set may represent miRNA signatures.

Preferably the lung disease is sarcoidosis, lung cancer, and/or chronic obstructive pulmonary disease (COPD), e.g. (i), sarcoidosis, (ii) lung cancer, (iii) COPD, (iv) sarcoidosis and lung cancer, (v) sarcoidosis and COPD, (vi) lung cancer and COPD, or (vii) sarcoidosis, lung cancer, and COPD.

The term “biological sample”, as used in the context of the present invention, refers to any biological sample containing miRNA(s). Said biological sample may be a body fluid sample, a tissue sample, or a cell sample. For example, biological samples encompassed by the present invention are tissue (e.g. section or explant) samples, single cell samples, cell colony samples, cell culture samples, blood (e.g. whole blood or a blood fraction such as serum or plasma) samples, urine samples, or samples from other peripheral sources. Said biological samples may be mixed or pooled, e.g. a biological sample may be a mixture of blood and urine samples. A “biological sample” may be provided by removing cell(s), cell colonies, an explant, or a section from a patient or control subject, but may also be provided by using a previously isolated sample. For example, a tissue sample may be removed from a patient or control subject by conventional biopsy techniques or a blood sample may be taken from a patient or control subject by conventional blood collection techniques. The biological sample, e.g. tissue or blood sample, may be obtained from a patient prior to initiation of the therapeutic treatment, during the therapeutic treatment, and/or after the therapeutic treatment. In one embodiment, the therapeutical treatment is monitored on the basis of the detection of the miRNA(s) by the polynucleotide(s) of the invention. It is also preferred that total RNA or a subfraction thereof, isolated (e.g. extracted) from a biological sample of a patient (e.g. human or animal), is used for detecting the miRNA(s) by the polynucleotide(s) of the invention.

It is preferred that the biological sample is a body fluid sample, a tissue sample, a cell colony sample, a single cell sample or a cell culture sample. Said biological samples may be mixed or pooled, e.g. the biological sample may be a mixture of a body fluid sample and a tissue sample or a mixture of a body fluid sample and a cell culture sample.

More preferably, the tissue sample from a patient (e.g. human or animal) is a section or an explant sample.

It is preferred that the tissue sample from a patient (e.g. human or animal) has a weight of between 0.1 and 500 mg, more preferably of between 0.5 and 250 mg, and most preferably of between 1 and 50 mg, i.e. 0.1, 0.2, 0.3, 0.4, 0.5, 0.6, 0.7, 0.8, 0.9, 1, 2, 2.5, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg.

It is also preferred that the cell sample (e.g. cell colony sample or cell culture sample) from a patient (e.g. human or animal) consists of between 10² and 10¹⁰ cells, more preferably of between 10³ and 10⁷ cells, and most preferably of between 10⁴ and 10⁶ cells, i.e. 10², 10³, 10⁴, 10⁵, 10⁶, 10⁷, 10⁸, 10⁹, or 10¹⁰ cells.

The term “body fluid sample”, as used in the context of the present invention, refers to a liquid sample derived from the body of a subject, e.g. human or animal. Said body fluid sample may be a blood, urine, sputum, breast milk, cerebrospinal fluid, cerumen (earwax), endolymph, perilymph, gastric juice, mucus, peritoneal fluid, pleural fluid, saliva, sebum (skin oil), semen, sweat, tears, vaginal secretion, or vomit sample including components or fractions thereof. Said body fluid samples may be mixed or pooled. Thus, a body fluid sample may be a mixture of blood and urine samples or a mixture of blood and cerebrospinal fluid samples. A “body fluid sample” may be provided by removing a body liquid from a subject, but may also be provided by using previously isolated body fluid sample material. In the context of the present invention said “body fluid sample” allows for a non-invasive diagnosis/and or prognosis of a subject, e.g. human or animal.

It is preferred that the body fluid sample from a subject (e.g. human or animal) has a volume of between 0.1 and 20 ml, more preferably of between 0.5 and 10 ml, more preferably between 1 and 8 ml and most preferably between 2 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

More preferably, the body fluid sample is a blood sample or an urine sample or mixtures thereof.

Most preferably, the blood sample from a patient (e.g. human or animal) is whole blood or a blood fraction such as serum or plasma. It is also particularly preferred to use blood cells also known as hemopoietic cells. The term “hemopoietic cells” refers to mature cell types and their immature precursors that are identifiable either by morphology or, mostly, by a distinct pattern of cell surface markers. Said term is used to distinguish these cells from other cell types found in the body and also includes T-cells and distinctive subsets, which are the only hematopoietic cells that are not generated in the bone marrow. Said blood cells may be erythrocytes, leukocytes and/or thrombocytes. Peripheral blood mononuclear cells (PBMCs) such as lymphocytes, monocytes or macrophages may also be used.

It is preferred that the blood sample from a patient (e.g. human or animal) has a volume of between 0.1 and 20 ml, more preferably of between 0.5 and 10 ml, and most preferably of between 1 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

It is further preferred that the blood sample is collected by a blood collection tube. Preferably, the blood collection tube includes means for stabilizing the RNA-fraction, especially the small RNA fraction within the blood sample. Not limiting examples for blood collection tubes already including a RNA-stabilization agent are PAXgene tubes (www.Preanalytix.com), or Tempus Blood RNA Tubes (Ambion, Applied Biosystems). Conventionally blood collection tubes, to which optionally a RNA-stabilizing agent like RNAlater (Ambion) can be added, are EDTA-, Heparin-, or Serum-tubes.

It is also preferred that the urine sample from a patient (e.g. human or animal) has a volume of between 0.1 and 20 ml, more preferably of between 0.5 and 15 ml, and most preferably of between 3 and 12 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

It is preferred that the polynucleotides of the invention, preferably comprised in a set, are for detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 miRNAs, preferably comprised in a set, for diagnosing and/or prognosing of a lung disease, preferably sarcoidosis, lung cancer, and/or chronic obstructive pulmonary disease (COPD), in a biological sample from a patient, wherein the nucleotide sequence of said miRNA or the nucleotide sequences of said miRNAs is (are) selected from the group consisting of SEQ ID NOs: 1 to 391.

Preferably, the polynucleotide(s) is (are) for detecting one or more miRNA(s) for diagnosing and/or prognosing of sarcoidosis, wherein the nucleotide sequence of said miRNA or the nucleotide sequences of said miRNAs is (are) selected from the group consisting of SEQ ID NOs: 1 to 231 (see lists 1, 2, 4 and 7 of FIG. 18). In this respect, it is particularly preferred that the polynucleotide(s) is (are) for detecting one or more miRNA(s) for diagnosing and/or prognosing of sarcoidosis, wherein the nucleotide sequence of said miRNA or the nucleotide sequences of said miRNAs is (are) selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (see list 1 of FIG. 18). It is further particularly preferred that the polynucleotides are for detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 miRNAs for diagnosing and/or prognosing of sarcoidosis, wherein the nucleotide sequences of said miRNAs are selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (see list 1 of FIG. 18).

More preferably, said polynucleotide(s) is (are) further for detecting one or more miRNA(s) for differential diagnosis (of sarcoidosis) with respect to lung cancer and/or COPD, wherein the nucleotide sequence of said miRNA or the nucleotide sequences of said miRNAs is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18).

Most preferably,
(i) said polynucleotide(s) is (are) for detecting one or more miRNA(s) for differential diagnosis (of sarcoidosis) with respect to lung cancer, wherein the nucleotide sequence of said miRNA or the nucleotide sequences of said miRNAs is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (see list 2 of FIG. 18),
(ii) said polynucleotide(s) is (are) for detecting one or more miRNA(s) for differential diagnosis (of sarcoidosis) with respect to lung cancer and COPD, wherein the nucleotide sequence of said miRNA or the nucleotide sequences of said miRNAs is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18), or
(iii) said polynucleotide(s) is (are) for detecting one or more miRNA(s) for differential diagnosis (of sarcoidosis) with respect to COPD, wherein the nucleotide sequence of said miRNA or the nucleotide sequences of said miRNAs is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18).

In preferred embodiments, the nucleotide sequences of said miRNAs are the nucleotide sequences according to Set No. 1 to 42 (i.e. SH-1 to SH-42) of FIG. 15.

Preferably, the polynucleotide(s) is (are) for detecting one or more miRNA(s) for diagnosing and/or prognosing of lung cancer, wherein the nucleotide sequence of said miRNA or the nucleotide sequences of said miRNAs is (are) selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, 329 (see list 3 of FIG. 18), 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). In this respect, it is particularly preferred that the polynucleotide(s) is (are) for detecting one or more miRNA(s) for diagnosing and/or prognosing of lung cancer, wherein the nucleotide sequence of said miRNA or the nucleotide sequences of said miRNAs is (are) selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, and 329 (list 3 of FIG. 18). It is further particularly preferred that the polynucleotides are for detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 miRNAs for diagnosing and/or prognosing of lung cancer, wherein the nucleotide sequences of said miRNAs are selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, and 329 (list 3 of FIG. 18).

More preferably, said polynucleotide(s) is (are) further for detecting one or more miRNA(s) for differential diagnosis (of lung cancer) with respect to sarcoidosis and/or COPD, wherein the nucleotide sequence of said miRNA or the nucleotide sequences of said miRNAs is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18).

Most preferably,
(i) said polynucleotide(s) is (are) for detecting one or more miRNA(s) for differential diagnosis (of lung cancer) with respect to sarcoidosis, wherein the nucleotide sequence of said miRNA or the nucleotide sequences of said miRNAs is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (see list 2 of FIG. 18),
(ii) said polynucleotide(s) is (are) for detecting one or more miRNA(s) for differential diagnosis (of lung cancer) with respect to sarcoidosis and COPD, and wherein the nucleotide sequence of said miRNA or the nucleotide sequences of said miRNAs is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18), or
(iii) said polynucleotide(s) is (are) for detecting one or more miRNA(s) for differential diagnosis (of lung cancer) with respect to COPD, and wherein the nucleotide sequence of said miRNA or the nucleotide sequences of said miRNAs is (are) selected from the group consisting of SEQ ID NOs: 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18).

In preferred embodiments, the nucleotide sequences of said miRNAs are the nucleotide sequences according to Set No. 1 to 42 (i.e. SL-1 to SL-42) of FIG. 16.

Preferably, the polynucleotide(s) is (are) for detecting one or more miRNA(s) for diagnosing and/or prognosing of COPD, wherein the nucleotide sequence of said miRNA or the nucleotide sequences of said miRNAs is (are) selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391 (see list 6 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, 231 (see list 7 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). In this respect, it is particularly preferred that the polynucleotide(s) is (are) for detecting one or more miRNA(s) for diagnosing and/or prognosing of COPD, wherein the nucleotide sequence of said miRNA or the nucleotide sequences of said miRNAs is (are) selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, and 391 (see list 6 of FIG. 18). It is further particularly preferred that the polynucleotides are for detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 miRNAs for diagnosing and/or prognosing of COPD, wherein the nucleotide sequences of said miRNAs are selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, and 391 (see list 6 of FIG. 18).

More preferably, said polynucleotide(s) is (are) further for detecting one or more miRNA(s) for differential diagnosis (of COPD) with respect to sarcoidosis and/or lung cancer, and wherein the nucleotide sequence of said miRNA or the nucleotide sequences of said miRNAs is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, 231 (see list 7 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18).

Most preferably,
(i) said polynucleotide(s) is (are) for detecting one or more miRNA(s) for differential diagnosis (of COPD) with respect to sarcoidosis, wherein the nucleotide sequence of said miRNA or the nucleotide sequences of said miRNAs is (are) selected from SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18),
(ii) said polynucleotide(s) is (are) for detecting one or more miRNA(s) for differential diagnosis (of COPD) with respect to sarcoidosis and lung cancer, wherein the nucleotide sequence of said miRNA or the nucleotide sequences of said miRNAs is (are) selected from SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18), or
(iii) said polynucleotide(s) is (are) for detecting one or more miRNA(s) for differential diagnosis (of COPD) with respect to lung cancer, wherein the nucleotide sequence of said miRNA or the nucleotide sequences of said miRNAs is (are) selected from SEQ ID NOs: 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18).

In preferred embodiments, the nucleotide sequences of said miRNAs are the nucleotide sequences according to Set No. 1 to 42 (i.e. SC-1 to SC-42) of FIG. 17.

With respect to the above mentioned embodiments of the first aspect of the present invention, it is preferred that the nucleotide sequences of the miRNAs have (i) SEQ ID NO: 2 and SEQ ID NO: 3, (ii) SEQ ID NO: 3 and SEQ ID NO: 4, (iii) SEQ ID NO: 5, SEQ ID NO: 6, and SEQ ID NO: 7, (iv) SEQ ID NO: 8, SEQ ID NO: 9, and SEQ ID NO: 10, (v) SEQ ID NO: 11, SEQ ID NO: 12, and SEQ ID NO: 13, (vi) SEQ ID NO: 14, SEQ ID NO: 15, and SEQ ID NO: 16, (vii) SEQ ID NO: 17, SEQ ID NO: 18, SEQ ID NO: 19, and SEQ ID NO: 20, (viii) SEQ ID NO: 21, SEQ ID NO: 22, SEQ ID NO: 23, and SEQ ID NO: 24, (ix) SEQ ID NO: 25, SEQ ID NO: 26, SEQ ID NO: 27, SEQ ID NO: 28, SEQ ID NO: 29, and SEQ ID NO: 30, (x) SEQ ID NO: 31, SEQ ID NO: 32, SEQ ID NO: 33, SEQ ID NO: 34, SEQ ID NO: 35, and SEQ ID NO: 36, or (xi) SEQ ID NO: 37, SEQ ID NO: 38, SEQ ID NO: 39, SEQ ID NO: 40. Said miRNAs (miRNA combinations) may also be further combined with each other, e.g. (i) with (ii), (ii) with (iii), (iii) with (iv), (iv) with (v), (v) with (vi), (vi) with (vii), or any other possible combination.

It is also preferred that the nucleotide sequences of the miRNAs have (i) SEQ ID NO: 1 and SEQ ID NO: 2, (ii) SEQ ID NO: 1 and SEQ ID NO: 3, (iii) SEQ ID NO: 1 and SEQ ID NO: 4, (iv) SEQ ID NO: 4 and SEQ ID NO: 5, (v) SEQ ID NO: 5 and SEQ ID NO: 6, (vi) SEQ ID NO: 6 and SEQ ID NO: 7, (vii) SEQ ID NO: 7 and SEQ ID NO: 8, (viii) SEQ ID NO: 8 and SEQ ID NO: 9, (ix) SEQ ID NO: 9 and SEQ ID NO. 10, (x) SEQ ID NO: 10 and SEQ ID NO: 11, or (xi) SEQ ID NO: 11 and SEQ ID NO: 12. Said miRNAs (miRNA combinations) may also be further combined with each other, e.g. (i) with (ii), (ii) with (iii), (iii) with (iv), (iv) with (v), (v) with (vi), (vi) with (vii), or any other possible combination. It is also preferred that the nucleotide sequences of the miRNAs have (i) SEQ ID NO: 1 to SEQ ID NO: 3, (ii) SEQ ID NO: 2 to SEQ ID NO: 4, (iii) SEQ ID NO: 3 to SEQ ID NO: 5, (iv) SEQ ID NO: 4 to SEQ ID NO: 6, (v) SEQ ID NO: 5 to SEQ ID NO: 7, (vi) SEQ ID NO: 6 to SEQ ID NO: 8, (vii) SEQ ID NO: 7 to SEQ ID NO: 9, (viii) SEQ ID NO: 8 to SEQ ID NO: 10, (ix) SEQ ID NO: 9 to SEQ ID NO: 11, (x) SEQ ID NO: 10 to SEQ ID NO: 12, or (xi) SEQ ID NO: 11 to SEQ ID NO: 13. Said miRNAs (miRNA combinations) may also be further combined with each other, e.g. (i) with (ii), (ii) with (iii), (iii) with (iv), (iv) with (v), (v) with (vi), (vi) with (vii), or any other possible combination.

It is particularly preferred that the nucleotide sequences of the miRNAs have (i) SEQ ID NO: 1, SEQ ID NO: 2, SEQ ID NO: 3, SEQ ID NO: 4, SEQ ID NO: 68, SEQ ID NO: 5, SEQ ID NO: 6, SEQ ID NO: 7, SEQ ID NO: 69, and SEQ ID NO: 8, (ii) SEQ ID NO: 68, SEQ ID NO: 5, SEQ ID NO: 6, SEQ ID NO: 7, SEQ ID NO: 69, SEQ ID NO: 8, SEQ ID NO: 70, SEQ ID NO: 105, SEQ ID NO: 9, and SEQ ID NO: 10, (iii) SEQ ID NO: 8, SEQ ID NO: 70, SEQ ID NO: 105, SEQ ID NO: 9, SEQ ID NO: 10, SEQ ID NO: 11, SEQ ID NO: 12, SEQ ID NO: 13, SEQ ID NO: 14, and SEQ ID NO: 114, (iv) SEQ ID NO: 68, SEQ ID NO: 1, SEQ ID NO: 4, SEQ ID NO: 69, SEQ ID NO: 3, SEQ ID NO: 5, SEQ ID NO: 8, SEQ ID NO: 70, SEQ ID NO: 7, and SEQ ID NO: 6, (v) SEQ ID NO: 3, SEQ ID NO: 5, SEQ ID NO: 8, SEQ ID NO: 70, SEQ ID NO: 7, SEQ ID NO: 6, SEQ ID NO: 12, SEQ ID NO: 9, SEQ ID NO: 105, and SEQ ID NO: 107, or (vi) SEQ ID NO: 6, SEQ ID NO: 12, SEQ ID NO: 9, SEQ ID NO: 105, SEQ ID NO: 107, SEQ ID NO: 71, SEQ ID NO: 17, SEQ ID NO: 159, SEQ ID NO: 19, and SEQ ID NO: 106. Said miRNAs (miRNA combinations) may also be further combined with each other, e.g. (i) with (ii), (ii) with (iii), (iii) with (iv), (iv) with (v), (v) with (vi), (vi) with (vii), or any other possible combination.

In another preferred embodiment, the nucleotide sequence of the (single) miRNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or is selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20. It is particularly preferred that the nucleotide sequence of the (single) miRNA is selected from the group consisting of SEQ ID NO: 1, SEQ ID NO: 2, SEQ ID NO: 3, SEQ ID NO: 4, and SEQ ID NO: 5.

It should be noted that the above mentioned polynucleotides (at least two polynucleotides) according to the present invention may be comprised in a set in order to detect miRNAs (at least two miRNAs) which may be also comprised in a set for diagnosing and/or prognosing of a lung disease such as sarcoidosis, lung cancer, and/or COPD, wherein the nucleotide sequences of said miRNAs are selected from the group consisting of SEQ ID NOs: 1 to 391. For example, said set of polynucleotides may comprise, essentially consist of, or consist of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 polynucleotides for detecting a set of miRNAs which may comprise, essentially consist, or consist of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 miRNAs for diagnosing and/or prognosing of a lung disease such as sarcoidosis, lung cancer, and/or COPD in a biological sample from a patient, wherein the nucleotide sequences of said miRNAs are selected from the group consisting of SEQ ID NOs: 1 to 391.

It is preferred that
(i) the polynucleotide of the present invention is complementary to the miRNA, wherein the nucleotide sequence of said miRNA is selected from the group consisting of SEQ ID NOs: 1 to 391, or the polynucleotides of the present invention are complementary to the miRNAs, wherein the nucleotide sequences of said miRNAs are selected from the group consisting of SEQ ID NOs: 1 to 391,
(ii) the polynucleotide is a fragment of the polynucleotide according to (i), preferably the polynucleotide is a fragment which is between 1 and 12, more preferably between 1 and 8, and most preferably between 1 and 5 or 1 and 3, i.e. 1, 2, 3, 4, 5, 6, 7, 8, 9, 10, 11, or 12, nucleotides shorter than the polynucleotide according to (i), or the polynucleotides are fragments of the polynucleotides according to (i), preferably the polynucleotides are fragments which are between 1 and 12, more preferably between 1 and 8, and most preferably between 1 and 5 or 1 and 3, i.e. 1, 2, 3, 4, 5, 6, 7, 8, 9, 10, 11, or 12, nucleotides shorter than the polynucleotides according to (i), or
(iii) the polynucleotide has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the polynucleotide sequence of the polynucleotide according to (i) or polynucleotide fragment according to (ii), or the polynucleotides have at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the polynucleotide sequences of the polynucleotides according to (i) or polynucleotide fragments according to (ii).

It is particularly preferred that the polynucleotide as defined in (iii) has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity over a continuous stretch of at least 10, 11, 12, 13, 14, 15, 16, 17, 18, 19, 20 or more nucleotides, preferably over the whole length, to the polynucleotide sequence of the polynucleotide according to (i) or polynucleotide fragment according to (ii), or that the polynucleotides as defined in (iii) have at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity over a continuous stretch of at least 10, 11, 12, 13, 14, 15, 16, 17, 18, 19, 20 or more nucleotides, preferably over the whole length, to the polynucleotide sequences of the polynucleotides according to (i) or polynucleotide fragments according to (ii).

In addition, the polynucleotide or polynucleotides as defined in (ii) (i.e. polynucleotide fragment(s)) or (iii) (i.e. polynucleotide variant(s) or polynucleotide fragment variant(s)) is (are) only regarded as a polynucleotide or polynucleotides as defined in (ii) (i.e. polynucleotide fragment(s)) or (iii) (i.e. polynucleotide variant(s) or polynucleotide fragment variant(s)) within the context of the present invention, if it is or they are still capable of binding to, hybridizing with, or detecting a target miRNA of complementary sequence or target miRNAs of complementary sequences, e.g. the respective target miRNA(s) according to SEQ ID NO: 1 to SEQ ID NO: 391, through one or more types of chemical bonds, usually through complementary base pairing, usually through hydrogen bond formation under stringent hybridization conditions. The skilled person can readily assess whether a polynucleotide or polynucleotides as defined in (ii) (i.e. polynucleotide fragment(s)) or (iii) (i.e. polynucleotide variant(s) or polynucleotide fragment variant(s)) is (are) still capable of binding to, hybridizing with, recognizing or detecting a target miRNA of complementary sequence or target miRNAs of complementary sequences, e.g. the respective target miRNA(s) according to SEQ ID NO: 1 to SEQ ID NO: 391. Suitable assays to determine whether hybridization under stringent conditions still occurs are well known in the art. However, as an example, a suitable assay to determine whether hybridization still occurs comprises the steps of: (a) incubating the polynucleotide or polynucleotides as defined in (ii) or (iii) attached onto a biochip with the miRNA(s) of complementary sequence(s), e.g. the respective target miRNA(s) according to SEQ ID NO: 1 to SEQ ID NO: 391, labeled with biotin under stringent hybridization conditions, (b) washing the biochip to remove unspecific bindings, (c) subjecting the biochip to a detection system, and (c) analyzing whether the polynucleotide(s) can still hybridize with the target miRNA(s) of complementary sequence(s), e.g. the respective target miRNA(s) according to SEQ ID NO: 1 to SEQ ID NO: 391. As a positive control, the respective non-mutated and not fragmented polynucleotide as defined in (i) may be used. Preferably stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.; or 6×SSPE, 10% formamide, 0.01%, Tween 20, 0.1×TE buffer, 0.5 mg/ml BSA, 0.1 mg/ml herring sperm DNA, incubating at 42° C. with wash in 0.5×SSPE and 6×SSPE at 45° C.

In a second aspect, the present invention provides a method for diagnosing and/or prognosing of a lung disease comprising the steps of:
(i) determining an expression profile of one or more miRNA(s) in a biological sample from a patient, wherein the nucleotide sequence of said miRNA or the nucleotide sequences of said miRNAs is (are) selected from the group consisting of SEQ ID NOs: 1 to 391, a fragment thereof, and a sequence having at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity thereto, and
(ii) comparing said expression profile (data) to a reference, wherein the comparison of said expression profile (data) to said reference allows for the diagnosis and/or prognosis of a lung disease, and/or applying an algorithm or a mathematical function to said expression profile, wherein the application of said algorithm or mathematical function to said expression profile allows for the diagnosis and/or prognosis of a lung disease.

Preferably the lung disease is sarcoidosis, lung cancer, and/or chronic obstructive pulmonary disease (COPD), e.g. (i), sarcoidosis, (ii) lung cancer, (iii) COPD, (iv) sarcoidosis and lung cancer, (v) sarcoidosis and COPD, (vi) lung cancer and COPD, or (vii) sarcoidosis, lung cancer, and COPD.

It is preferred that the patient is a mammal including both a human and another mammal, e.g. an animal such as a mouse, a rat, a rabbit, or a monkey. It is particularly preferred that the patient is a human.

The term “miRNA expression profile”, as used herein, represents the expression level of a single (1) miRNA or a collection of expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, or 390 miRNAs, or of 391 miRNAs, preferably comprised in a set, wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

It is preferred that the biological sample is a body fluid sample, a tissue sample, a cell colony sample, a single cell sample or a cell culture sample. Said biological samples may be mixed or pooled, e.g. the biological sample may be a mixture of a body fluid sample and a tissue sample or a mixture of a body fluid sample and a cell culture sample.

More preferably, the tissue sample from a patient (e.g. human or animal) is a section or an explant sample. It is preferred that the tissue sample from a patient (e.g. human or animal) has a weight of between 0.1 and 500 mg, more preferably of between 0.5 and 250 mg, and most preferably of between 1 and 50 mg, i.e. 0.1, 0.2, 0.3, 0.4, 0.5, 0.6, 0.7, 0.8, 0.9, 1, 2, 2.5, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg.

It is also preferred that the cell sample (e.g. cell colony sample or cell culture sample) from a patient (e.g. human or animal) consists of between 10² and 10¹⁰ cells, more preferably of between 10³ and 10⁷ cells, and most preferably of between 10⁴ and 10⁶ cells, i.e. 10², 10³, 10⁴, 10⁵, 10⁶, 10⁷, 10⁸, 10⁹, or 10¹⁰ cells.

More preferably, the body fluid sample is a blood, urine, sputum, breast milk, cerebrospinal fluid, cerumen (earwax), endolymph, perilymph, gastric juice, mucus, peritoneal fluid, pleural fluid, saliva, sebum (skin oil), semen, sweat, tears, vaginal secretion, or vomit sample including components or fractions thereof.

It is preferred that the body fluid sample from a subject (e.g. human or animal) has a volume of between 0.1 and 20 ml, more preferably of between 0.5 and 10 ml, more preferably between 1 and 8 ml and most preferably between 2 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

Most preferably, the blood sample from a patient (e.g. human or animal) is whole blood or a blood fraction such as serum or plasma. It is also particularly preferred to use blood cells also known as hemopoietic cells. The term “hemopoietic cells” refers to mature cell types and their immature precursors that are identifiable either by morphology or, mostly, by a distinct pattern of cell surface markers. Said term is used to distinguish these cells from other cell types found in the body and also includes T-cells and distinctive subsets, which are the only hematopoietic cells that are not generated in the bone marrow. Said blood cells may be erythrocytes, leukocytes and/or thrombocytes. Peripheral blood mononuclear cells (PBMCs) such as lymphocytes, monocytes or macrophages may also be used.

It is preferred that the blood sample from a patient (e.g. human or animal) has a volume of between 0.1 and 20 ml, more preferably of between 0.5 and 10 ml, and most preferably of between 1 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

The inventors of the present invention surprisingly found that miRNAs are not only present in a blood sample but also that miRNAs remain stable and that, thus, blood miRNAs can be used as biomarkers for detecting and/or prognosis of a lung disease (e.g. sarcoidosis, lung cancer, and/or COPD) in patients (e.g. humans or animals). Furthermore, the use of blood samples in the method of the present invention for detection and/or prognosis of a lung disease has a number of advantage, for example, blood miRNAs have a high sensitivity, blood is relatively easy to obtain and even can be collected via routine physical examination, the costs for detection are low, and the samples can easily be preserved (e.g. at −20° C.). Further, blood circulates to all tissues in the body and, therefore, blood is able to reflect the physiological pathology of the whole organism. Accordingly, the detection of blood miRNAs is an indicator of individual's health and, thus, is an indication whether an individual suffers from a lung disease. Furthermore, this method can widely be used in general screening for a lung disease.

It should be noted that the determination of an expression profile of one or more miRNA(s) in a blood sample from a patient (e.g. human or animal) is possible, (i) as the miRNAs, preferably the miRNAs disclosed herein, are expressed within and/or among cells or tissues and are subsequently released/transferred into circulating blood and/or (ii) as the miRNAs, preferably the miRNAs disclosed herein, are directly expressed in hemopoietic cells, also known as blood cells, e.g. erythrocytes, leukocytes and/or thrombocytes. Thus, the term “determining an expression profile of miRNAs in a blood sample” does not solely mean that the miRNAs are actually expressed in blood. Said miRNAs may be expressed within and/or among surrounding cells or tissues and may be subsequently released/transferred into circulating blood and/or said miRNAs may be directly expressed in blood, namely in blood cells, e.g. peripheral blood mononuclear cells (PBMCs). Accordingly, the expression levels of miRNAs which are preferably determined in a blood sample indirectly represent the expression levels of said miRNAs in the cells or tissues from which they originate and/or directly represent the expression levels of miRNAs in blood cells.

It is also preferred that the urine sample from a patient (e.g. human or animal) has a volume of between 0.1 and 20 ml, more preferably of between 0.5 and 15 ml, and most preferably of between 3 and 12 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

The miRNA expression profiles may be generated by any convenient means for determining miRNA expression levels (see below) and allow the analysis of differential miRNA expression levels between samples, for example, between a sample of a patient and (a) sample(s) of (a) control subject(s), e.g. between a sample of a patient and a sample of a subject known not to suffer form a lung disease such as sarcoidosis, lung cancer, and/or COPD (i.e. being healthy), or between a sample of a patient and a sample of a subject known to suffer from a lung disease such as sarcoidosis, lung cancer, and/or COPD (i.e. being diseased). Thereby, generally, each miRNA is represented by a numerical value. The higher the value of an individual miRNA, the higher is the expression level of said miRNA, or the lower the value of an individual miRNA, the lower is the expression level of said miRNA. The miRNA expression profile may include expression data for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 miRNAs, wherein the nucleotide sequence of said miRNA or the nucleotide sequences of said miRNAs may be selected from the group consisting of SEQ ID NOs: 1 to 391.

The term “differential expression” of miRNAs as used herein, means qualitative and/or quantitative differences in the temporal and/or local miRNA expression patterns, e.g. within and/or among cells, tissues, or within blood. Thus, a differentially expressed miRNA may qualitatively have its expression altered, including an activation or inactivation in, for example, normal tissue versus diseased tissue. The difference in miRNA expression may also be quantitative, e.g. in that expression is modulated, i.e. either up-regulated, resulting in an increased amount of miRNA, or down-regulated, resulting in a decreased amount of miRNA. The degree to which miRNA expression differs need only be large enough to be quantified via standard characterization techniques, e.g. quantitative hybridization of miRNA, labeled miRNA, or amplified miRNA, quantitative PCR (qPCR) such as real time quantitative PCR (qRT-PCR), ELISA for quantitation, next generation sequencing and the like.

The one or more miRNAs having a nucleic acid sequence selected from the group consisting of SEQ ID NOs: 1 to 391 are known to be differentially expressed between subject being healthy and subject having a lung disease (e.g. sarcoidosis, lung cancer, and/or COPD) as well as between subjects having a lung disease (e.g. sarcoidosis, lung cancer and/or COPD) (see FIG. 18). For example, an increase in expression of one or more miRNAs having a nucleic acid sequence selected from the group consisting of SEQ ID NOs: 1 to 391 and/or a decrease in expression of one or more miRNAs having a nucleic acid sequence selected from the group consisting of SEQ ID NOs: 1 to 391 may be indicative for healthiness, sarcoidosis, lung cancer, and/or COPD (see for example median intensity of the miRNAs (g1 and g2) obtained from microarray analysis in FIGS. 1, 13 and 14). In this respect, it should be noted that the same miRNA may be up-regulated in subjects known to suffer from sarcoidosis and down-regulated in subjects known to suffer from lung cancer compared to subjects known to be healthy. It is also possible that the same miRNA may be up-regulated in subjects known to suffer from COPD and in subject known to suffer from lung cancer compared to subjects known to be healthy, or down-regulated in subject known to suffer from COPD and in subjects known to suffer from lung cancer compared to subjects known to be healthy, but that the grade of up-regulation or the grade of down-regulation is different.

As mentioned above, an expression profile of one or more miRNAs, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 miRNAs, in a biological sample from a patient is determined in the step (i) of the method of the present invention, wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

Preferably, the nucleotide sequence of said miRNA or the nucleotide sequences of said miRNAs in step (i) of the method of the present invention is (are) selected from the group consisting of SEQ ID NOs: 1 to 231 (see lists 1, 2, 4 and 7 of FIG. 18), wherein the comparison and/or application in step (ii) of the method of the present invention allow(s) for the diagnosis and/or prognosis of sarcoidosis.

In this respect it is particularly preferred that the nucleotide sequence of said miRNA or the nucleotide sequences of said miRNAs in step (i) of the method of the present invention is (are) selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (see list 1 of FIG. 18), wherein the comparison and/or application in step (ii) allow(s) for the diagnosis and/or prognosis of sarcoidosis. It is further particularly preferred that the nucleotide sequences of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 miRNAs in step (i) of the method of the present invention are selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (see list 1 of FIG. 18), wherein the comparison and/or application in step (ii) allow(s) for the diagnosis and/or prognosis of sarcoidosis.

More preferably, the nucleotide sequence of said miRNA or the nucleotide sequences of said miRNAs in step (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18), wherein the comparison and/or application in step (ii) allow(s) for differential diagnosis (of sarcoidosis) with respect to lung cancer and/or COPD.

Most preferably,
(i) the nucleotide sequence of said miRNA or the nucleotide sequences of said miRNAs in step (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (see list 2 of FIG. 18), wherein the comparison and/or application in step (ii) allow(s) for differential diagnosis (of sarcoidosis) with respect to lung cancer,
(ii) the nucleotide sequence of said miRNA or the nucleotide sequences of said miRNAs in step (i) is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18), wherein the comparison and/or application in step (ii) allow(s) for differential diagnosis (of sarcoidosis) with respect to lung cancer and COPD, or
(iii) the nucleotide sequence of said miRNA or the nucleotide sequences of said miRNAs in step (i) is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18), wherein the comparison and/or application in step (ii) allow(s) for differential diagnosis (of sarcoidosis) with respect to COPD.

In preferred embodiments, the nucleotide sequences of said miRNAs are the nucleotide sequences according to Set No. 1 to 42 (i.e. SH-1 to SH-42) of FIG. 15.

Preferably, the nucleotide sequence of said miRNA or the nucleotide sequences of said miRNAs in step (i) is (are) selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, 329 (see list 3 of FIG. 18), 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18), wherein the comparison and/or application in step (ii) allow(s) for the diagnosis and/or prognosis of lung cancer.

In this respect it is particularly preferred that the nucleotide sequence of said miRNA or the nucleotide sequences of said miRNAs is (are) selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, and 329 (see list 3 of FIG. 18), wherein the comparison and/or application in step (ii) allow(s) for the diagnosis and/or prognosis of lung cancer. It is further particularly preferred that the nucleotide sequences of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 miRNAs in step (i) of the method of the present invention are selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, and 329 (see list 3 of FIG. 18), wherein the comparison and/or application in step (ii) allow(s) for the diagnosis and/or prognosis of lung cancer.

More preferably, the nucleotide sequence of said miRNA or the nucleotide sequences of said miRNAs in step (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18), wherein the comparison and/or application in step (ii) allow(s) for differential diagnosis (of lung cancer) with respect to sarcoidosis and/or COPD.

Most preferably,
(i) the nucleotide sequence of said miRNA or the nucleotide sequences of said miRNAs in step (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (see list 2 of FIG. 18), wherein the comparison and/or application in step (ii) allow(s) for differential diagnosis (of lung cancer) with respect to sarcoidosis,
(ii) the nucleotide sequence of said miRNA or the nucleotide sequences of said miRNAs in step (i) is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18), wherein the comparison and/or application in step (ii) allow(s) for differential diagnosis (of lung cancer) with respect to sarcoidosis and COPD, or
(iii) the nucleotide sequence of said miRNA or the nucleotide sequences of said miRNAs is (are) selected from the group consisting of SEQ ID NOs: 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18), wherein the comparison and/or application in step (ii) allow(s) for differential diagnosis (of lung cancer) with respect to COPD.

In preferred embodiments, the nucleotide sequences of said miRNAs are the nucleotide sequences according to Set No. 1 to 42 (i.e. SL-1 to SL-42) of FIG. 16.

Preferably, the nucleotide sequence of said miRNA or the nucleotide sequences of said miRNAs in step (i) is (are) selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391 (see list 6 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, 231 (see list 7 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18), wherein the comparison and/or application in step (ii) allow(s) for the diagnosis and/or prognosis of COPD.

In this respect it is particularly preferred that the nucleotide sequence of said miRNA or the nucleotide sequences of said miRNAs in step (i) is (are) selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, and 391 (see list 6 of FIG. 18), wherein the comparison and/or application in step (ii) allow(s) for the diagnosis and/or prognosis of COPD. It is further particularly preferred that that the nucleotide sequences of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 miRNAs in step (i) of the method of the present invention are selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, and 391 (see list 6 of FIG. 18), wherein the comparison and/or application in step (ii) allow(s) for the diagnosis and/or prognosis of COPD.

More preferably, the nucleotide sequence of said miRNA or the nucleotide sequences of said miRNAs in step (i) is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, 231 (see list 7 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18), wherein the comparison and/or application in step (ii) allow(s) for differential diagnosis (of COPD) with respect to sarcoidosis and/or lung cancer.

Most preferably,
(i) the nucleotide sequence of said miRNA or the nucleotide sequences of said miRNAs in step (i) is (are) selected from SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18), wherein the comparison and/or application in step (ii) allow(s) for differential diagnosis (of COPD) with respect to sarcoidosis,
(ii) the nucleotide sequence of said miRNA or the nucleotide sequences of said miRNAs is (are) selected from SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18)), wherein the comparison and/or application in step (ii) allow(s) for differential diagnosis (of COPD) with respect to sarcoidosis and lung cancer, or
(iii) the nucleotide sequence of said miRNA or the nucleotide sequences of said miRNAs is (are) selected from SEQ ID NOs: 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18), wherein the comparison and/or application in step (ii) allow(s) for differential diagnosis (of COPD) with respect to lung cancer.

In preferred embodiments, the nucleotide sequences of said miRNAs are the nucleotide sequences according to Set No. 1 to 42 (i.e. SC-1 to SC-42) of FIG. 17.

With respect to the above mentioned embodiments of the second aspect of the present invention, it is preferred that the nucleotide sequences of the miRNAs have (i) SEQ ID NO: 2 and SEQ ID NO: 3, (ii) SEQ ID NO: 3 and SEQ ID NO: 4, (iii) SEQ ID NO: 5, SEQ ID NO: 6, and SEQ ID NO: 7, (iv) SEQ ID NO: 8, SEQ ID NO: 9, and SEQ ID NO: 10, (v) SEQ ID NO: 11, SEQ ID NO: 12, and SEQ ID NO: 13, (vi) SEQ ID NO: 14, SEQ ID NO: 15, and SEQ ID NO: 16, (vii) SEQ ID NO: 17, SEQ ID NO: 18, SEQ ID NO: 19, and SEQ ID NO: 20, (viii) SEQ ID NO: 21, SEQ ID NO: 22, SEQ ID NO: 23, and SEQ ID NO: 24, (ix) SEQ ID NO: 25, SEQ ID NO: 26, SEQ ID NO: 27, SEQ ID NO: 28, SEQ ID NO: 29, and SEQ ID NO: 30, (x) SEQ ID NO: 31, SEQ ID NO: 32, SEQ ID NO: 33, SEQ ID NO: 34, SEQ ID NO: 35, and SEQ ID NO: 36, or (xi) SEQ ID NO: 37, SEQ ID NO: 38, SEQ ID NO: 39, SEQ ID NO: 40. Said miRNAs (miRNA combinations) may also be further combined with each other, e.g. (i) with (ii), (ii) with (iii), (iii) with (iv), (iv) with (v), (v) with (vi), (vi) with (vii), or any other possible combination.

It is also preferred that the nucleotide sequences of the miRNAs have (i) SEQ ID NO: 1 and SEQ ID NO: 2, (ii) SEQ ID NO: 1 and SEQ ID NO: 3, (iii) SEQ ID NO: 1 and SEQ ID NO: 4, (iv) SEQ ID NO: 4 and SEQ ID NO: 5, (v) SEQ ID NO: 5 and SEQ ID NO: 6, (vi) SEQ ID NO: 6 and SEQ ID NO: 7, (vii) SEQ ID NO: 7 and SEQ ID NO: 8, (viii) SEQ ID NO: 8 and SEQ ID NO: 9, (ix) SEQ ID NO: 9 and SEQ ID NO. 10, (x) SEQ ID NO: 10 and SEQ ID NO: 11, or (xi) SEQ ID NO: 11 and SEQ ID NO: 12. Said miRNAs (miRNA combinations) may also be further combined with each other, e.g. (i) with (ii), (ii) with (iii), (iii) with (iv), (iv) with (v), (v) with (vi), (vi) with (vii), or any other possible combination. It is also preferred that the nucleotide sequences of the miRNAs have (i) SEQ ID NO: 1 to SEQ ID NO: 3, (ii) SEQ ID NO: 2 to SEQ ID NO: 4, (iii) SEQ ID NO: 3 to SEQ ID NO: 5, (iv) SEQ ID NO: 4 to SEQ ID NO: 6, (v) SEQ ID NO: 5 to SEQ ID NO: 7, (vi) SEQ ID NO: 6 to SEQ ID NO: 8, (vii) SEQ ID NO: 7 to SEQ ID NO: 9, (viii) SEQ ID NO: 8 to SEQ ID NO: 10, (ix) SEQ ID NO: 9 to SEQ ID NO: 11, (x) SEQ ID NO: 10 to SEQ ID NO: 12, or (xi) SEQ ID NO: 11 to SEQ ID NO: 13. Said miRNAs (miRNA combinations) may also be further combined with each other, e.g. (i) with (ii), (ii) with (iii), (iii) with (iv), (iv) with (v), (v) with (vi), (vi) with (vii), or any other possible combination.

It is particularly preferred that the nucleotide sequences of the miRNAs have (i) SEQ ID NO: 1, SEQ ID NO: 2, SEQ ID NO: 3, SEQ ID NO: 4, SEQ ID NO: 68, SEQ ID NO: 5, SEQ ID NO: 6, SEQ ID NO: 7, SEQ ID NO: 69, and SEQ ID NO: 8, (ii) SEQ ID NO: 68, SEQ ID NO: 5, SEQ ID NO: 6, SEQ ID NO: 7, SEQ ID NO: 69, SEQ ID NO: 8, SEQ ID NO: 70, SEQ ID NO: 105, SEQ ID NO: 9, and SEQ ID NO: 10, (iii) SEQ ID NO: 8, SEQ ID NO: 70, SEQ ID NO: 105, SEQ ID NO: 9, SEQ ID NO: 10, SEQ ID NO: 11, SEQ ID NO: 12, SEQ ID NO: 13, SEQ ID NO: 14, and SEQ ID NO: 114, (iv) SEQ ID NO: 68, SEQ ID NO: 1, SEQ ID NO: 4, SEQ ID NO: 69, SEQ ID NO: 3, SEQ ID NO: 5, SEQ ID NO: 8, SEQ ID NO: 70, SEQ ID NO: 7, and SEQ ID NO: 6, (v) SEQ ID NO: 3, SEQ ID NO: 5, SEQ ID NO: 8, SEQ ID NO: 70, SEQ ID NO: 7, SEQ ID NO: 6, SEQ ID NO: 12, SEQ ID NO: 9, SEQ ID NO: 105, and SEQ ID NO: 107, or (vi) SEQ ID NO: 6, SEQ ID NO: 12, SEQ ID NO: 9, SEQ ID NO: 105, SEQ ID NO: 107, SEQ ID NO: 71, SEQ ID NO: 17, SEQ ID NO: 159, SEQ ID NO: 19, and SEQ ID NO: 106. Said miRNAs (miRNA combinations) may also be further combined with each other, e.g. (i) with (ii), (ii) with (iii), (iii) with (iv), (iv) with (v), (v) with (vi), (vi) with (vii), or any other possible combination.

In another preferred embodiment, the nucleotide sequence of the (single) miRNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, or is selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20. It is particularly preferred that the nucleotide sequence of the (single) miRNA is selected from the group consisting of SEQ ID NO: 1, SEQ ID NO: 2, SEQ ID NO: 3, SEQ ID NO: 4, and SEQ ID NO: 5.

It should be noted that the miRNAs disclosed herein and mentioned above (at least two miRNAs) may be comprised in a set. For example, said set may comprise, essentially consist of, or consist of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 miRNAs, wherein the nucleotide sequences of said miRNAs are selected from the group consisting of SEQ ID NOs: 1 to 391.

As mentioned above, an expression profile of one or more miRNA(s) in a biological sample from a patient is determined in the first step of the method of the present invention, wherein the nucleotide sequence of said miRNA or the nucleotide sequences of said miRNAs is (are) selected from the group consisting of
(i) a nucleotide sequence according to SEQ ID NOs: 1 to 391,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, i.e. 1, 2, 3, 4, 5, 6, 7, 8, 9, 10, 11, or 12, nucleotides shorter than the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

It is particularly preferred that the nucleotide sequence as defined in (iii) has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity over a continuous stretch of at least 10, 11, 12, 13, 14, 15, 16, 17, 18, 19, 20 or more nucleotides, preferably over the whole length, to the nucleotide sequence of the nucleotide according to (i) or nucleotide fragment according to (ii).

In addition, the nucleotide sequence as defined in (ii) (i.e. nucleotide sequence fragment) or (iii) (i.e. nucleotide sequence variant or nucleotide sequence fragment variant) is only regarded as a nucleotide sequence as defined in (ii) (i.e. nucleotide sequence fragment) or (iii) (i.e. nucleotide sequence variant or nucleotide sequence fragment variant) within the context of the present invention, if it can still be bound, hybridized, recognized, or detected by a polynucleotide (probe) of complementary sequence, e.g. a polynucleotide (probe) which is complementary to the respective nucleotide sequence as defined in (i), through one or more types of chemical bonds, usually through complementary base pairing, usually through hydrogen bond formation under stringent hybridization conditions. The skilled person can readily assess whether a nucleotide sequence as defined in (ii) (i.e. nucleotide sequence fragment) or (iii) (i.e. nucleotide sequence variant or nucleotide sequence fragment variant) can still be bound, hybridized, recognized, or detected by a polynucleotide (probe) of complementary sequence, e.g. a polynucleotide (probe) which is complementary to the respective nucleotide sequence as defined in (i). Suitable assays to determine whether hybridization under stringent conditions still occurs are well known in the art. However, as an example, a suitable assay to determine whether hybridization still occurs comprises the steps of: (a) incubating a nucleotide sequence as defined in (ii) or (iii) labelled with biotin with a polynucleotide (probe) of complementary sequence, e.g. a polynucleotide (probe) which is complementary to the respective nucleotide sequence as defined in (i), wherein the polynucleotide (probe) is attached onto a biochip, under stringent hybridization conditions, (b) washing the biochip to remove unspecific bindings, (c) subjecting the biochip to a detection system, and (c) analyzing whether the nucleotide sequence can still be hybridized or detected by a polynucleotide (probe) of complementary sequence, e.g. a polynucleotide (probe) which is complementary to the respective nucleotide sequence as defined in (i). As a positive control, the respective miRNA as defined in (i) may be used. Preferably stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.; or 6×SSPE, 10% formamide, 0.01%, Tween 20, 0.1×TE buffer, 0.5 mg/ml BSA, 0.1 mg/ml herring sperm DNA, incubating at 42° C. with wash in 0.5×SSPE and 6×SSPE at 45° C.

It is preferred that in step (i) of the method according to the second aspect of the present invention, one or more polynucleotide(s) according to the first aspect of the present invention is (are) used for determining an expression profile of one or more miRNA(s) in a biological sample from a patient. Thus, for example, in step (i) of the method of the second aspect of the present invention, one or more polynucleotides is (are) used for determining an expression profile of one or more miRNA(s) in a biological sample, e.g. blood or urine sample, from a patient, e.g. human or animal, wherein the nucleotide sequence of said miRNA is selected from the group consisting of SEQ ID NOs: 1 to 391, a fragment thereof, and a sequence having at least 80% identity thereto.

As mentioned above, in step (i) of the method of the second aspect of the present invention, an expression profile of one or more miRNA(s) is determined in a biological sample, e.g. blood or urine sample, from a patient, e.g. a human or an animal. The determination may be carried out by any convenient means for determining a RNA expression level or RNA expression levels. For expression profiling, qualitative, semi-quantitative and/or quantitative detection methods may be used. A variety of techniques are well known to the person skilled in the art. It is preferred that the expression profile of the miRNA(s) is determined by nucleic acid hybridization, nucleic acid amplification, polymerase extension, sequencing, mass spectroscopy, or any combination thereof.

Nucleic acid amplification may be performed using real time polymerase chain reaction (RT-PCR) such as real time quantitative polymerase chain reaction (RT qPCR). The real time polymerase chain reaction (RT-PCR) is preferred for the analysis of a single miRNA or a low number of miRNAs (e.g. at least 2 to 50 miRNAs such as 2, 5, 10, 20, 30, 40, or 50 miRNAs). It is particularly suitable for detecting low abundance miRNAs. The real time quantitative polymerase chain reaction (RT qPCR), however, allows the analysis of a single miRNA as well as a high number of miRNAs (e.g. at least 2 to 391 miRNAs such as 2, 10, 20, 30, 40, 50, 60, 70, 80, 90, 100, 200, 300, or 391 miRNAs) comprised in a biological sample from a patient, e.g. one or more miRNAs, wherein the nucleotide sequence of said miRNA or the nucleotide sequences of said miRNAs is (are) selected from the group consisting of SEQ ID NOs: 1 to 391.

The aforesaid real time polymerase chain reaction (RT-PCR) may include the following steps: (i) extracting the total RNA from a biological sample such as tissue, urine or blood (e.g. whole blood, serum, or plasma) sample of a patient such as a human or an animal, and obtaining cDNA samples by RNA reverse transcription (RT) reaction using miRNA-specific primers, or collecting a biological sample such as a urine or blood (e.g. whole blood, serum, or plasma) sample of a patient such as a human or an animal, and conducting reverse transcriptase reaction using miRNA-specific primers with the biological sample such as the urine or blood (e.g. whole blood, serum, or plasma) sample being a buffer so as to prepare cDNA samples, (ii) designing miRNA-specific cDNA forward primers and providing universal reverse primers to amplify the cDNA via polymerase chain reaction (PCR), (iii) adding a fluorescent probe to conduct PCR, and (iv) detecting the miRNA(s) level in the biological sample such as the tissue, urine or blood (e.g. whole blood, serum, or plasma) sample.

A variety of kits and protocols to determine an expression profile by real time polymerase chain reaction (RT-PCR) such as real time quantitative polymerase chain reaction (qRT-PCR) are available. For example, reverse transcription of miRNAs may be performed using the TaqMan MicroRNA Reverse Transcription Kit (Applied Biosystems) according to manufacturer's recommendations. Briefly, miRNA may be combined with dNTPs, MultiScribe reverse transcriptase and the primer specific for the target miRNA. The resulting cDNA may be diluted and may be used for PCR reaction. The PCR may be performed according to the manufacturer's recommendation (Applied Biosystems). Briefly, cDNA may be combined with the TaqMan assay specific for the target miRNA and PCR reaction may be performed using ABI 7300.

Nucleic acid hybridization may be performed using a microarray/biochip or in situ hybridization. In situ hybridization is preferred for the analysis of a single miRNA or a low number of miRNAs (e.g. at least 2 to 50 miRNAs such as 2, 5, 10, 20, 30, 40, or 50 miRNAs).

The microarray/biochip, however, allows the analysis of a single miRNA as well as a high number of miRNAs (e.g. at least 2 to 391 miRNAs such as at least such as 2, 10, 20, 30, 40, 50, 60, 70, 80, 90, 100, 200, 300, or 391 miRNAs) comprised in a biological sample such as blood or tissue sample from a patient such as a human or an animal, e.g. one or more miRNAs, wherein the nucleotide sequence of said miRNA or the nucleotide sequences of said miRNAs is (are) selected from the group consisting of SEQ ID NOs: 1 to 391.

For nucleic acid hybridization, for example, the polynucleotides (probes) according to the present invention with complementarity to the corresponding miRNAs to be detected are attached to a solid phase to generate a microarray/biochip (e.g. 391 polynucleotides (probes) which are complementary to the 391 miRNAs having SEQ ID NOs: 1 to 391). Said microarray/biochip is then incubated with miRNAs, isolated (e.g. extracted) from the biological sample such as blood or tissue sample from a patient such as a human or an animal, which may be labelled, e.g. fluorescently labelled, or unlabelled. Upon hybridization of the labelled miRNAs to the complementary polynucleotide sequences on the microarray/biochip, the success of hybridisation may be controlled and the intensity of hybridization may be determined via the hybridisation signal of the label in order to determine the expression level of each tested miRNA in said biological sample, e.g. blood or tissue sample.

Preferably, (i) the nucleic acid hybridization is performed using a microarray/biochip, or using in situ hybridization, and/or (ii) the nucleic acid amplification is performed using real-time PCR.

Subsequent to the determination of an expression profile (data) of one or more miRNAs as defined above in a biological sample (e.g. blood or tissue sample) from a patient (e.g. human or animal) in step (i) of the method according to the present invention, said expression profile (data) is compared to a reference in step (ii) of the method according to the present invention, wherein the comparison of said expression profile (data) to said reference allows for the diagnosis and/or prognosis of a lung disease such as sarcoidosis, lung cancer, and/or COPD, and/or an algorithm or a mathematical function is applied to said expression profile (data) in step (ii) of the method according to the present invention, wherein the application of said algorithm or mathematical function to said expression profile (data) allows for the diagnosis and/or prognosis of a lung disease such as sarcoidosis, lung cancer, and/or COPD.

The reference may be any reference which allows for the diagnosis and/or prognosis of a lung disease such as sarcoidosis, lung cancer, and/or COPD, e.g. an indicated value or values, and/or the algorithm or mathematical function may be any algorithm or mathematical function which allows for the diagnosis and/or prognosis of a lung disease such as sarcoidosis, lung cancer, and/or COPD.

The term “known (clinical) condition” (biological state or health state), as used herein, means a known status of a subject that can be described by physical, mental or social criteria. It includes so-called “healthy” and “diseased” conditions. For the definition of “healthy” and “diseased” conditions it is referred to the international classification of diseases (ICD) of the WHO (http://www.int/classifications/icd/en/index.html). When the expression profile (data) determined in a patient is compared to a reference of a (at least one) known (clinical) condition or when an algorithm or a mathematical function obtained from a reference of a (at least one) known (clinical) condition is applied to the expression profile (data) determined in a patient according to preferred embodiments of the method of the present invention, said known (clinical) condition may be healthiness or a lung disease such as sarcoidosis, lung cancer or COPD.

Further, for example, when the expression profile (data) determined in a patient is compared to a reference of at least two known (clinical) conditions or when an algorithm or a mathematical function obtained from a reference of at least two known (clinical) conditions is applied to the expression profile (data) determined in a patient according to other preferred embodiments of the method of the present invention, said at least two known (clinical) conditions may be (i) healthiness and sarcoidosis, (ii) healthiness and lung cancer, (iii) healthiness and COPD, (iv) sarcoidosis and lung cancer, (v) sarcoidosis and COPD, or (vi) lung cancer and COPD.

Furthermore, for example, when the expression profile (data) determined in a patient is compared to a reference of at least three known (clinical) conditions or when an algorithm or a mathematical function obtained from a reference of at least three known (clinical) conditions is applied to the expression profile (data) determined in a patient according to other preferred embodiments of the method of the present invention, said at least three known (clinical) conditions may be (i) healthiness, sarcoidosis and lung cancer, (ii) healthiness, lung cancer and COPD, (iii) healthiness, sarcoidosis and COPD, or (iv) sarcoidosis, lung cancer and COPD.

In addition, for example, when the expression profile (data) determined in a patient is compared to a reference of at least four known (clinical) conditions or when an algorithm or a mathematical function obtained from a reference of at least four known (clinical) conditions is applied to the expression profile (data) determined in a patient according to other preferred embodiments of the method of the present invention, said at least four known (clinical) conditions may be healthiness, sarcoidosis, lung cancer and COPD.

It should be noted that the term “reference expression profile (data)”, as used herein, means an expression profile (data) of a (one) miRNA that has a nucleotide sequence that essentially corresponds (is essentially identical), preferably that corresponds (is identical), to the nucleotide sequence of the miRNA of step (i), or an expression profile (data) of (more than one) miRNAs that have nucleotide sequences that essentially correspond (are essentially identical), preferably that correspond (are identical), to the nucleotide sequences of the miRNAs of step (i), but means an expression profile which is obtained from a (control) subject(s) with a known (clinical) condition, e.g. healthiness or a lung disease such as sarcoidosis, lung cancer or COPD.

The terms “essentially correspond(s)” or “essentially identical”, as used in the context (of the second aspect) of the present invention, mean that the miRNA(s) of step (i) and the miRNA(s) of the reference expression profile may slightly differ in their nucleotide sequence, whereas said difference is so marginal that it may still allow for the diagnosis and/or prognosis of a lung disease such as sarcoidosis, lung cancer, and/or COPD. Preferably, the nucleotide sequence(s) of the miRNA(s) of step (i) and the nucleotide sequence(s) of the miRNA(s) of the reference expression profile differ in 1 to 5, more preferably in 1 to 3, and most preferably in 1 to 2 nucleotides, i.e. in 1, 2, 3, 4, or 5 nucleotides. It is preferred that said difference resides in 1 to 5, more preferably 1 to 3, and most preferably 1 to 2 nucleotide mutations, i.e. 1, 2, 3, 4, or 5 nucleotide mutations (e.g. substitutions, additions, insertions, and/or deletions). This is caused by the fact that the miRNAs between the species (e.g. humans and animals) and within the species (e.g. humans or animals) may differ. For example, the miRNA(s) of the reference expression profile may be from the same species (e.g. human) as the miRNA(s) of (i) (e.g. human) or may be from a different species (e.g. human) as the miRNA(s) of (i) (e.g. animal), particularly under the condition that the nucleotide sequences of said miRNAs only differ in 1 to 5 nucleotides (e.g. 1, 2, 3, 4, or 5 nucleotides) (see above). It is preferred that the miRNA(s) of step (i) and the miRNA(s) of the reference expression profile do not differ in their nucleotide sequence, i.e. are identical, and/or that the miRNA(s) of step (i) and the miRNA(s) of the reference expression profile are from the same species (e.g. human or animal). It is particularly preferred that the miRNA(s) of step (i) and the miRNA(s) of the reference expression profile are derived from (control) subjects/patients of the same gender and/or similar age or phase of life.

Preferably, both the reference expression profile and the expression profile of step (i) are determined in the same biological sample, e.g. tissue, urine, or blood sample including a blood serum sample, blood plasma sample, or a blood cell sample (e.g. erythrocytes, leukocytes and/or thrombocytes). It is understood that the reference expression profile is not necessarily obtained from a single (control) subject, e.g. a subject known to be affected by a lung disease such as sarcoidosis, lung cancer or COPD, or a subject known to be not affected by a lung disease such as sarcoidosis, lung cancer or COPD (i.e. healthy), but may be an average reference expression profile of a plurality of (control) subjects, e.g. subjects known to be affected by a lung disease such as sarcoidosis, lung cancer or COPD, or subjects known to be not affected by a lung disease such as sarcoidosis, lung cancer or COPD (i.e. healthy), e.g. at least 2 to 40 subjects, more preferably at least 10 to 25 subjects, and most preferably at least 15 to 20 subjects, i.e. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects. The reference expression profile and the expression profile of step (i) may be obtained from a (control) subject/patient of the same species (e.g. human or animal), or may be obtained from a (control) subject/patient of a different species (e.g. human or animal). Preferably, both the reference expression profile and the expression profile of step (i) are obtained from a (control) subject/patient of the same species (e.g. human or animal), of the same gender (e.g. female or male) and/or of a similar age or phase of life (e.g. infant, young child, juvenile, adult).

Thus, in a preferred embodiment of the method of the present invention, the reference is a reference expression profile (data) of at least one subject, preferably the reference is an average expression profile (data) of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with a (at least one) known clinical condition, wherein the reference expression profile is the profile of a (one) miRNA that has a nucleotide sequence that essentially corresponds (is essentially identical), preferably that corresponds (is identical), to the nucleotide sequence of the miRNA of step (i) or is the profile of (more than one) miRNAs that have nucleotide sequences that essentially correspond (are essentially identical), preferably that correspond (are identical), to the nucleotide sequences of the miRNAs of step (i). More preferably, the known clinical condition is healthiness or a lung disease (e.g. sarcoidosis, lung cancer or COPD). Most preferably, the known clinical condition is selected from the group consisting of healthiness, sarcoidosis, lung cancer, and COPD.

For example, said reference may be a reference expression profile of at least one subject known to be not affected by a lung disease such as sarcoidosis, lung cancer or COPD (i.e. healthy) or known to be affected by a lung disease such as sarcoidosis, lung cancer or COPD (i.e. diseased), wherein the reference expression profile is the profile of a miRNA that has a nucleotide sequence that essentially corresponds (is essentially identical), preferably that corresponds (is identical), to the nucleotide sequence of the miRNA of step (i) or is the profile of miRNAs that have nucleotide sequences that essentially correspond (are essentially identical), preferably that correspond (are identical), to the nucleotide sequences of the miRNAs of step (i).

The comparison of the expression profile of the patient to be diagnosed and/or prognosed to the (average) reference expression profile (data), may then allow for the diagnosis and/or prognosis of a lung disease such as sarcoidosis, lung cancer or COPD (step (ii)).

Considering the above, diagnosing preferably means comparing the expression profile (data) of a patient determined in step (i) to the (average) reference expression profile (data) as mentioned above to decide, if the known clinical condition, which is a lung disease such as sarcoidosis, lung cancer or COPD, or which is no lung disease such as sarcoidosis, lung cancer or COPD (i.e. healthy), is present in said patient. Prognosing preferably means comparing the expression profile (data) of a patient determined in step (i) to the (average) reference expression profile (data) as mentioned above to decide, if the known clinical condition, which is a lung disease such as sarcoidosis, lung cancer or COPD, or which is no lung disease such as sarcoidosis, lung cancer or COPD (i.e. healthy), will likely be present in said patient.

For example, the patient may be diagnosed as not suffering from a lung disease such as sarcoidosis, lung cancer or COPD (i.e. being healthy), or as suffering from a lung disease such as sarcoidosis, lung cancer or COPD (i.e. being diseased). Further, for example, the patient may be prognosed as not developing a lung disease such as sarcoidosis, lung cancer or COPD (i.e. staying healthy), or as developing a lung disease such as sarcoidosis, lung cancer or COPD (i.e. getting diseased).

Diagnosing/prognosing of a lung disease such as sarcoidosis, lung cancer or COPD based on (a) reference expression profile (data) as reference may take place as follows: For instance, (i) if the miRNA(s) of step (i) (e.g. a single miRNA or at least 2 miRNAs) in the expression profile of a patient to be diagnosed for a lung disease such as sarcoidosis, lung cancer or COPD is (are), for example, at least 2 fold higher (up-regulated) compared to said miRNA(s) (e.g. a single miRNA or at least 2 miRNAs) in the reference expression profile of a subject known not to suffer from a lung disease such as sarcoidosis, lung cancer or COPD (i.e. being healthy), the patient tested is diagnosed as suffering from a lung disease such as sarcoidosis, lung cancer or COPD (i.e. being diseased) or prognosed as likely developing a lung disease such as sarcoidosis, lung cancer or COPD (i.e. getting diseased), or (ii) if the miRNA(s) of step (i) (e.g. a single miRNA or at least 2 miRNAs) in the expression profile of a patient to be diagnosed for a lung disease such as sarcoidosis, lung cancer or COPD is (are), for example, at least 2 fold lower (down-regulated) compared to said miRNA(s) (e.g. a single miRNA or a set of at least 2 miRNAs) in the reference expression profile of a subject known not to suffer from a lung disease such as sarcoidosis, lung cancer or COPD (i.e. being healthy), the patient tested is diagnosed as suffering from a lung disease such as sarcoidosis, lung cancer or COPD (i.e. being diseased) or prognosed as likely developing a lung disease such as sarcoidosis, lung cancer or COPD (i.e. getting diseased).

In the converse case, (i) if the miRNA(s) of step (i) (e.g. a single miRNA or at least 2 miRNAs) in the expression profile of a patient to be diagnosed for a lung disease such as sarcoidosis, lung cancer or COPD is (are), for example, not at least 2 fold higher (up-regulated) compared to said miRNA(s) (e.g. a single miRNA or at least 2 miRNAs) in the reference expression profile of a subject known not to suffer from a lung disease such as sarcoidosis, lung cancer or COPD (i.e. being healthy), the patient tested is diagnosed as not suffering from a lung disease such as sarcoidosis, lung cancer or COPD (i.e. being healthy) or prognosed as likely not developing a lung disease such as sarcoidosis, lung cancer or COPD (i.e. staying healthy), or (ii) if the miRNA(s) of step (i) (e.g. a single miRNA or at least 2 miRNAs) in the expression profile of a patient to be diagnosed for a lung disease such as sarcoidosis, lung cancer or COPD is (are), for example, not at least 2 fold lower (down-regulated) compared to said miRNA(s) (e.g. a single miRNA or at least 2 miRNAs) in the reference expression profile of subject known not to suffer from a lung disease such as sarcoidosis, lung cancer or COPD (i.e. being healthy), the patient tested (e.g. human or animal) is diagnosed as not suffering from a lung disease such as sarcoidosis, lung cancer or COPD (i.e. being healthy) or prognosed as likely not developing a lung disease such as sarcoidosis, lung cancer or COPD (i.e. staying healthy).

It should be noted that a patient that is diagnosed as being healthy, i.e. not suffering from a lung disease, may possibly suffer from another disease not tested/known, or a patient that is prognosed as staying healthy, i.e. likely not developing a lung disease, may possibly developing another disease not tested/known (see also definition of the term “patient” above).

Preferably, the algorithm or mathematical function is obtained from a reference expression profile of at least one subject, preferably of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with a (at least one) known clinical condition, wherein the reference expression profile is the profile of a (one) miRNA that has a nucleotide sequence that essentially corresponds (is essentially identical), preferably that corresponds (is identical), to the nucleotide sequence of the miRNA of step (i), or is the profile of (more than one) miRNAs that have nucleotide sequences that essentially correspond (are essentially identical), preferably that correspond (are identical), to the nucleotide sequences of the miRNAs of step (i). More preferably, the known clinical condition is healthiness or a lung disease (e.g. sarcoidosis, lung cancer or COPD). Most preferably, the known clinical condition is selected from the group consisting of healthiness, sarcoidosis, lung cancer, and COPD.

For example, said algorithm or mathematical function may be obtained from a reference expression profile of at least one subject known to be not affected by a lung disease such as sarcoidosis, lung cancer or COPD (i.e. healthy), or known to be affected by a lung disease such as sarcoidosis, lung cancer or COPD (i.e. diseased), wherein the reference expression profile is the profile of a miRNA that has a nucleotide sequence that essentially corresponds (is essentially identical), preferably that corresponds (is identical), to the nucleotide sequence of the miRNA of step (i) or is the profile of miRNAs that have nucleotide sequences that essentially correspond (are essentially identical), preferably that correspond (are identical), to the nucleotide sequences of the miRNAs of step (i).

It is particularly preferred that the algorithm or mathematical function is obtained from a reference expression profile of at least one subject, preferably of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with at least one known clinical condition, wherein the reference expression profile is the profile of a miRNA that has a nucleotide sequence that essentially corresponds (is essentially identical), preferably that corresponds (is identical), to the nucleotide sequence of the miRNA of step (i) or is the profile of miRNAs that have nucleotide sequences that essentially correspond (are essentially identical), preferably that correspond (are identical), to the nucleotide sequences of the miRNAs of step (i). More preferably, the at least one known clinical condition is healthiness or a lung disease (e.g. sarcoidosis, lung cancer or COPD). Most preferably, the at least one known clinical condition is selected from the group consisting of healthiness, sarcoidosis, lung cancer, and COPD.

It is also particularly preferred that the algorithm or mathematical function is obtained from reference expression profiles of at least two subjects, preferably of at least 3 to 40 subjects, more preferably of at least 10 to 25 subjects, and most preferably of at least 15 to 20 subjects, i.e. of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with at least two known clinical conditions, preferably 2 to 4 (e.g. 2, 3, or 4) known clinical conditions, wherein the reference expression profiles are the profiles of a miRNA that has a nucleotide sequence that essentially corresponds (is essentially identical), preferably that corresponds (is identical) to the nucleotide sequence of the miRNA of step (i), or are the profiles of miRNAs that have nucleotide sequences that essentially correspond (are essentially identical), preferably that correspond (are identical), to the nucleotide sequences of the miRNAs of step (i). Preferably, the at least two known clinical conditions are selected from the group consisting of healthiness, sarcoidosis, lung cancer, and COPD.

More preferably, the algorithm or mathematical function is obtained from (a) reference expression profile(s) of
(i) at least one subject with the known clinical condition of sarcoidosis,
(ii) at least one subject with the known clinical condition of healthiness,
(iii) at least one subject with the known clinical condition of lung cancer, and/or
(iv) at least one subject with the known clinical condition of COPD,
   wherein the reference expression profile(s) is (are) the profile(s) of a miRNA that has a nucleotide sequence that essentially corresponds (is essentially identical), preferably that corresponds (is identical), to the nucleotide sequence of the miRNA of step (i), or is (are) the profile(s) of miRNAs that have nucleotide sequences that essentially correspond (are essentially identical), preferably that correspond (are identical), to the nucleotide sequences of the miRNAs of step (i).

For example, the algorithm or mathematical function may be obtained from a reference expression profile of
(i) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, healthiness, lung cancer, or COPD,
(ii) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, lung cancer, or COPD,
(iii) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer or COPD,
(iv) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
(v) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer,
(vi) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
(vii) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
(viii) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD, or
(ix) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
   wherein the reference expression profile(s) is (are) the profile(s) of a miRNA that has a nucleotide sequence that essentially corresponds (is essentially identical), preferably that corresponds (is identical), to the nucleotide sequence of the miRNA of step (i), or is (are) the profile(s) of miRNAs that have nucleotide sequences that essentially correspond (are essentially identical), preferably that correspond (are identical), to the nucleotide sequences of the miRNAs of step (i).

Preferably, the above mentioned algorithm or mathematical function is obtained from reference expression profiles of the same number of (control) subjects (e.g. subjects known to be healthy or diseased). For example, the algorithm or mathematical function may be obtained from reference expression profiles of 10 subjects known to suffer from a lung disease such as sarcoidosis, lung cancer or COPD (positive control) and 10 subjects known not to suffer from a lung disease such as sarcoidosis, lung cancer or COPD (negative control). The algorithm or mathematical function may also be obtained from reference expression profiles of 20 subjects known to suffer from a lung disease such as sarcoidosis, lung cancer or COPD (positive control) and 20 subjects known not to suffer from a lung disease such as sarcoidosis, lung cancer or COPD (negative control).

It is preferred that the algorithm or mathematical function is obtained using a machine learning approach. Machine learning approaches may include but are not limited to supervised or unsupervised analysis: classification techniques (e.g. naïve Bayes, Linear Discriminant Analysis, Quadratic Discriminant Analysis Neural Nets, Tree based approaches, Support Vector Machines, Nearest Neighbour Approaches), Regression techniques (e.g. linear Regression, Multiple Regression, logistic regression, probit regression, ordinal logistic regression ordinal probit regression, Poisson Regression, negative binomial Regression, multinomial logistic Regression, truncated regression), Clustering techniques (e.g. k-means clustering, hierarchical clustering, PCA), Adaptations, extensions, and combinations of the previously mentioned approaches.

The inventors of the present invention surprisingly found that the application of a machine learning approach (e.g. t-test, AUC, support vector machine, hierarchical clustering, or k-means) leads to the obtainment of an algorithm or a mathematical function that is trained by the reference expression profile(s) (data) mentioned above and that this allows (i) a better discrimination between the known clinical conditions (statistical classes) (e.g. 2, 3 or 4 known clinical conditions or 2, 3 or 4 known statistical classes) or (ii) a better decision, whether a (at least one) known clinical condition (statistical class) (e.g. 1, 2, 3 or 4 known clinical condition(s) or 1, 2, 3 or 4 known statistical classe(s)) is present. In this way, the performance for diagnosing/prognosing of patients suffering from a lung disease such as sarcoidosis, lung cancer and/or COPD can be increased (see also experimental section for details).

Preferably, the machine learning approach involves the following steps:
(i) inputting the reference expression profile of at least one subject, preferably of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with a (at least one) known clinical condition, and
(ii) computing an algorithm or a mathematical function based on said reference expression profile that is suitable to decide if the known clinical condition is present or will likely be present in said patient.

More preferably, the known clinical condition may be healthiness or a lung disease (e.g. sarcoidosis, lung cancer or COPD). Most preferably, the known clinical condition is selected from the group consisting of healthiness, sarcoidosis, lung cancer, and COPD.
It is particularly preferred that the machine learning approach involves the following steps:
(i) inputting the reference expression profile of at least one subject, preferably of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with at least one known clinical condition, and
(ii) computing an algorithm or a mathematical function based on said reference expression profile that is suitable to decide if the at least one known clinical condition is present or will likely be present in said patient.

More preferably, the at least one known clinical condition is healthiness or a lung disease (e.g. sarcoidosis, lung cancer or COPD). Most preferably, the at least one known clinical condition is selected from the group consisting of healthiness, sarcoidosis, lung cancer, and COPD.
It is also particularly preferred that the machine learning approach involves the following steps:
(i) inputting the reference expression profile of at least two subjects, preferably of at least 3 to 40 subjects, more preferably of at least 10 to 25 subjects, and most preferably of at least 15 to 20 subjects, i.e. of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with at least two known clinical conditions, preferably 2 to 4 (e.g. 2, 3, or 4) known clinical conditions, and
(ii) computing an algorithm or a mathematical function based on said reference expression profile that is suitable to decide if the at least two known clinical conditions are present or will likely be present in said patient.

Preferably, the at least two known clinical conditions are selected from the group consisting of healthiness, sarcoidosis, lung cancer, and COPD.

More preferably, the machine learning approach involves the following steps:
(i) inputting the reference expression profile(s) of

(ia) at least one subject with the known clinical condition of sarcoidosis,

(ib) at least one subject with the known clinical condition of healthiness,

(ic) at least one subject with the known clinical condition of lung cancer, and/or

(id) at least one subject with the known clinical condition of COPD, and
(ii) computing an algorithm or a mathematical function based on said reference expression profile(s) that is suitable to decide if the known clinical condition(s) selected from the group consisting of sarcoidosis, healthiness, lung cancer and COPD is (are) present or will likely be present in said patient.

For example, the machine learning approach involves the following steps:
(i) inputting the reference expression profile(s) of
   (ia) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, healthiness, lung cancer, or COPD,
   (ib) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, lung cancer, or COPD,
   (ic) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer or COPD,
   (id) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
   (ie) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer,
   (if) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
   (ig) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD,
   (ih) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD, or
   (ij) at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of healthiness, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of sarcoidosis, at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of lung cancer, and at least one subject, preferably of at least 2 to 40 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with the known clinical condition of COPD, and
(ii) computing an algorithm or a mathematical function based on said reference expression profile(s) that is suitable to decide if the known clinical condition(s) selected from the group consisting of sarcoidosis, healthiness, lung cancer and COPD is (are) present or will likely be present in said patient.

It is, for example, possible that the known clinical condition sarcoidosis, healthiness, lung cancer or COPD is present or will likely be present in said patient. It is, for example, also possible that the known clinical conditions sarcoidosis and lung cancer, sarcoidosis and COPD, lung cancer and COPD, or sarcoidosis, lung cancer and COPD are present or will likely be present in said patient.

The application of the algorithm or mathematical function as mentioned above to the expression profile of the patient to be diagnosed and/or prognosed may then allow for diagnosing and/or prognosing of a lung disease such as sarcoidosis, lung cancer, and/or COPD. It may also allow (i) for differentially diagnosing sarcoidosis with respect to lung cancer and/or COPD, (ii) for differentially diagnosing lung cancer with respect to sarcoidosis and/or COPD, or (iii) for differentially diagnosing COPD with respect to sarcoidosis and/or lung cancer (see above and FIG. 18). In this respect it should be noted that the differential diagnosis may lead to the result that no differentiation between the different diseases/conditions is possible as the patient tested also suffers from said disease/condition or said diseases/conditions. This applies to all embodiments of the present invention, where a differential diagnosis is carried out. Thus, for example, (i) the differential diagnosis of sarcoidosis with respect to lung cancer and/or COPD may lead to the result that the patient also suffers from (ia) lung cancer, (ib) COPD, or (ic) lung cancer and COPD, (ii) the differential diagnosis of lung cancer with respect to sarcoidosis and/or COPD may lead to the result that the patient also suffers from (iia) sarcoidosis, (iib) COPD, or (iic) sarcoidosis and COPD, or (iii) the differential diagnosis of COPD with respect to sarcoidosis and/or lung cancer may lead to the result that the patient also suffers from (iiia) sarcoidosis, (iiib) lung cancer, or (iiic) sarcoidosis and lung cancer.

Considering the above, diagnosing preferably means applying the algorithm or mathematical function as mentioned above to the expression profile of a patient to decide, if the known clinical condition, which is a lung disease such as sarcoidosis, lung cancer or COPD (i.e. diseased condition), or which is no lung disease such as sarcoidosis, lung cancer or COPD (i.e. healthy condition), is present in said patient. More preferably, the known clinical condition is selected from the group consisting of healthiness, sarcoidosis, lung cancer and COPD. Prognosing preferably means applying the algorithm or mathematical function as mentioned above to the expression profile of a patient to decide, if the known clinical condition, which is a lung disease such as sarcoidosis, lung cancer or COPD (i.e. diseased condition), or which is no lung disease such as sarcoidosis, lung cancer or COPD (i.e. healthy condition), will likely be present in said patient. More preferably, the known clinical condition is selected from the group consisting of healthiness, sarcoidosis, lung cancer and COPD.

For example, the patient may be diagnosed as not suffering from a lung disease such as sarcoidosis, lung cancer or COPD (i.e. being healthy), or as suffering from a lung disease such as sarcoidosis, lung cancer or COPD (i.e. being diseased). Further, for example, the patient may be prognosed as not developing a lung disease such as sarcoidosis, lung cancer or COPD (i.e. staying healthy), or as developing a lung disease such as sarcoidosis, lung cancer or COPD (i.e. getting diseased).

Further, considering the above, diagnosing preferably means applying the algorithm or mathematical function as mentioned above to the expression profile of a patient to decide, if the at least one known clinical condition, which is a lung disease such as sarcoidosis, lung cancer or COPD (i.e. diseased condition), or which is no lung disease such as sarcoidosis, lung cancer or COPD (i.e. healthy condition), is present in said patient. More preferably, the at least one known clinical condition is selected from the group consisting of healthiness, sarcoidosis, lung cancer and COPD. Prognosing preferably means applying the algorithm or mathematical function as mentioned above to the expression profile of a patient to decide, if the at least one known clinical condition, which is a lung disease such as sarcoidosis, lung cancer or COPD (i.e. diseased condition), or which is no lung disease such as sarcoidosis, lung cancer or COPD (i.e. healthy condition), will likely be present in said patient. More preferably, the at least one known clinical condition is selected from the group consisting of healthiness, sarcoidosis, lung cancer and COPD.

Furthermore, considering the above, diagnosing preferably means applying the algorithm or mathematical function as mentioned above to the expression profile of a patient to decide/differentiate which of the at least two known clinical conditions (e.g. 2, 3, or 4 known clinical conditions) selected from the group consisting of healthiness, sarcoidosis, lung cancer and COPD are present in said patient. Prognosing preferably means applying the algorithm or mathematical function as mentioned above to the expression profile of a patient to decide/differentiate which of the at least two known clinical conditions (e.g. 2, 3, or 4 known clinical conditions) selected from the group consisting of healthiness, sarcoidosis, lung cancer and COPD will likely be present in said patient.

For example, if the at least two known clinical conditions are sarcoidosis, lung cancer or COPD (i.e. diseased condition) and no lung disease such as sarcoidosis, lung cancer or COPD (i.e. healthy condition), the patient may be diagnosed as suffering from a lung disease such as sarcoidosis, lung cancer or COPD (i.e. being diseased), or as not suffering from a lung disease such as sarcoidosis, lung cancer or COPD (i.e. being healthy). If the at least two known clinical conditions are a lung disease such as sarcoidosis, lung cancer or COPD (i.e. diseased condition) and no lung disease such as sarcoidosis, lung cancer or COPD (i.e. healthy condition), the patient may be prognosed as developing a lung disease such as sarcoidosis, lung cancer or COPD (i.e. getting diseased), or as not developing a lung disease such as sarcoidosis, lung cancer or COPD (i.e. staying healthy).

Diagnosing/prognosing of a lung disease such as sarcoidosis, lung cancer or COPD based on an algorithm or a mathematical function may take place as follows: For instance, if the algorithm or mathematical function, which is obtained from a reference expression profile of at least one subject with the known clinical condition of a lung disease such as sarcoidosis, lung cancer or COPD, is applied to the expression profile (data) of a patient, the patient is classified as suffering from a lung disease such as sarcoidosis, lung cancer or COPD (i.e. being diseased), if the resulting score is below a specified threshold, or if the algorithm or mathematical function, which is obtained from a reference expression profile of at least one subject with the known clinical condition of no lung disease such as sarcoidosis, lung cancer or COPD, is applied to the expression profile (data) of the patient, the patient is classified as not suffering from a lung disease such as sarcoidosis, lung cancer or COPD (i.e. being healthy), if the resulting score is above a specified threshold.

Further, if the algorithm or mathematical function, which is obtained from a reference expression profile of at least one subject with the known clinical condition of a lung disease such as sarcoidosis, lung cancer or COPD and from a reference expression profile of at least one subject with the known clinical condition of no lung disease such as sarcoidosis, lung cancer or COPD, is applied to the expression profile (data) of a patient, the patient is classified as suffering from a lung disease such as sarcoidosis, lung cancer or COPD (i.e. being diseased), if the resulting score is below a specified threshold, and the patient is classified as not suffering from a lung disease such as sarcoidosis, lung cancer or COPD (i.e. being healthy), if the resulting score is above a specified threshold.

Preferably, machine learning approaches are employed to develop/obtain algorithms or mathematical functions for diagnosing and/or prognosing of clinical conditions such as lung diseases, e.g. sarcoidosis, lung cancer, and/or COPD. Support vector machines (SVMs) are a set of related supervised learning methods which are preferably used for classification and regression. For example, given a set of training examples (e.g. reference expression profile(s) of (a) subject(s) with the known clinical condition(s) of a lung disease such as sarcoidosis, lung cancer or COPD, and/or no lung disease such as sarcoidosis, lung cancer or COPD), each marked as belonging to one of one category (e.g. condition of a lung disease such as sarcoidosis, lung cancer or COPD, or no lung disease such as sarcoidosis, lung cancer or COPD) or to one of two categories (e.g. condition of a lung disease such as sarcoidosis, lung cancer or COPD, and no lung disease such as sarcoidosis, lung cancer o COPD), an SVM algorithm builds a model that predicts whether a new example (e.g. sample from a patient to be tested) falls into one category or the other (e.g. condition of a lung disease such as sarcoidosis, lung cancer or COPD, or no lung disease such as sarcoidosis, lung cancer or COPD). A SVM model is a representation of the training examples (e.g. reference expression profile(s) of (a) subject(s) with the known clinical condition of a lung disease such as sarcoidosis, lung cancer or COPD, and/or no lung disease such as sarcoidosis, lung cancer or COPD) as points in space, mapped so that the training examples of the separate categories (e.g. condition of a lung disease such as sarcoidosis, lung cancer or COPD, or no lung disease such as sarcoidosis, lung cancer or COPD) are divided by a clear gap that is as wide as possible. New examples (e.g. sample from a patient to be tested) are then mapped into that same space and predicted to belong to a category based on which side of the gap they fall on (e.g. lung disease such as sarcoidosis, lung cancer or COPD, or no lung disease such as sarcoidosis, lung cancer or COPD). More formally, a support vector machine constructs a hyperplane or set of hyperplanes in a high or infinite dimensional space, which can be used for classification, regression or other tasks. A good separation is achieved by the hyperplane that has the largest distance to the nearest training data points of any class (so-called functional margin), since in general the larger the margin the lower the generalization error of the classifier.

Classifying data is a preferred task in machine learning. For example, considering some given data points each belong to one (e.g. a lung disease such as sarcoidosis, lung cancer or COPD, or no lung disease such as sarcoidosis, lung cancer or COPD) of two classes (e.g. a lung disease such as sarcoidosis, lung cancer or COPD, and no lung disease such as sarcoidosis, lung cancer or COPD), the goal is to decide which class a new data point (e.g. achieved from a patient) will be in. In the preferred case of support vector machines, a data point is viewed as a p-dimensional vector (a list of p numbers), and the question is, whether it is possible to separate such points with a p-1-dimensional hyperplane. This is called a linear classifier. There are many hyperplanes that might classify the data. One reasonable choice as the best hyperplane is the one that represents the largest separation, or margin, between the two classes (e.g. condition of a lung disease such as sarcoidosis, lung cancer or COPD, or no lung disease such as sarcoidosis, lung cancer or COPD). Thus, the hyperplane should be chosen so that the distance from it to the nearest data point on each side is maximized. If such a hyperplane exists, it is known as the maximum-margin hyperplane and the linear classifier it defines is known as a maximum margin classifier.

The formalization of the support vector machine is exemplary summarised below. For example, given are training data [symbol] (e.g. reference expression profile(s) of (a) subject(s) with the known clinical condition of a lung disease such as sarcoidosis, lung cancer or COPD, and/or no lung disease such as sarcoidosis, lung cancer or COPD), a set of n subject samples (e.g. subjects known to suffer from a lung disease such as sarcoidosis, lung cancer or COPD, and/or no lung disease such as sarcoidosis, lung cancer or COPD) of the form

[symbol]

={(xᵢ,cᵢ)|xᵢε [symbol] , cᵢε{−1,1,}}ᵢ₌₁ⁿ

where the cᵢ is either 1 or −1, indicating the class labels (e.g. a lung disease such as sarcoidosis, lung cancer or COPD, and/or no lung disease such as sarcoidosis, lung cancer or COPD) to which the miRNA biomarker intensities xᵢ belongs. Each xᵢ is a p-dimensional real vector (with p being the number of miRNA biomarkers). The task is know to find the maximum-margin hyperplane that divides the points having cᵢ=1 from those having cᵢ=−1. Any hyperplane can be written as the set of points X satisfying

w·x−b=0,

where “·” denotes the dot product. The vector w is a normal vector: it is perpendicular to the hyperplane. The parameter

$$\frac{b}{‖ w ‖}$$

determines the offset of the hyperplane from the origin along the normal vector w.

w and b should be chosen to maximize the margin, or distance between the parallel hyperplanes that are as far apart as possible while still separating the data. These hyperplanes can be described by the equations

w·x−b=1

and

w·x−b=−1

Note that if the training data are linearly separable, the two hyperplanes of the margin can be selected in a way that there are no points between them and then by trying to maximize their distance. For example, by using geometry, the distance between these two hyperplanes is

$$\frac{2}{‖ w ‖} ,$$

so ∥w∥ should be minimized. To also prevent data points falling into the margin, the following constraint should be added: for each i either

w·xᵢ−b≧1
for xᵢ of the first class (e.g. a lung disease)
   or

w·xᵢ−b≦−1
for xᵢ of the second class (e.g. a lung disease)
   This can be rewritten as:

cᵢ(w·xᵢ−b)≧1, for all 1≦i≦n. (1)

This can be put together to get the optimization problem:

### Minimize (in w, b)

∥w∥

subject to (for any i=1, . . . , n)

cᵢ(w·xᵢ−b)≧1.

The optimization problem presented in the preceding section is difficult to solve because it depends on ∥w∥, the norm of w, which involves a square root. It is, however, possible to alter the equation by substituting ∥w∥ with

$$\frac{1}{2} {‖ w ‖}^{2}$$

(the factor of ½ being used for mathematical convenience) without changing the solution (the minimum of the original and the modified equation have the same w and b). This is a quadratic programming (QP) optimization problem. More clearly:
Minimize (in w,b)

$$\frac{1}{2} {‖ w ‖}^{2}$$

subject to (for any i=1, . . . , n)

cᵢ(w·xᵢ−b)≧1.

The previous problem may be expressed by means of non-negative Lagrange multipliers αᵢ as

$${min}_{w , b , α} \{ \frac{1}{2} {‖ w ‖}^{2} - {\sum }_{i = 1}^{n} {α}_{i} [ {c}_{i} ( w \cdot {x}_{i} - b ) - 1 ] \}$$

but this would led to an incorrect result. The reason is the following: suppose that a family of hyperplanes which divide the points can be found; then all cᵢ(w·xᵢ−b)−1≧0. Hence the minimum by sending all αᵢ to +∞ could be found, and this minimum would be reached for all the members of the family, not only for the best one which can be chosen solving the original problem.
Nevertheless the previous constrained problem can be expressed as

$${min}_{w , b} {max}_{α} \{ \frac{1}{2} {‖ w ‖}^{2} - {\sum }_{i = 1}^{n} {α}_{i} [ {c}_{i} ( w \cdot {x}_{i} - b ) - 1 ] \}$$

looking for a saddle point. In doing so all the points which can be separated as cᵢ(w·xᵢ−b)−1>0 do not matter since the corresponding αᵢ must be set to zero.
This problem can be solved by standard quadratic programming techniques and programs. The solution can be expressed by terms of linear combination of the training vectors as

$$w = {\sum }_{i = 1}^{n} {α}_{i} {c}_{i} {x}_{i}$$

Only a few αᵢ will be greater than zero. The corresponding xᵢ are exactly the support vectors, which lie on the margin and satisfy cᵢ(w·xᵢ−b)=1. From this it can be derived that the support vectors also satisfy

w·xᵢ−b=1/cᵢ=cᵢ [symbol] b=w·xᵢ−cᵢ

which allows one to define the offset b. In practice, it is more robust to average over all N_{SV} support vectors:

$$b = \frac{1}{{N}_{SV}} {\sum }_{i = 1}^{{N}_{SV}} ( w \cdot {x}_{i} - {c}_{i} )$$

Writing the classification rule in its unconstrained dual form reveals that the maximum margin hyperplane and therefore the classification task is only a function of the support vectors, the training data that lie on the margin.
Using the fact, that ∥w∥²=w·w and substituting

$$w = {\sum }_{i = 0}^{n} {α}_{i} {c}_{i} {x}_{i} ,$$

it can show shown that the dual of the SVM reduces to the following optimization problem:
Maximize (in αᵢ)

$$\tilde{L} ( α ) = {\sum }_{i = 1}^{n} {α}_{i} - \frac{1}{2} {\sum }_{i , j} {α}_{i} {α}_{j} {c}_{i} {c}_{j} {x}_{i}^{T} {x}_{j} = {\sum }_{i = 1}^{n} {α}_{i} - \frac{1}{2} {\sum }_{i , j} {α}_{i} {α}_{j} {c}_{i} {c}_{j} k ( {x}_{i} , {x}_{j} )$$

subject to (for any i=1, . . . n)

αᵢ≧0,

and to the constraint from the minimization in b

$${\sum }_{i = 1}^{n} {α}_{i} {c}_{i} = 0.$$

Here the kernel is defined by k(xᵢ,xⱼ)=xᵢ·xⱼ.

The α terms constitute a dual representation for the weight vector in terms of the training set:

$$w = {\sum }_{i} {α}_{i} {c}_{i} {x}_{i} .$$

For simplicity reasons, sometimes it is required that the hyperplane passes through the origin of the coordinate system. Such hyperplanes are called unbiased, whereas general hyperplanes not necessarily passing through the origin are called biased. An unbiased hyperplane can be enforced by setting b=0 in the primal optimization problem. The corresponding dual is identical to the dual given above without the equality constraint

$${\sum }_{i = 1}^{n} {α}_{i} {c}_{i} = 0.$$

Transductive support vector machines extend SVMs in that they could also treat partially labeled data in semi-supervised learning. Here, in addition to the training set [symbol] , the learner is also given a set

[symbol]

={xᵢ*|xᵢ*ε [symbol] }ᵢ₌₁^{k}

of test examples to be classified. Formally, a transductive support vector machine is defined by the following primal optimization problem:

### Minimize (in w, b, c*)

$$\frac{1}{2} {‖ w ‖}^{2}$$

subject to (for any i=1, . . . , n and any j=1, . . . , k)

cᵢ(w·xᵢ−b)≧1,

cⱼ*(w·xⱼ*−b)≧1,

and

cⱼ*ε{−1,1}.

Transductive support vector machines were introduced by Vladimir Vapnik in 1998.
SVMs belong to a family of generalized linear classifiers. They can also be considered a special case of Tikhonov regularization. A special property is that they simultaneously minimize the empirical classification error and maximize the geometric margin; hence they are also known as maximum margin classifiers.
In 1995, Corinna Cortes and Vladimir Vapnik suggested a modified maximum margin idea that allows for mislabeled examples. If there exists no hyperplane that can split the “yes” and “no” examples, the Soft Margin method will choose a hyperplane that splits the examples as cleanly as possible, while still maximizing the distance to the nearest cleanly split examples. The method introduces slack variables, ξᵢ, which measure the degree of misclassification of the datum xᵢ

cᵢ(w·xᵢ−b)≧1−ξᵢ 1≦i≦n. (2)

The objective function is then increased by a function which penalizes non-zero εᵢ, and the optimization becomes a trade off between a large margin, and a small error penalty. If the penalty function is linear, the optimization problem becomes:

$${min}_{w , ξ} \{ \frac{1}{2} {‖ w ‖}^{2} + C {\sum }_{i = 1}^{n} {ξ}_{i} \}$$

subject to (for any i=1, . . . n)

cᵢ(w·xᵢ−b)≧1−ξᵢ, ξᵢ≧0.

This constraint in(2) along with the objective of minimizing ∥w∥ can be solved using Lagrange multipliers as done above. One has then to solve the following problem

$${min}_{w , ξ , b} {max}_{α , β} \{ \frac{1}{2} {‖ w ‖}^{2} + C {\sum }_{i = 1}^{n} {ξ}_{i} - {\sum }_{i = 1}^{n} {α}_{i} [ {c}_{i} ( w \cdot {x}_{i} - b ) - 1 + {ξ}_{i} ] - {\sum }_{i = 1}^{n} {β}_{i} {ξ}_{i} \}$$

with αᵢ, βᵢ≧0.
The key advantage of a linear penalty function is that the slack variables vanish from the dual problem, with the constant C appearing only as an additional constraint on the Lagrange multipliers. For the above formulation and its huge impact in practice, Cortes and Vapnik received the 2008 ACM Paris Kanellakis Award. Non-linear penalty functions have been used, particularly to reduce the effect of outliers on the classifier, but unless care is taken, the problem becomes non-convex, and thus it is considerably more difficult to find a global solution.
The original optimal hyperplane algorithm proposed by Vladimir Vapnik in 1963 was a linear classifier. However, in 1992, Bernhard Boser, Isabelle Guyon and Vapnik suggested a way to create non-linear classifiers by applying the kernel trick (originally proposed by Aizerman et al.^{[4]}) to maximum-margin hyperplanes. The resulting algorithm is formally similar, except that every dot product is replaced by a non-linear kernel function. This allows the algorithm to fit the maximum-margin hyperplane in a transformed feature space. The transformation may be non-linear and the transformed space high dimensional; thus though the classifier is a hyperplane in the high-dimensional feature space, it may be non-linear in the original input space.

If the kernel used is a Gaussian radial basis function, the corresponding feature space is a Hilbert space of infinite dimension. Maximum margin classifiers are well regularized, so the infinite dimension does not spoil the results. Some common kernels include,

Polynomial (homogeneous): k(xᵢ,xⱼ)=(xᵢ·xⱼ)^{d}

Polynomial (inhomogeneous): k(xᵢ,xⱼ)=(xᵢ·xⱼ+1)^{d}

Radial Basis Function: k(xᵢ,xⱼ)=exp(−γ∥xᵢ−xⱼ∥²), for γ>0
Gaussian Radial basis function:

$$k ( {x}_{i} , {x}_{j} ) = exp ( - \frac{{‖ {x}_{i} - {x}_{j} ‖}^{2}}{2 {σ}^{2}} )$$

Hyperbolic tangent: k(xᵢ,xⱼ)=tan h (κxᵢ·xⱼ+c), for some (not every) κ>0 and c<0
   The kernel is related to the transform φ(xᵢ) by the equation k(xᵢ,xⱼ)=φ(xᵢ)·φ(xⱼ). The value w is also in the transformed space, with w=Σᵢαᵢcᵢφ(xᵢ)·Dot products with w for classification can again be computed by the kernel trick, i.e. w·φ(x)=Σᵢαᵢcᵢk(xᵢ, x). However, there does not in general exist a value w′ such that w·φ(x)=k(w′, x).
   After the w and b are determined employing the above mentioned methods these can be used for classifying new datasets (e.g. a expression profile of a patient with p miRNA biomarkers and corresponding xᵢ intensity values).

As mentioned above, the method of the present invention is for diagnosing a lung disease (e.g. sarcoidosis, lung cancer and/or COPD) in a patient (e.g. human or animal). Preferably, the diagnosis comprises (i) determining the occurrence/presence of a lung disease (e.g. sarcoidosis, lung cancer and/or COPD), (ii) monitoring the course of a lung disease (e.g. sarcoidosis, lung cancer and/or COPD), (iii) staging of a lung disease (e.g. sarcoidosis, lung cancer and/or COPD), (iv) measuring the response of a patient with a lung disease (e.g. sarcoidosis, lung cancer and/or COPD) to therapeutic intervention, and/or (v) segmentation of a patient suffering from a lung disease (e.g. sarcoidosis, lung cancer and/or COPD). Further, the method of the present invention is for prognosis of a lung disease (e.g. sarcoidosis, lung cancer and/or COPD) in a patient (e.g. human or animal). Preferably, the prognosis comprises (i) identifying of a patient who has a risk to develop a lung disease (e.g. sarcoidosis, lung cancer and/or COPD), (ii) predicting/estimating the occurrence, preferably the severity of occurrence, of a lung disease (e.g. sarcoidosis, lung cancer and/or COPD), and/or (iii) predicting the response of a patient with a lung disease (e.g. sarcoidosis, lung cancer and/or COPD) to therapeutic intervention.

Like highlighted above, the reference miRNA expression profiles may be classified using machine learning approaches in order to compute accuracy, specificity, and sensitivity for the diagnosis and/or prognosis of a lung disease (e.g. sarcoidosis, lung cancer and/or COPD) (see experimental section for more details). Examples of miRNA sets (signatures) that performed best for the diagnosis of a lung disease (e.g. sarcoidosis, lung cancer and/or COPD) according to their accuracy, specificity, and sensitivity are sets of miRNAs shown in FIG. 15, FIG. 16 and FIG. 17. Further examples of miRNAs sets (signatures) that performed best for the diagnosis of a lung disease (e.g. sarcoidosis, lung cancer and/or COPD) according to their accuracy, specificity, and sensitivity are sets of miRNAs shown in FIGS. 4 to 12.

Preferably, the reference (e.g. (average) reference expression profile (data)) and/or the algorithm or mathematical function is (are) stored in a database, e.g. an internet database, a centralized, or a decentralized database. It is also preferred that the reference (e.g. (average) reference expression profile (data)) and/or the algorithm or mathematical function is (are) stored on a data carrier, e.g. electronically data carrier. It is further preferred that the reference (e.g. (average) reference expression profile (data)) and/or the algorithm or mathematical function is (are) comprised in a computer program stored on an electronically data carrier.

In a third aspect, the present invention provides means for diagnosing and/or prognosing of a lung disease, preferably sarcoidosis, lung cancer, and/or chronic obstructive pulmonary disease (COPD), comprising one or more polynucleotide(s) (probe(s)) according to the first aspect of the present invention. It is particularly preferred that the above mentioned means are (i) for differential diagnosis of sarcoidosis with respect to lung cancer and/or COPD, (ii) for differential diagnosis of lung cancer with respect to sarcoidosis and/or COPD, or (iii) for differential diagnosis of COPD with respect to sarcoidosis and/or lung cancer and comprise one or more polynucleotide(s) (probe(s)) according to the first aspect of the present invention (see above).

More preferably, the means for diagnosing and/or prognosing of a lung disease, preferably sarcoidosis, lung cancer, and/or chronic obstructive pulmonary disease (COPD), comprise, essentially consist of, or consist of a solid support, substrate, surface, platform or matrix comprising one or more polynucleotide(s) (probe(s)) according to the first aspect of the present invention. It is particularly preferred that the above mentioned means are (i) for differential diagnosis of sarcoidosis with respect to lung cancer and/or COPD, (ii) for differential diagnosis of lung cancer with respect to sarcoidosis and/or COPD, or (iii) for differential diagnosis of COPD with respect to sarcoidosis and/or lung cancer and comprise one or more polynucleotide(s) (probe(s)) according to the first aspect of the present invention.

Most preferably, the means for diagnosing and/or prognosing of a lung disease, preferably sarcoidosis, lung cancer, and/or chronic obstructive pulmonary disease (COPD), comprise, essentially consist of, or consist of a microarray/biochip comprising one or more polynucleotide(s) (probe(s)) according to the first aspect of the present invention. It is particularly preferred that the above mentioned means are (i) for differential diagnosis of sarcoidosis with respect to lung cancer and/or COPD, (ii) for differential diagnosis of lung cancer with respect to sarcoidosis and/or COPD, or (iii) for differential diagnosis of COPD with respect to sarcoidosis and/or lung cancer and comprise one or more polynucleotide(s) (probe(s)) according to the first aspect of the present invention.

The polynucleotide(s) (probe(s)) may also be comprised as polynucleotide fragments, polynucleotide variants, or polynucleotide fragment variants in the means for diagnosing and/or prognosing of a lung disease, preferably sarcoidosis, lung cancer, and/or chronic obstructive pulmonary disease (COPD). For example, polynucleotide fragments, polynucleotide variants, or polynucleotide fragment variants may be comprised in the solid support, substrate, surface, platform or matrix, preferably microarray/biochip. Said polynucleotide fragments, polynucleotide variants, or polynucleotide fragment variants may be attached or linked to the solid support, substrate, surface, platform or matrix, preferably microarray/biochip. As to the definition of said polynucleotide fragments, polynucleotide variants, or polynucleotide fragment variants and as to the preferred polynucleotide (probe) or sets of polynucleotides (probes), it is referred to the first aspect of the present invention.

The terms “biochip” or “microarray”, as used herein, refer to a solid phase comprising an attached or immobilized polynucleotide described herein as probe or a set (plurality) of polynucleotides described herein attached or immobilized as probes. The polynucleotide probes may be capable of hybridizing to a target sequence, such as a complementary miRNA or miRNA* sequence, under stringent hybridization conditions. The polynucleotide probes may be attached or immobilized at spatially defined locations on the solid phase. One or more than one nucleotide (probe) per target sequence may be used. The polynucleotide probes may either be synthesized first, with subsequent attachment to the biochip, or may be directly synthesized on the biochip. The solid phase may be a material that may be modified to contain discrete individual sites appropriate for the attachment or association of the polynucleotide probes and is amenable to at least one detection method. Representative examples of solid phase materials include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. The solid phase may allow optical detection without appreciably fluorescing. The solid phase may be planar, although other configurations of solid phase may be used as well. For example, polynucleotide probes may be placed on the inside surface of a tube, for flow-through sample analysis to minimize sample volume. Similarly, the solid phase may be flexible, such as flexible foam, including closed cell foams made of particular plastics. The solid phase of the biochip and the probe may be modified with chemical functional groups for subsequent attachment of the two. For example, the biochip may be modified with a chemical functional group including, but not limited to, amino groups, carboxyl groups, oxo groups or thiol groups. Using these functional groups, the probes may be attached using functional groups on the probes either directly or indirectly using a linker. The polynucleotide probes may be attached to the solid support by either the 5′ terminus, 3′ terminus, or via an internal nucleotide. The polynucleotide probe may also be attached to the solid support non-covalently. For example, biotinylated polynucleotides can be made, which may bind to surfaces covalently coated with streptavidin, resulting in attachment. Alternatively, polynucleotide probes may be synthesized on the surface using techniques such as photopolymerization and photolithography. In the context of the present invention, the terms “biochip” and “microarray” are interchangeable used.

The terms “attached” or “immobilized”, as used herein, refer to the binding between the polynucleotide and the solid support/phase and may mean that the binding between the polynucleotide probe and the solid support is sufficient to be stable under conditions of binding, washing, analysis and removal. The binding may be covalent or non-covalent. Covalent bonds may be formed directly between the polynucleotide and the solid support or may be formed by a cross linker or by inclusion of specific reactive groups on either the solid support or the polynucleotide, or both. Non-covalent binding may be electrostatic, hydrophilic and hydrophobic interactions or combinations thereof. Immobilization or attachment may also involve a combination of covalent and non-covalent interactions.

More preferably, said means for diagnosing and/or prognosing of a lung disease, preferably sarcoidosis, lung cancer, and/or chronic obstructive pulmonary disease (COPD) comprise, essentially consist of, or consist of a set of beads or a set of microspheres comprising one or more polynucleotide(s) (probe(s)) according to the first aspect of the present invention. It is particularly preferred that the above mentioned means are (i) for differential diagnosis of sarcoidosis with respect to lung cancer and/or COPD, (ii) for differential diagnosis of lung cancer with respect to sarcoidosis and/or COPD, or (iii) for differential diagnosis of COPD with respect to sarcoidosis and/or lung cancer and comprise one or more polynucleotide(s) (probe(s)) according to the first aspect of the present invention.

Preferably, the above mentioned polynucleotide(s) is (are) attached to or immobilized on the beads or microspheres, e.g. via a covalent or non-covalent linkage (see above). Preferably, said beads or microspheres are made of a synthetic material, e.g. polystyrene, polyethylene or polypropylene. It is preferred that said beads or microspheres have a mean diameter of between 2 to 20 microns, preferably 4 to 10 microns, most preferably 5 to 7 microns, i.e. 2, 3, 4, 5, 6, 7, 8, 9, 10, 11, 12, 13, 14, 15, 16, 17, 18, 19, or 20 microns. It is also preferred that said beads or microspheres are internally dyed, preferably with red and infrared fluorophores. For example, the bead or microsphere setting from Luminex may be used.

In a fourth aspect, the present invention provides a kit for diagnosing and/or prognosing a lung disease, preferably sarcoidosis, lung cancer, and/or chronic obstructive pulmonary disease (COPD), comprising
(i) means for determining an expression profile of one or more miRNA(s) in a biological sample from a patient, wherein the nucleotide sequence of said miRNA or the nucleotide sequences of said miRNAs is (are) selected from the group consisting of SEQ ID NOs: 1 to 391, a fragment thereof, and a sequence having at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity thereto, and
(ii) optionally at least one reference and/or algorithm or mathematical function comprised on at least one data carrier.

Preferably, means for determining an expression profile of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 miRNAs in a biological sample (e.g. body fluid sample such as blood or urine sample or tissue sample) from a patient (e.g. human or animal) are comprised in the kit of the present invention, wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

Said means may be one or more polynucleotide(s) (probe(s)) according to the first aspect of the present invention (see above); means according to the third aspect of the present invention (see above); primer suitable to perform reverse transcriptase reaction and/or real time polymerase chain reaction such as quantitative polymerase chain reaction; and/or means for conducting next generation sequencing.

It is preferred that said kit comprises means according to the third aspect of the present invention (see above). Thus, said kit preferably comprises means for diagnosing and/or prognosis of a lung disease, preferably sarcoidosis, lung cancer, and/or chronic obstructive pulmonary disease (COPD), comprising one or more polynucleotide(s) (probe(s)) according to the first aspect of the present invention, or more preferably comprises means for diagnosing and/or prognosis of a a lung disease, preferably sarcoidosis, lung cancer, and/or chronic obstructive pulmonary disease (COPD), wherein said means comprise a microarray/biochip, a set of beads, or a set of microspheres comprising one or more polynucleotide(s) (probe(s)) according to the first aspect of the present invention.

It is particularly preferred that said kit comprises
(ia) one or more polynucleotide(s) (probe(s)) according to the first aspect of the present invention, and
(ib) optionally means to extract total RNA (or fractions thereof, e.g. miRNA) from a biological sample, e.g. blood or tissue sample, and/or means to immobilize the polynucleotide(s) onto a solid support or matrix, e.g. biochip,
   for determining an expression profile of one or more miRNA(s) in a biological sample (e.g. blood or tissue sample) from a patient (e.g. human or animal), wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

It is more preferred that said kit comprises
(ia) a solid support, substrate, surface, platform or matrix comprising one or more polynucleotide(s) (probe(s)) according of the first aspect of the present invention, and
(ib) optionally at least one of the means selected from the group consisting of: means to extract total RNA (or fractions thereof, e.g. miRNA) from a biological sample (e.g. blood or tissue sample), means for input/injection of a biological sample (e.g. blood sample), means for holding the solid support, substrate, platform or matrix comprising the polynucleotide(s) (probe(s)), means for labelling the isolated miRNA (e.g. NTP/biotin-NTP), means to carry out hybridization, means to carry out enzymatic reactions (e.g. exonuclease I and/or Klenow enzyme), means for washing steps, means for detecting the hybridization signal, and means for analysing the detected hybridization signal,
   for determining an expression profile of one or more miRNA(s) in a biological sample (e.g. blood or tissue sample) from a patient (e.g. human or animal), wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

Preferably, the above mentioned polynucleotide(s) is (are) attached to or immobilized on the solid support, substrate, surface, platform or matrix.

It is most preferred that said kit comprises
(ia) a microarray/biochip comprising one or more polynucleotide(s) (probe(s)) according of the first aspect of the present invention, and
(ib) optionally at least one of the means selected from the group consisting of: means to extract total RNA (or fractions thereof, e.g. miRNA) from a biological sample (e.g. blood or tissue sample), means for input/injection of a biological sample (e.g. blood sample), means for holding the microarray/biochip comprising the polynucleotide(s) (probe(s)), means for labelling the isolated miRNA (e.g. NTP/biotin-NTP), means to carry out hybridization, means to carry out enzymatic reactions (e.g. exonuclease I and/or Klenow enzyme), means for washing steps, means for detecting the hybridization signal, and means for analysing the detected hybridization signal,
   for determining an expression profile of one or more miRNA(s) in a biological sample (e.g. blood or tissue sample) from a patient (e.g. human or animal), wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

Preferably, the above mentioned polynucleotide(s) is (are) attached to or immobilized on a microarray/biochip.

It is also most preferred that said kit comprises
(ia) a set of beads or a set of microspheres comprising one or more polynucleotide(s) (probe(s)) according of the first aspect of the present invention, and
(ib) optionally at least one of the means selected from the group consisting of: means to extract total RNA (or fractions thereof, e.g. miRNA) from a biological sample (e.g. blood or tissue sample), means for input/injection of a biological sample (e.g. blood sample), means for labelling the isolated miRNA (e.g. NTP/biotin-NTP), means to carry out hybridization, means to carry out enzymatic reactions (e.g. exonuclease I and/or Klenow enzyme), means for washing steps, means for detecting the hybridization signal, and means for analysing the detected hybridization signal,
   for determining an expression profile of one or more miRNA(s) in a biological sample (e.g. blood or tissue sample) from a patient (e.g. human or animal), wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto.

Preferably, the above mentioned polynucleotide(s) is (are) attached to or immobilized on the beads or microspheres.

It is also preferred that said kit comprises means for conducting next generation sequencing in order to determine an expression profile of one or more miRNA(s) in a biological sample (e.g. blood or tissue sample) from a patient (e.g. human or animal), wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto. Said kit optionally comprises means selected from the group consisting of: means to collect a biological sample (e.g. blood or tissue sample) from a patient (e.g. a human or an animal), means for conserving the RNA-fraction within the biological sample (e.g. blood or tissue sample), and means to extract total RNA (or fractions thereof, e.g. miRNA) from a biological sample (e.g. blood or tissue sample).

It is further preferred that said kit comprises means (e.g. a setting or settings) for conducting a real time polymerase chain reaction (RT-PCR), preferably a real time quantitative polymerase chain reaction (RT-qPCR), in order to determine an expression profile of one or more miRNA(s) in a biological sample (e.g. blood or tissue sample) from a patient (e.g. a human or an animal), wherein the nucleotide sequence of said miRNA or the nucleotide sequences of said miRNAs is (are) selected from the group consisting of SEQ ID NOs: 1 to 391, a fragment thereof, and a sequence having at least 80% sequence identity thereto. The setting for conduction a RT-PCR reaction, preferably a RT-qPCR reaction, may be combined with a setting for conducting a reverse transcription reaction, or may be two separate settings, meaning that both reactions, namely the reverse transcription reaction and RT-PCR reaction, preferably the RT-qPCR reaction, can be run together in one setting or separately in two settings.

As to the definition of the miRNA fragment, miRNA variant, or miRNA fragment variant mentioned above and as to the preferred miRNA(s) determined by the means of (i), it is referred to the first and second aspect of the present invention.

Preferably, the nucleotide sequence of said miRNA or the nucleotide sequences of said miRNAs determined in (i) is (are) selected from the group consisting of SEQ ID NOs: 1 to 231 (see lists 1, 2, 4 and 7 of FIG. 18). It is preferred that the kit comprising means for the determination of an expression profile of the above mentioned miRNA(s) is for diagnosing and/or prognosing of sarcoidosis.

In this respect it is particularly preferred that the nucleotide sequence of said miRNA or the nucleotide sequences of said miRNAs determined in (i) is (are) selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (see list 1 of FIG. 18). It is further particularly preferred that the nucleotide sequences of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 miRNAs determined in (i) are selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (see list 1 of FIG. 18). Preferably, the kit comprising means for the determination of an expression profile of the above mentioned miRNA(s) is for diagnosing and/or prognosing of sarcoidosis.

More preferably, the nucleotide sequence of said miRNA or the nucleotide sequences of said miRNAs determined in (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18). It is preferred that the kit comprising means for the determination of an expression profile of the above mentioned miRNA(s) is for differential diagnosis (of sarcoidosis) with respect to lung cancer and/or COPD.

Most preferably, the nucleotide sequence of said miRNA or the nucleotide sequences of said miRNAs determined in (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (see list 2 of FIG. 18). It is preferred that the kit comprising means for the determination of an expression profile of the above mentioned miRNA(s) is for differential diagnosis (of sarcoidosis) with respect to lung cancer. Further, most preferably, the nucleotide sequence of said miRNA or the nucleotide sequences of said miRNAs determined in (i) is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18). It is preferred that the kit comprising means for the determination of an expression profile of the above mentioned miRNA(s) is for differential diagnosis (of sarcoidosis) with respect to lung cancer and COPD. Furthermore, most preferably, the nucleotide sequence of said miRNA or the nucleotide sequences of said miRNAs determined in (i) is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18). It is preferred that the kit comprising means for the determination of an expression profile of the above mentioned miRNA(s) is for differential diagnosis (of sarcoidosis) with respect to COPD.

In preferred embodiments, the nucleotide sequences of said miRNAs are the nucleotide sequences according to Set No. 1 to 42 (i.e. SH-1 to SH-42) of FIG. 15.

Preferably, the nucleotide sequence of said miRNA or the nucleotide sequences of said miRNAs determined in (i) is (are) selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, 329 (see list 3 of FIG. 18), 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). It is preferred that the kit comprising means for the determination of an expression profile of the above mentioned miRNA(s) is for diagnosing and/or prognosing of lung cancer.

In this respect it is particularly preferred that the nucleotide sequence of said miRNA or the nucleotide sequences of said miRNAs determined in (i) is (are) selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, and 329 (see list 3 of FIG. 18). It is further particularly preferred that the nucleotide sequences of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 miRNAs determined in (i) are selected from the group consisting of SEQ ID NOs: 233, 234, 235, 237, 240, 241, 242, 243, 245, 246, 248, 249, 251, 252, 254, 255, 258, 259, 261, 262, 263, 266, 268, 269, 270, 272, 273, 275, 276, 277, 278, 279, 282, 283, 284, 285, 288, 289, 290, 291, 292, 293, 294, 297, 298, 299, 300, 301, 303, 305, 308, 310, 312, 314, 317, 321, 322, 325, 326, and 329 (see list 3 of FIG. 18). Preferably, the kit comprising means for the determination of an expression profile of the above mentioned miRNA(s) is for diagnosing and/or prognosing of lung cancer.

More preferably, the nucleotide sequence of said miRNA or the nucleotide sequences of said miRNAs determined in (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (see list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). It is preferred that the kit comprising means for the determination of an expression profile of the above mentioned miRNA(s) is for differential diagnosis (of lung cancer) with respect to sarcoidosis and/or COPD.

Most preferably, the nucleotide sequence of said miRNA or the nucleotide sequences of said miRNAs determined in (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (see list 2 of FIG. 18). It is preferred that the kit comprising means for the determination of an expression profile of the above mentioned miRNA(s) is for differential diagnosis (of lung cancer) with respect to sarcoidosis. Further, most preferably, the nucleotide sequence of said miRNA or the nucleotide sequences of said miRNAs determined in (i) is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18). It is preferred that the kit comprising means for the determination of an expression profile of the above mentioned miRNA(s) is for differential diagnosis (of lung cancer) with respect to sarcoidosis and COPD. Furthermore, most preferably, the nucleotide sequence of said miRNA or the nucleotide sequences of said miRNAs determined in (i) is (are) selected from the group consisting of SEQ ID NOs: 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). It is preferred that the kit comprising means for the determination of an expression profile of the above mentioned miRNA(s) is for differential diagnosis (of lung cancer) with respect to COPD.

In preferred embodiments, the nucleotide sequences of said miRNAs are the nucleotide sequences according to Set No. 1 to 42 of FIG. 16.

Preferably, the nucleotide sequence of said miRNA or the nucleotide sequences of said miRNAs determined in (i) is (are) selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391 (see list 6 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, 231 (see list 7 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). It is preferred that the kit comprising means for the determination of an expression profile of the above mentioned miRNA(s) is for diagnosing and/or prognosing of COPD.

In this respect it is particularly preferred that the nucleotide sequence of said miRNA or the nucleotide sequences of said miRNAs determined in (i) is (are) selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, and 391 (see list 6 of FIG. 18). It is further particularly preferred that the nucleotide sequences of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 miRNAs determined in (i) are selected from the group consisting of SEQ ID NOs: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, and 391 (see list 6 of FIG. 18). Preferably, the kit comprising means for the determination of an expression profile of the above mentioned miRNA(s) is for diagnosing and/or prognosing of COPD.

More preferably, the nucleotide sequence of said miRNA or the nucleotide sequences of said miRNAs determined in (i) is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, 231 (see list 7 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (see list 4 of FIG. 18), 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). It is preferred that the kit comprising means for the determination of an expression profile of the above mentioned miRNA(s) is for differential diagnosis (of COPD) with respect to sarcoidosis and/or lung cancer.

Most preferably, the nucleotide sequence of said miRNA or the nucleotide sequences of said miRNAs determined in (i) is (are) selected from SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (see list 7 of FIG. 18). It is preferred that the kit comprising means for the determination of an expression profile of the above mentioned miRNA(s) is for differential diagnosis (of COPD) with respect to sarcoidosis. Further, most preferably, the nucleotide sequence of said miRNA or the nucleotide sequences of said miRNAs determined in (i) is (are) selected from SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (see list 4 of FIG. 18). It is preferred that the kit comprising means for the determination of an expression profile of the above mentioned miRNA(s) is for differential diagnosis (of COPD) with respect to sarcoidosis and lung cancer.

Furthermore, most preferably, the nucleotide sequence of said miRNA or the nucleotide sequences of said miRNAs determined in (i) is (are) selected from SEQ ID NOs: 232, 236, 238, 239, 244, 247, 250, 253, 256, 257, 260, 264, 265, 267, 271, 274, 280, 281, 286, 287, 295, 296, 302, 304, 306, 307, 309, 311, 313, 315, 316, 318, 319, 320, 323, 324, 327, and 328 (see list 5 of FIG. 18). It is preferred that the kit comprising means for the determination of an expression profile of the above mentioned miRNA(s) is for differential diagnosis (of COPD) with respect to lung cancer.

In preferred embodiments, the nucleotide sequences of said miRNAs are the nucleotide sequences according to Set No. 1 to 42 of FIG. 17. As mentioned above, the kit of the present invention may further optionally comprise at least one reference and/or algorithm or mathematical function comprised on at least one data carrier (see item (ii)).

The at least one reference may be any reference which allows for the diagnosis and/or prognosis of a lung disease such as sarcoidosis, lung cancer and/or COPD, e.g. an indicated value or values, and/or the at least one algorithm or mathematical function may be any algorithm or mathematical function which allows for the diagnosis and/or prognosis of a lung disease such as sarcoidosis, lung cancer and/or COPD.

It is preferred that said reference is a reference expression profile (data) of at least one subject, preferably the reference is an average expression profile (data) of at least 2 to 40 subjects, more preferably at least 10 to 25 subjects, and most preferably at least 15 to 20 subjects, with a (at least one) known clinical condition, wherein the reference expression profile is the profile of a (one) miRNA that has a nucleotide sequence that essentially corresponds (is essentially identical), preferably that corresponds (is identical), to the nucleotide sequence of the miRNA which expression profile is determinable by the means of (i) or is the profile of (more than one) miRNAs that have nucleotide sequences that essentially correspond (are essentially identical), preferably that correspond (are identical), to the nucleotide sequences of the miRNAs which expression profile is determinable by the means of (i). More preferably, the known clinical condition is healthiness or a lung disease (e.g. sarcoidosis, lung cancer or COPD). Most preferably, the known clinical condition is selected from the group consisting of healthiness, sarcoidosis, lung cancer, and COPD.

The term “essentially corresponds (essentially identical)”, as used in the context (of the fourth aspect) of the present invention, means that the miRNA(s) which expression profile is determinable by the means of (i) and the miRNA(s) of the reference expression profile may slightly differ in their nucleotide sequence, whereas said difference is so marginal that it may still allow for the diagnosis and/or prognosis of a lung disease such as sarcoidosis, lung cancer and/or COPD. Preferably, the nucleotide sequence(s) of the miRNA(s) which expression profile is determinable by the means of (i) and the nucleotide sequence(s) of the miRNA(s) of the reference expression profile differ in 1 to 5, more preferably in 1 to 3, and most preferably in 1 to 2 nucleotides, i.e. in 1, 2, 3, 4, or 5 nucleotides. It is preferred that said difference resides in 1 to 5, more preferably 1 to 3, and most preferably 1 to 2 nucleotide mutations, i.e. 1, 2, 3, 4, or 5 nucleotide mutations (e.g. substitutions, additions, insertions, and/or deletions).

Preferably, it is indicated in which type of biological sample (e.g. tissue sample or body fluid sample such as blood and/or urine sample) and/or from which (control) subject(s) (e.g. species, gender and/or age or stage of life), the (average) reference expression profile (data), which is (are) provided with the kit, has (have) been determined. It is preferred that the expression profile (data) of the patient will be determined in the same type of biological sample (e.g. tissue sample or body fluid sample such as blood and/or urine sample) and/or will be obtained from a patient of the same species, gender and/or similar age or stage of life.

Preferably, the algorithm or mathematical function is obtained from a reference expression profile of at least one subject, preferably of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with a (at least one) known clinical condition, wherein the reference expression profile is the profile of a (one) miRNA that has a nucleotide sequence that essentially corresponds (is essentially identical), preferably that corresponds (is identical), to the nucleotide sequence of the miRNA which expression profile is determinable by the means of (i), or is the profile of (more than one) miRNAs that have nucleotide sequences that essentially correspond (are essentially identical), preferably that correspond (are identical), to the nucleotide sequences of the miRNAs which expression profile is determinable by the means of (i). More preferably, the known clinical condition is healthiness or a lung disease (e.g. sarcoidosis, lung cancer or COPD). Most preferably, the known clinical condition is selected from the group consisting of healthiness, sarcoidosis, lung cancer, and COPD.

It is particularly preferred that the algorithm or mathematical function is obtained from a reference expression profile of at least one subject, preferably of at least 2 to 40 subjects, more preferably of at least 10 to 25 subjects, and most preferably of at least 15 to 20 subjects, i.e. of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with at least one known clinical condition, wherein the reference expression profile is the profile of a miRNA that has a nucleotide sequence that essentially corresponds (is essentially identical), preferably that corresponds (is identical), to the nucleotide sequence of the miRNA which expression profile is determinable by the means of (i) or is the profile of miRNAs that have nucleotide sequences that essentially correspond (are essentially identical), preferably that correspond (are identical), to the nucleotide sequences of the miRNAs which expression profile is determinable by the means of (i). More preferably, the at least one known clinical condition is healthiness or a lung disease (e.g. sarcoidosis, lung cancer or COPD). Most preferably, the at least one known clinical condition is selected from the group consisting of healthiness, sarcoidosis, lung cancer, and COPD.

It is also particularly preferred that the algorithm or mathematical function is obtained from reference expression profiles of at least two subjects, preferably of at least 3 to 40 subjects, more preferably of at least 10 to 25 subjects, and most preferably of at least 15 to 20 subjects, i.e. of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subjects, with at least two known clinical conditions, preferably 2 to 4 (e.g. 2, 3, or 4) known clinical conditions, wherein the reference expression profiles are the profiles of a miRNA that has a nucleotide sequence that essentially corresponds (is essentially identical), preferably that corresponds (is identical) to the nucleotide sequence of the miRNA which expression profile is determinable by the means of (i), or are the profiles of miRNAs that have nucleotide sequences that essentially correspond (are essentially identical), preferably that correspond (are identical), to the nucleotide sequences of the miRNAs which expression profile is determinable by the means of (i). Preferably, the at least two known clinical conditions are selected from the group consisting of healthiness, sarcoidosis, lung cancer, and COPD.

More preferably, the algorithm or mathematical function is obtained from (a) reference expression profile(s) of
(i) at least one subject with the known clinical condition of sarcoidosis,
(ii) at least one subject with the known clinical condition of healthiness,
(iii) at least one subject with the known clinical condition of lung cancer, and/or
(iv) at least one subject with the known clinical condition of COPD,
   wherein the reference expression profile(s) is (are) the profile(s) of a miRNA that has a nucleotide sequence that essentially corresponds (is essentially identical), preferably that corresponds (is identical), to the nucleotide sequence of the miRNA which expression profile is determinable by the means of (i), or is (are) the profile(s) of miRNAs that have nucleotide sequences that essentially correspond (are essentially identical), preferably that correspond (are identical), to the nucleotide sequences of the miRNAs which expression profile is determinable by the means of (i).

It is further preferred that the algorithm or mathematical function is obtained using a machine learning approach. The inventors of the present invention surprisingly found that the application of a machine learning approach (e.g. t-test, AUC, support vector machine, hierarchical clustering, or k-means) leads to the obtainment of an algorithm or a mathematical function that is trained by the reference expression profile(s) (data) mentioned above (see also second aspect of the present invention).

Said data carrier may be a graphically data carrier such as an information leaflet or an information sheet (e.g. for comparing tested single reference miRNA biomarkers with the expression profile data of a patient to be diagnosed and/or prognosed) or an electronically data carrier such as a floppy disk, a compact disk (CD), or a digital versatile disk (DVD) (e.g. for comparing tested sets of miRNA biomarkers with the expression profile data of a patient to be diagnosed and/or prognosed). Said reference, preferably said (average) reference expression profile (data), and/or said algorithm or mathematical function may further be comprised in a computer program which is saved on an electronically data carrier.

The kit may alternatively comprise an access code which allows the access to a database, e.g. an internet database, a centralized, or a decentralized database, where (i) the reference, preferably the (average) reference expression profile (data), and/or the algorithm or mathematical function is (are) comprised, or (ii) where a computer program comprising the reference, preferably the (average) reference expression profile (data), and/or the algorithm or mathematical function can be downloaded.

More than one reference and/or more than one algorithm or mathematical function, e.g. 2, 3, 4, 5, or more references and/or algorithms or mathematical functions, may be comprised on one or more data carrier. For example, the kit may comprise (i) references (data), preferably (average) reference expression profile(s) (data), which may be comprised on an information leaflet and/or on a compact disk (CD), e.g. two expression profiles (data), for example, one from a subject(s) known to be healthy and one from a subject(s) known to have a lung disease such as sarcoidosis, lung cancer or COPD, and/or (ii) algorithms or mathematical functions, which may be comprised on a compact disc (CD) and/or on a digital versatile disk (DVD), e.g. two algorithms or mathematical functions, for example, one obtained from a reference expression profile of a subject(s) known to be healthy and one obtained from a reference expression profile of a subject(s) known to have a lung disease such as sarcoidosis, lung cancer or COPD.

Said two or more references (e.g. reference expression profiles) and/or algorithms or mathematical functions may be accessible by on or more access codes as mentioned above which may alternatively be comprised in the kit.

As to the reference or preferred embodiments of the reference (e.g. reference expression profile(s) (data)) and as to the algorithm or mathematical function, it is also referred to the second aspect of the present invention (see above).

### BRIEF DESCRIPTION OF THE DRAWINGS

FIG. 1: MiRNAs for diagnosing and/or prognosing of sarcoidosis. Experimental data obtained from analysis of miRNAs according to SEQ ID NOs: 1 to 231 differentially regulated between subjects being healthy (controls) (g1) and subjects having sarcoidosis (g2). Experimental details: SEQ ID NO: sequence identification number, miRNA: identifier of the miRNA according to miRBase, median g1: median intensity obtained from microarray analysis for healthy controls, median g2: median intensity obtained from microarray analysis for subjects having sarcoidosis, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment. The inventors of the present invention found that these miRNAs allow the diagnosis and/or prognosis of sarcoidosis in a biological sample from a patient.

FIG. 2: Listing of miRNAs for diagnosing and/or prognosing of a lung disease such as sarcoidosis, lung cancer, and/or chronic obstructive pulmonary disease (COPD). Said list of miRNAs includes sequences according to SEQ ID NOs: 1 to 391 found to be relevant for diagnosis and/or prognosis of a lung disease such as sarcoidosis, lung cancer, and/or COPD. The miRNAs sequences are based on miRBase version 12.0 to 14.0.

FIG. 3: Classification plot: separating healthy controls (“0”) from individuals suffering from sarcoidosis (“1”).

FIG. 4: Diagnostic miRNA-signature employing SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10; with V1=accuracy (97.7%), V2=specificity (95.6%), V3=sensitivity (100%).

FIG. 5: Diagnostic miRNA-signature employing SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14; with V1=accuracy (97.8%), V2=specificity (95.5%), V3=sensitivity (100%).

FIG. 6: Diagnostic miRNA-signature employing SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19; with V1=accuracy (95.5%), V2=specificity (91.1%), V3=sensitivity (100%).

FIG. 7: Diagnostic miRNA-signature employing SEQ ID NO: 1, SEQ ID NO: 2, SEQ ID NO: 3, SEQ ID NO: 4, SEQ ID NO: 68, SEQ ID NO: 5, SEQ ID NO: 6, SEQ ID NO: 7, SEQ ID NO: 69, SEQ ID NO: 8; with V1=accuracy (95.5%), V2=specificity (93.3%), V3=sensitivity (97.8%).

FIG. 8: Diagnostic miRNA-signature employing SEQ ID NO: 68, SEQ ID NO: 5, SEQ ID NO: 6, SEQ ID NO: 7, SEQ ID NO: 69, SEQ ID NO: 8, SEQ ID NO: 70, SEQ ID NO: 105, SEQ ID NO: 9, SEQ ID NO: 10; with V1=accuracy (95.5%), V2=specificity (95.5%), V3=sensitivity (95.5%).

FIG. 9: Diagnostic miRNA-signature employing SEQ ID NO: 8, SEQ ID NO: 70, SEQ ID NO: 105, SEQ ID NO: 9, SEQ ID NO: 10, SEQ ID NO: 11, SEQ ID NO: 12, SEQ ID NO: 13, SEQ ID NO: 14, SEQ ID NO: 114; with V1=accuracy (95.5%), V2=specificity (91.1%), V3=sensitivity (100%).

FIG. 10: Diagnostic miRNA-signature employing SEQ ID NO: 68, SEQ ID NO: 1, SEQ ID NO: 4, SEQ ID NO: 69, SEQ ID NO: 3, SEQ ID NO: 5, SEQ ID NO: 8, SEQ ID NO: 70, SEQ ID NO: 7, SEQ ID NO: 6; with V1=accuracy (93.3%), V2=specificity (91.0%), V3=sensitivity (95.5%).

FIG. 11: Diagnostic miRNA-signature employing SEQ ID NO: 3, SEQ ID NO: 5, SEQ ID NO: 8, SEQ ID NO: 70, SEQ ID NO: 7, SEQ ID NO: 6, SEQ ID NO: 12, SEQ ID NO: 9, SEQ ID NO: 105, SEQ ID NO: 107; with V1=accuracy (94.4%), V2=specificity (91.1%), V3=sensitivity (97.8%).

FIG. 12: Diagnostic miRNA-signature employing SEQ ID NO: 6, SEQ ID NO: 12, SEQ ID NO: 9, SEQ ID NO: 105, SEQ ID NO: 107, SEQ ID NO: 71, SEQ ID NO: 17, SEQ ID NO: 159, SEQ ID NO: 19, SEQ ID NO: 106; with V1=accuracy (95.5%), V2=specificity (91.0%), V3=sensitivity (100%).

FIG. 13: MiRNAs for diagnosing and/or prognosing of lung cancer. Experimental data obtained from analysis of miRNAs differentially regulated between subjects having lung cancer (g1) and subjects having sarcoidosis (g2). Experimental details: SEQ ID NO: sequence identification number, miRNA: identifier of the miRNA according to miRBase, median g1: median intensity obtained from microarray analysis for subjects having lung cancer, median g2: median intensity obtained from microarray analysis for subjects having sarcoidosis, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment. The inventors of the present invention found that these miRNAs allow the diagnosis and/or prognosis of lung cancer in a biological sample from a patient.

FIG. 14: MiRNAs for diagnosing and/or prognosing of chronic obstructive pulmonary disease (COPD). Experimental data obtained from analysis of miRNAs differentially regulated between subjects having sarcoidosis (g1) and subjects having COPD (g2). Experimental details: SEQ ID NO: sequence identification number, miRNA: identifier of the miRNA according to miRBase, median g1: median intensity obtained from microarray analysis for subjects having sarcoidosis, median g2: median intensity obtained from microarray analysis for subjects having COPD qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment. The inventors of the present invention found that these miRNAs allow the diagnosis and/or prognosis of COPD in a biological sample from a patient.

FIG. 15: Diagnostic sets of miRNAs (miRNA-signatures) for diagnosing and/or prognosing of sarcoidosis and for differential diagnosis with respect to lung cancer and/or COPD. Abbreviations: Set-No.: number of the set of miRNAs (miRNA signature); SEQ ID NO: sequence identification number, miRNA-identifier: identifier of the miRNA according to miRBase; acc=accuracy, spec=specificity, sens=sensitivity.

FIG. 16: Diagnostic sets of miRNAs (miRNA-signatures) for diagnosing and/or prognosing of lung cancer and for differential diagnosis with respect to sarcoidosis and/or COPD. Abbreviations: Set-No.: number of the set of miRNAs (miRNA signature); SEQ ID NO: sequence identification number, miRNA-identifier: identifier of the miRNA according to miRBase; acc=accuracy, spec=specificity, sens=sensitivity.

FIG. 17: Diagnostic sets of miRNAs (miRNA-signatures) for diagnosing and/or prognosing of COPD and for differential diagnosis with respect to lung cancer and/or sarcoidosis. Abbreviations: Set-No.: number of the set of miRNAs (miRNA signature); SEQ ID NO: sequence identification number, miRNA-identifier: identifier of the miRNA according to miRBase; acc=accuracy, spec=specificity, sens=sensitivity.

FIG. 18: It shows in circle diagrams all 391 miRNAs which have been found to be suitable for the diagnosis and/or prognosis of a lung disease (e.g. sarcoidosis, lung cancer, and/or COPD). In detail, circle A consisting of areas 1, 2, 4, and 7 represents the 231 miRNAs found to be differentially regulated in blood samples of subjects having sarcoidosis compared to a cohort of controls (healthy subjects). The inventors of the present invention found that these miRNAs, particularly the 62 miRNAs in area 1, are suitable for the diagnosis and/or prognosis of sarcoidosis in a biological sample from a patient. Further, circle B consisting of areas 3, 2, 4, and 5 represents the 229 miRNAs found to be differentially regulated in blood samples of subjects having sarcoidosis compared to subjects having lung cancer. The inventors of the present invention found that these miRNAs, particularly the 60 miRNAs in area 3, are suitable for the diagnosis and/or prognosis of lung cancer in a biological sample from a patient. Furthermore, circle C consisting of areas 6, 7, 4, and 5 represents the 226 miRNAs found to be differentially regulated in blood samples of subjects having sarcoidosis compared to subjects having COPD. The inventors of the present invention found that these miRNAs, particularly the 62 miRNAs in area 6, are suitable for the diagnosis and/or prognosis of COPD in a biological sample from a patient. In this respect, it should be noted that the detection of at least one miRNA comprised in area 1, at least one miRNA comprised in area 3 and/or at least one miRNA comprised in area 6 may allow the diagnosis and/or prognosis of sarcoidosis, lung cancer, and/or COPD, e.g. the diagnosis and/or prognosis of sarcoidosis, lung cancer, COPD, sarcoidosis and lung cancer, sarcoidosis and COPD, lung cancer and COPD, or sarcoidosis, lung cancer and COPD. In addition, area 2 shared by circles A and B further represents the 43 miRNAs found to be differentially regulated in blood samples of subjects having sarcoidosis compared to a cohort of controls (healthy subjects) and subjects having sarcoidosis compared to subjects having lung cancer; area 7 shared by circles A and C represents the 38 miRNAs found to be differentially regulated in blood samples of subjects having sarcoidosis compared to a cohort of controls (healthy subjects) and subjects having sarcoidosis compared to subjects having COPD; area 5 shared by circles B and C represents the 38 miRNAs found to be differentially regulated in blood samples of subjects having sarcoidosis compared to subjects having lung cancer and subjects having sarcoidosis compared to subjects having COPD; and area 4 shared by circles A, B and C represents the 88 miRNAs found to be differentially regulated in blood samples of subjects having sarcoidosis compared to a cohort of controls (healthy subjects), subjects having sarcoidosis compared to subjects having lung cancer and subjects having sarcoidosis compared to subjects having COPD. The inventors of the present invention also found that these miRNAs that are shared by circles A, B and/or C further allow the differential diagnosis with respect to sarcoidosis, lung cancer, and/or COPD in a biological sample from a patient. This means that (i) the miRNAs that are shared by circles A and B (i.e. miRNAs comprised in area 2) allow to distinguish/differentiate between the lung diseases sarcoidosis and lung cancer, (ii) the miRNAs that are shared by circles A and C (i.e. miRNAs comprised in area 7) allow to distinguish/differentiate between the lung diseases sarcoidosis and COPD, (iii) that the miRNAs that are shared by circles B and C (i.e. miRNAs comprised in area 5) allow to distinguish/differentiate between the lung diseases lung cancer and COPD, and (iv) that the miRNAs that are shared by circles A, B and C (i.e. miRNAs comprised in area 4) allow to distinguish/differentiate between the lung diseases sarcoidosis, lung cancer and COPD.

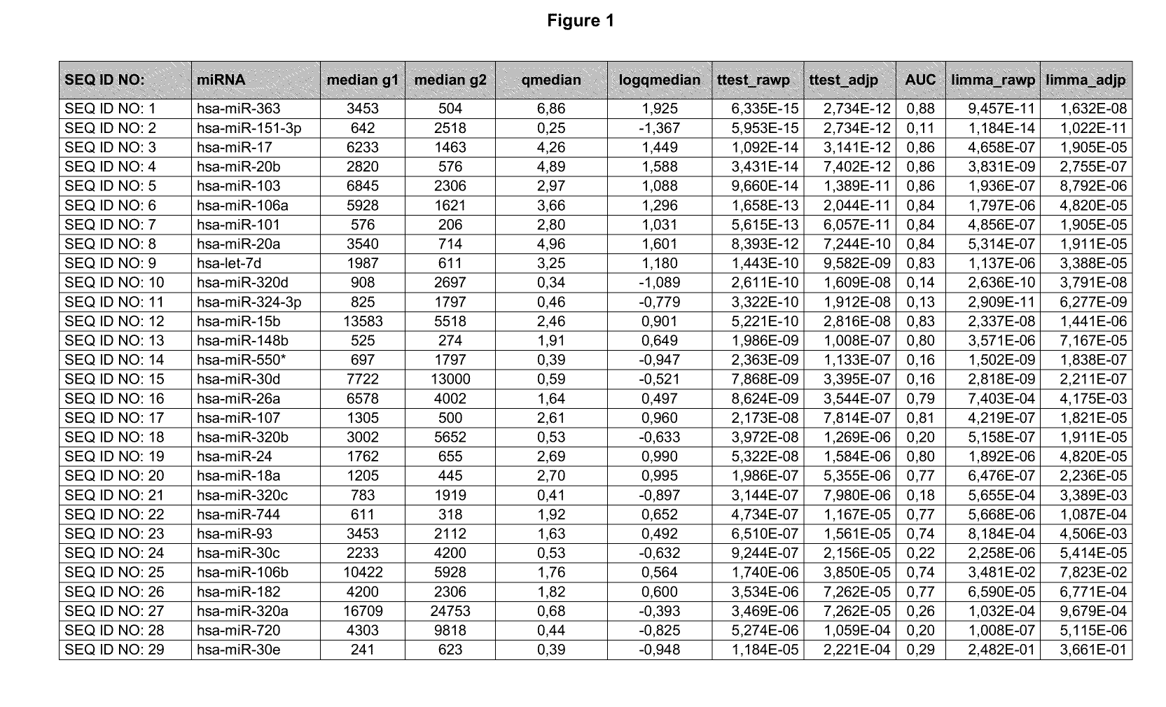

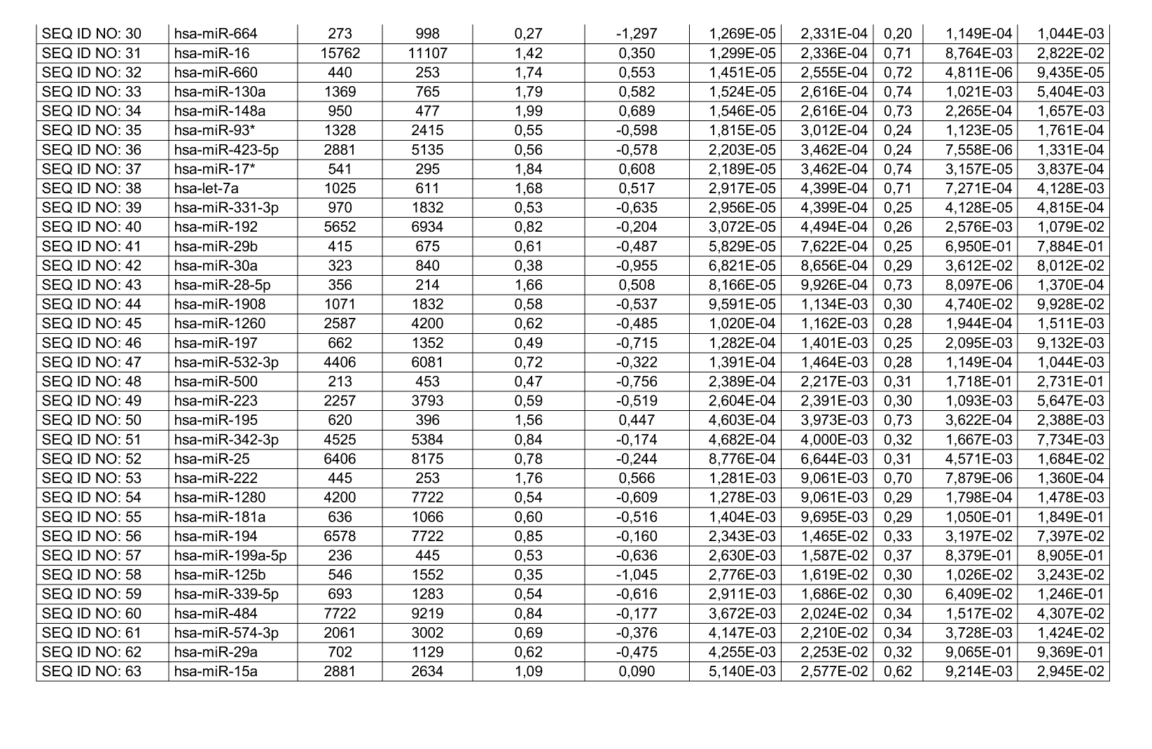

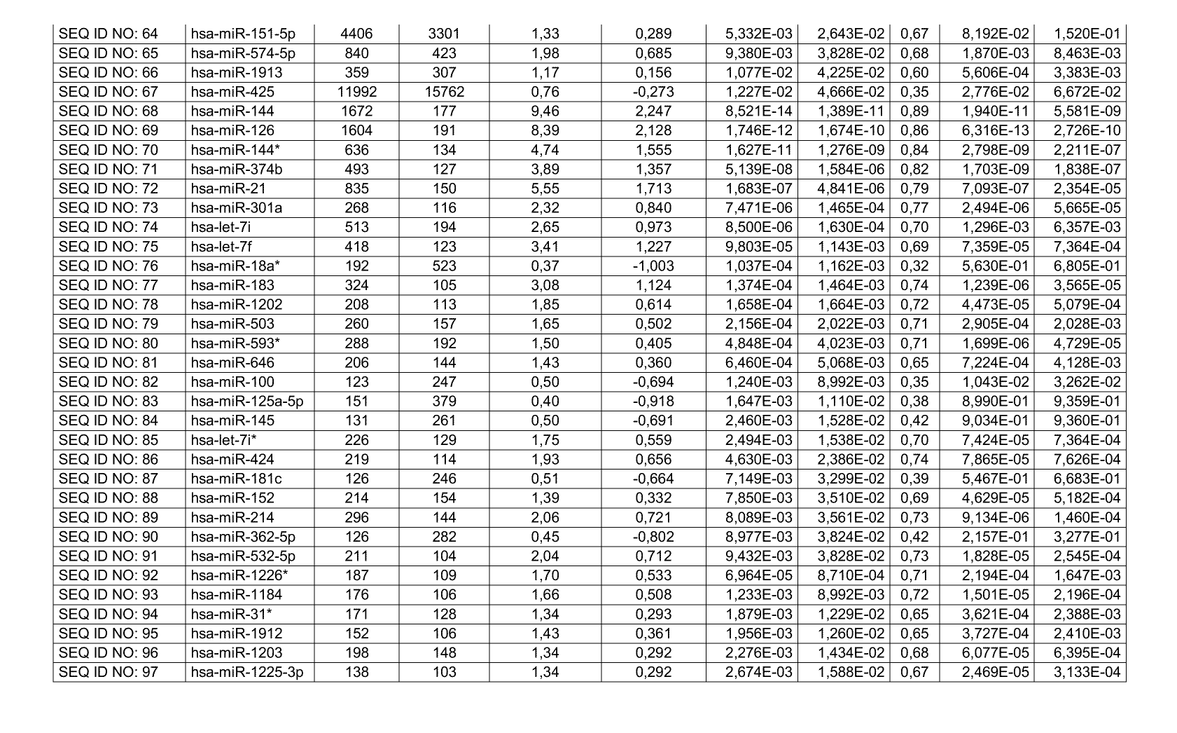

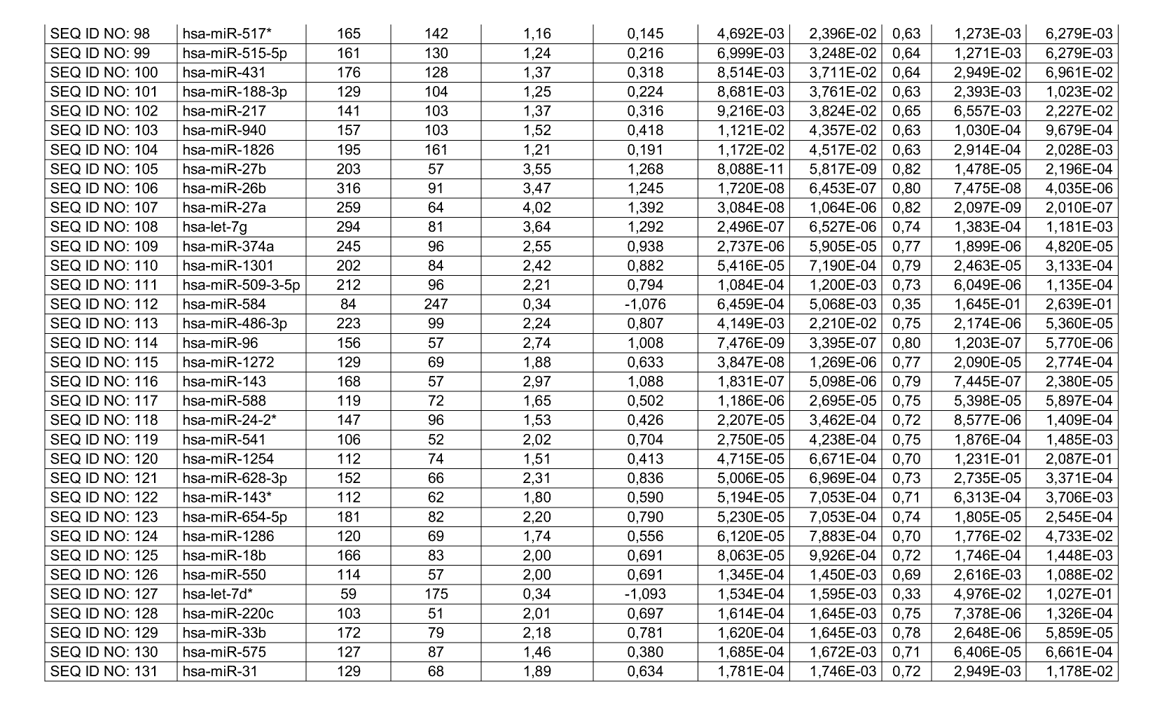

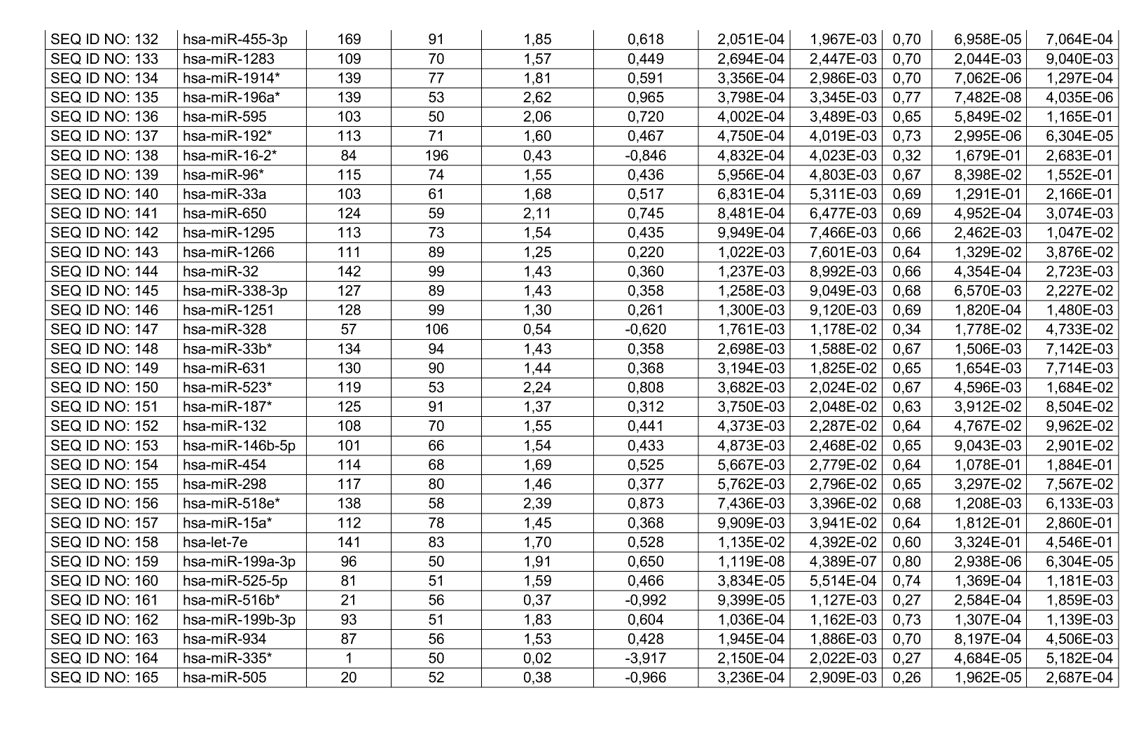

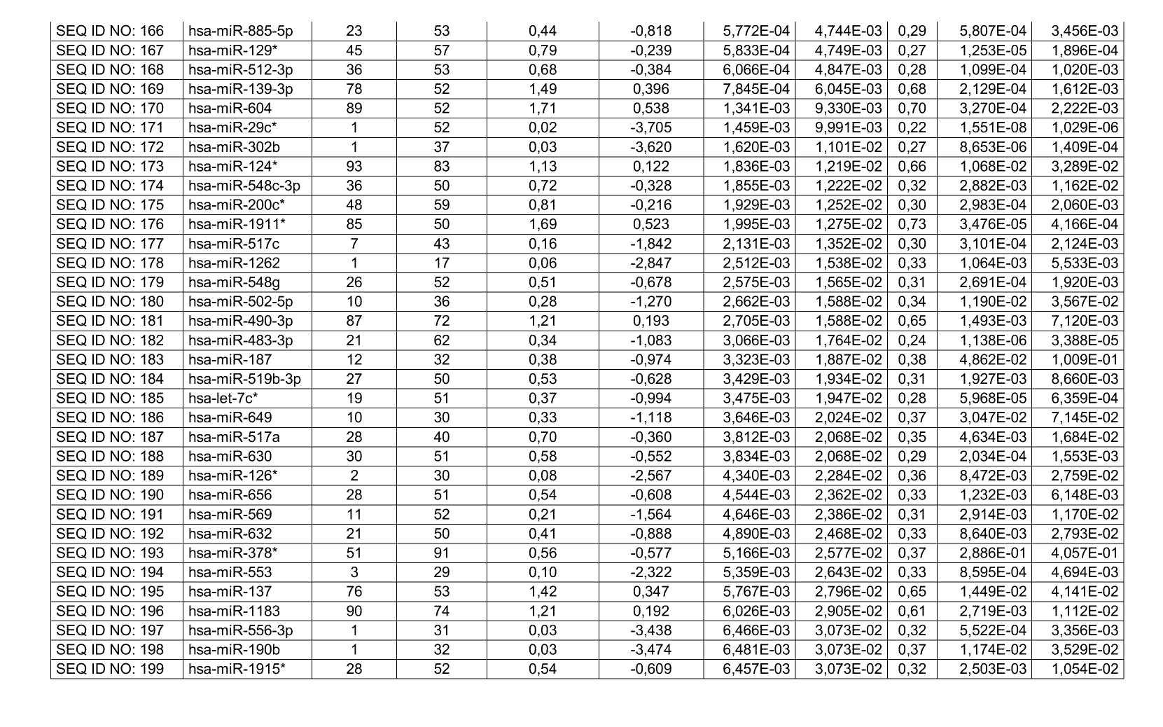

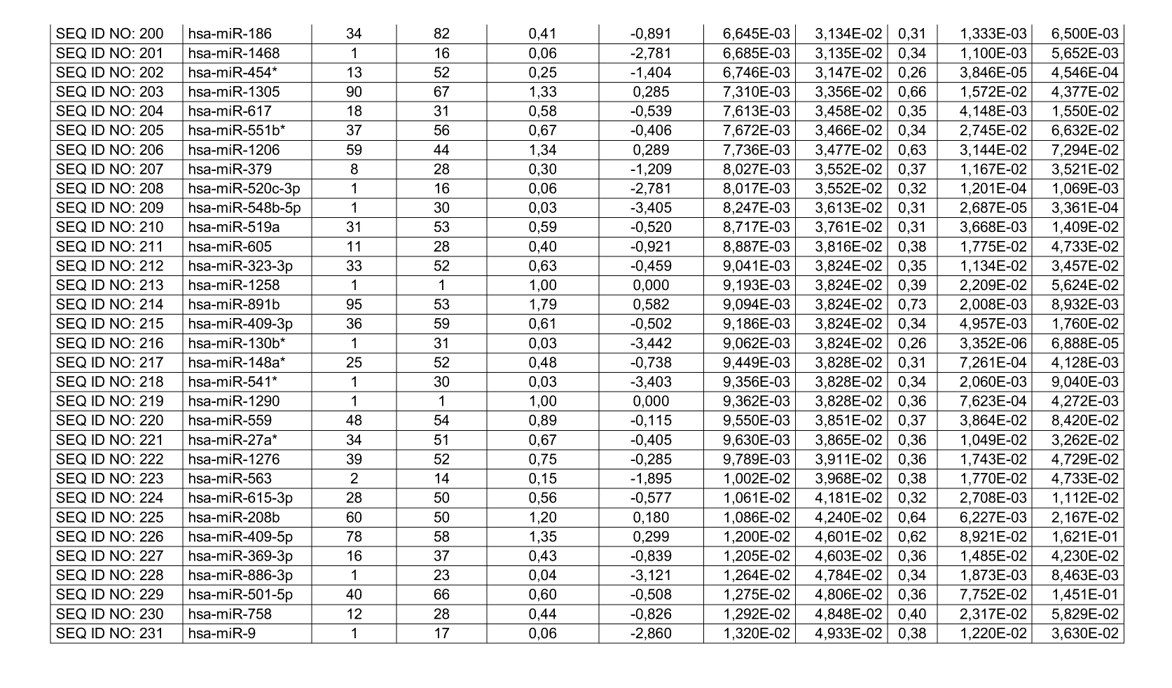

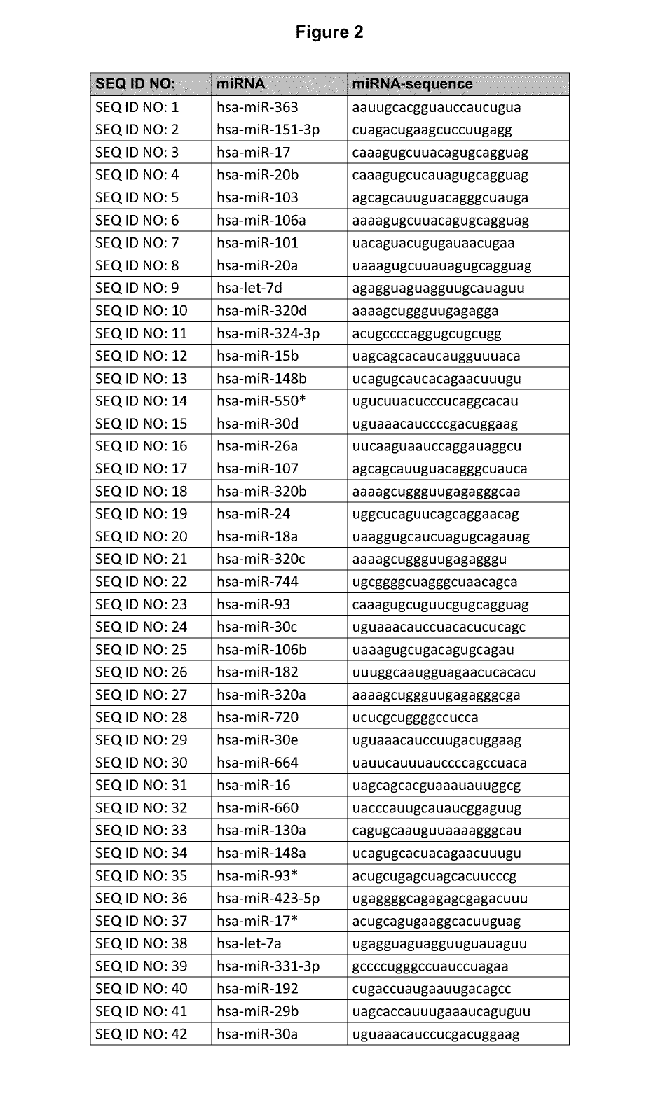

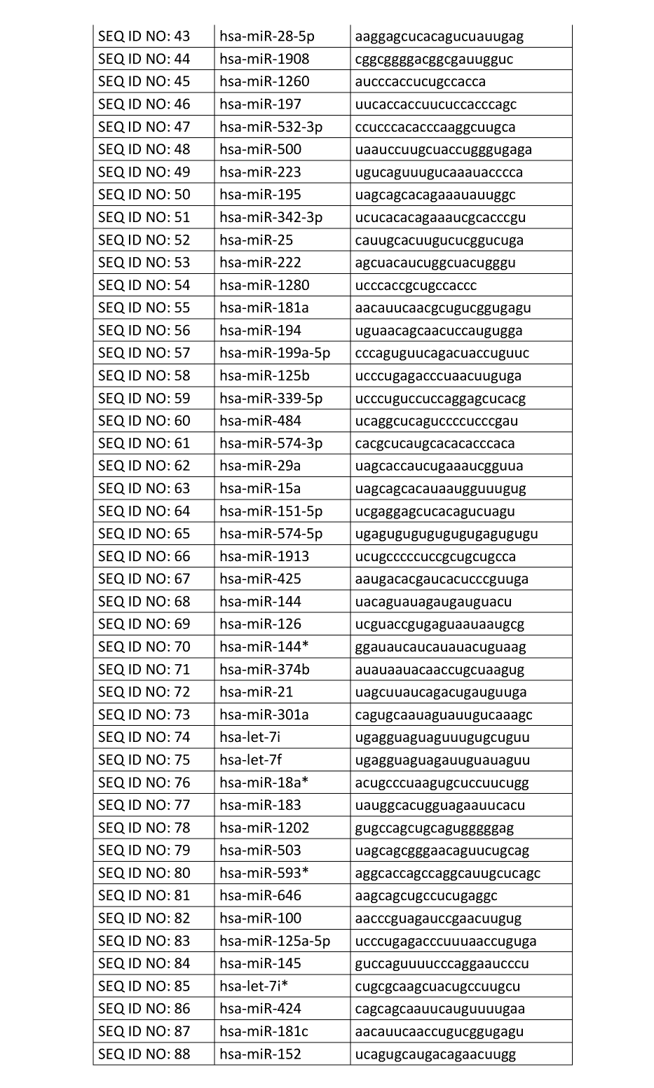

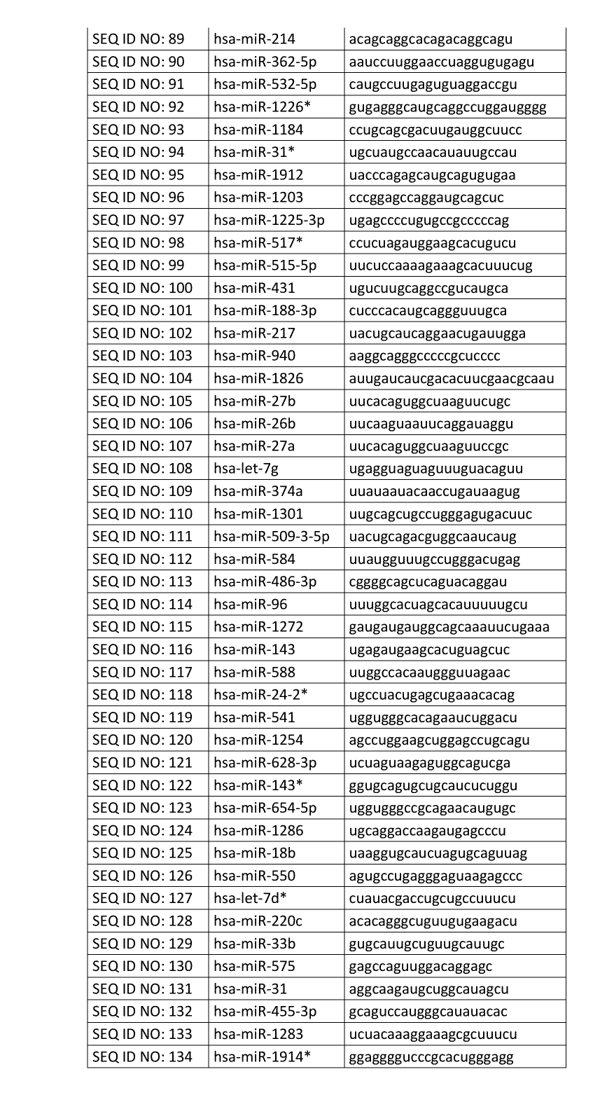

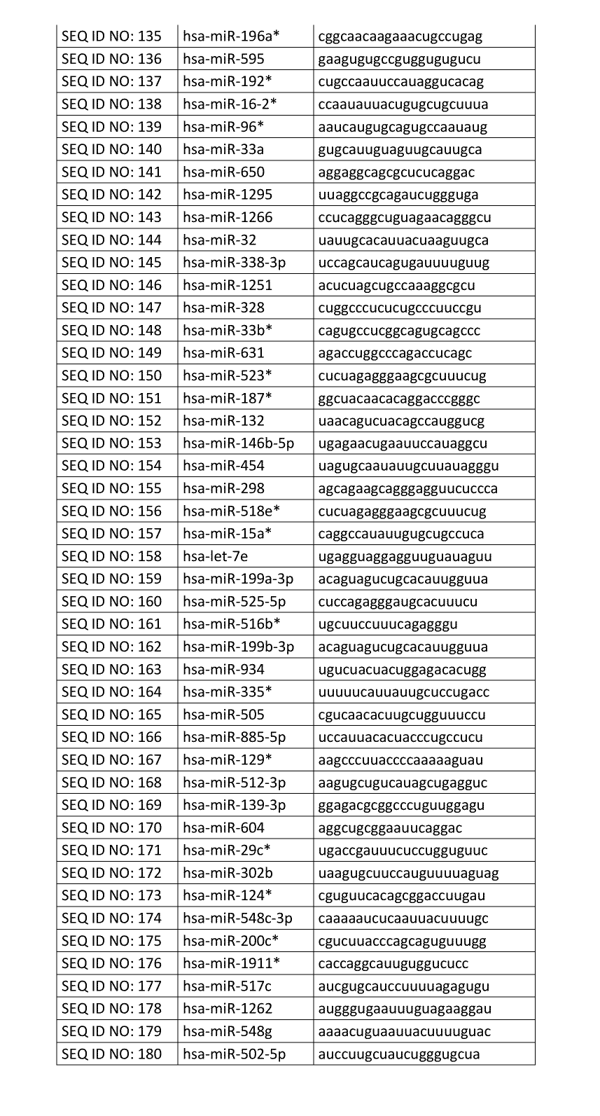

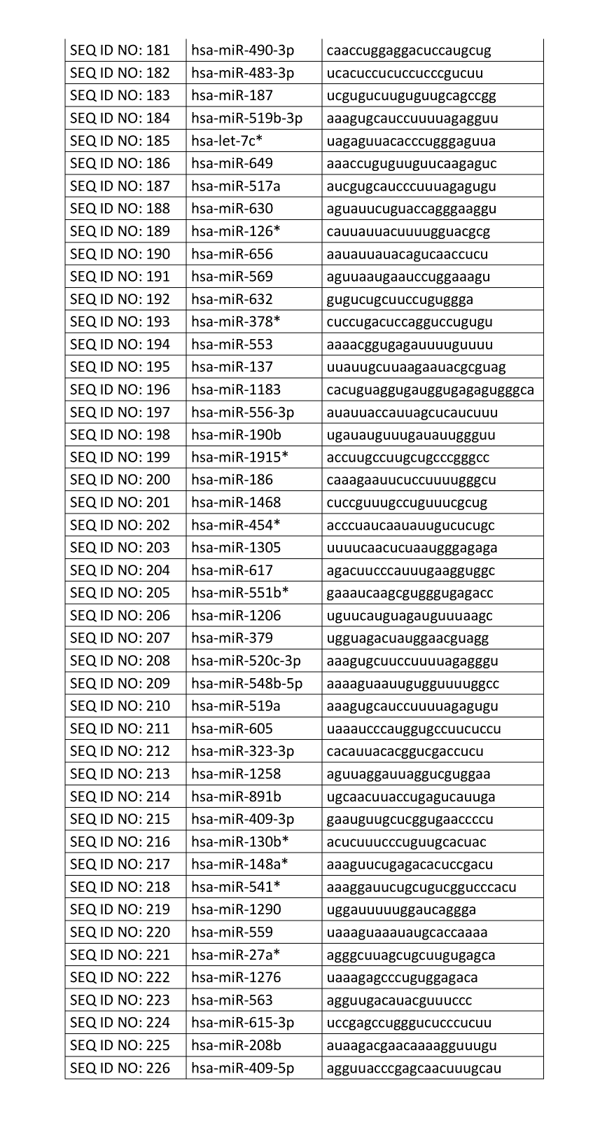

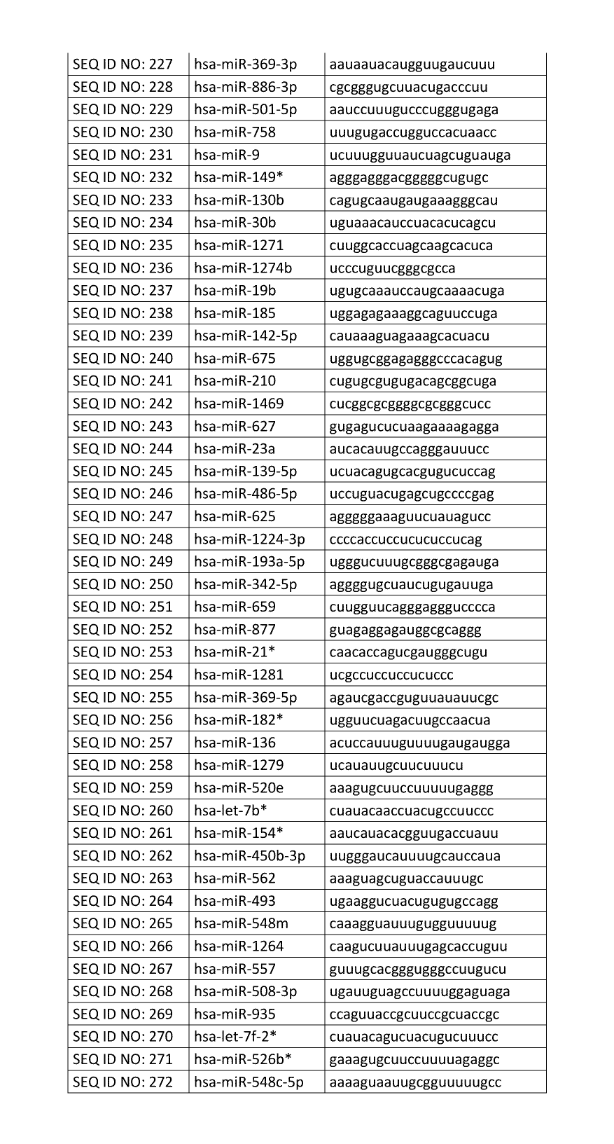

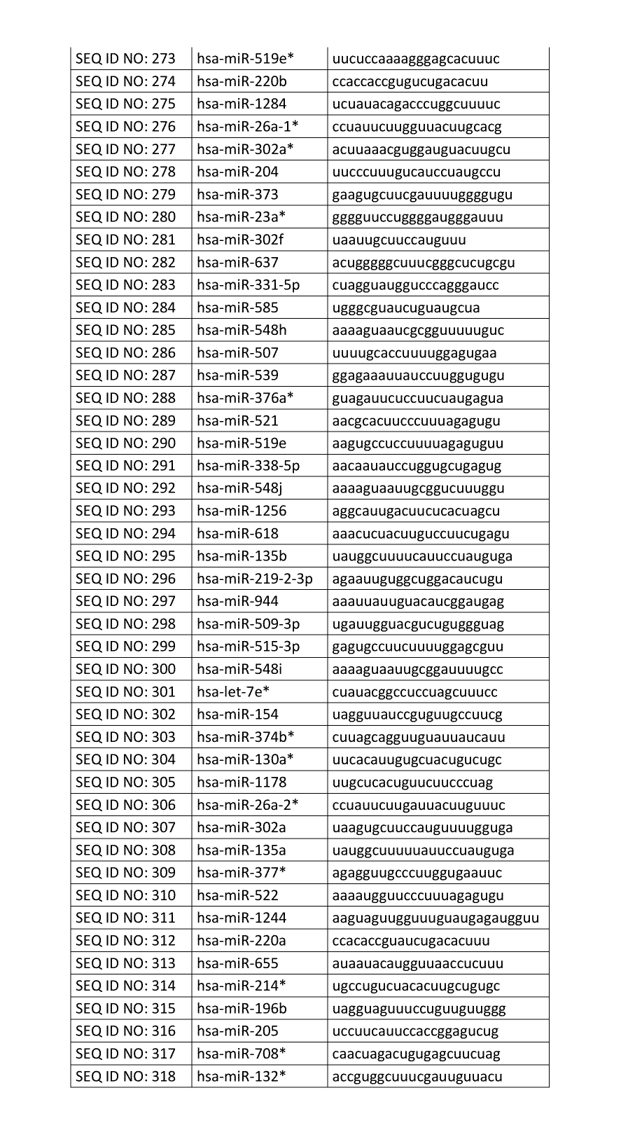

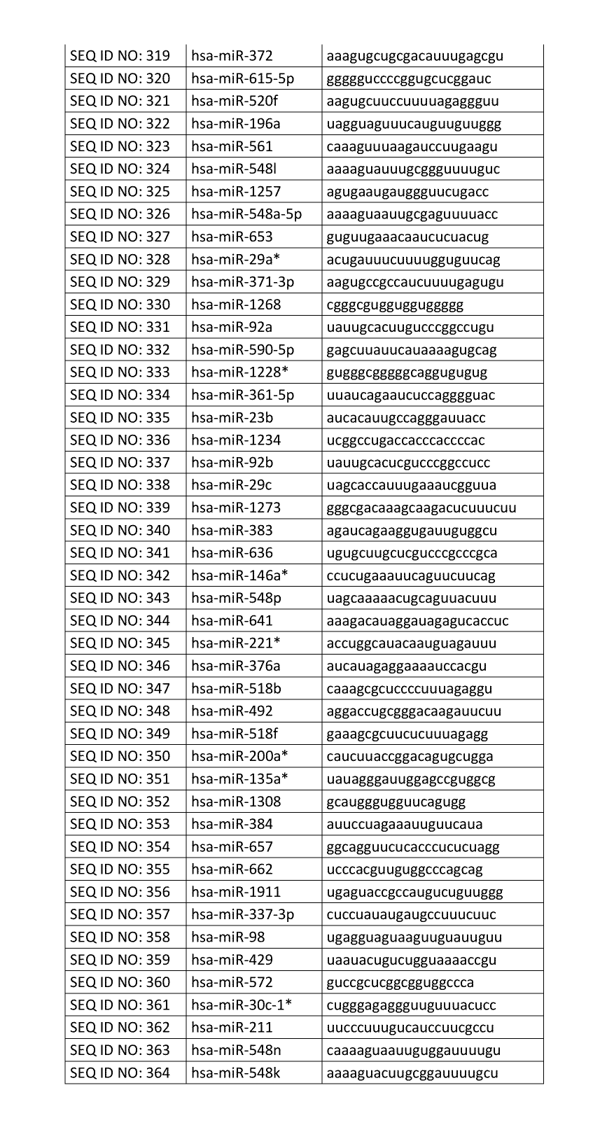

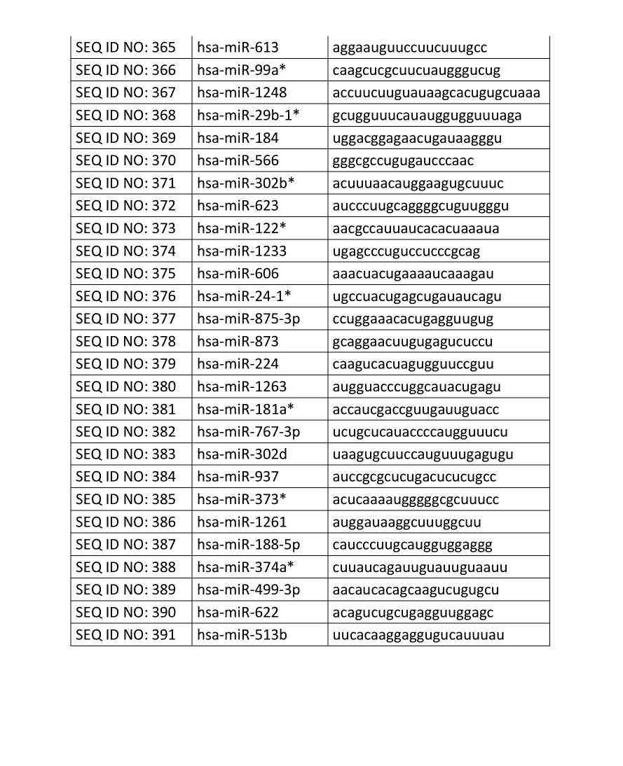

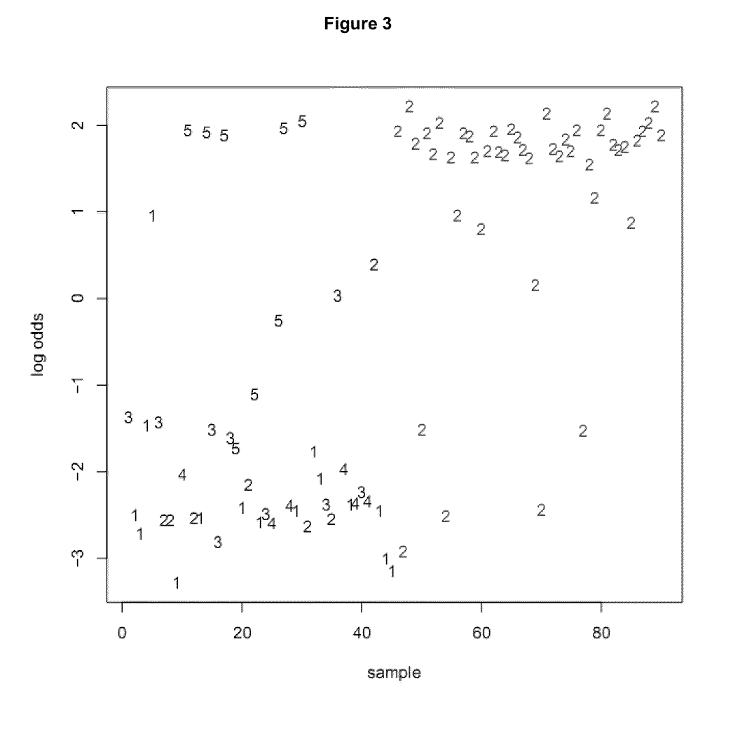

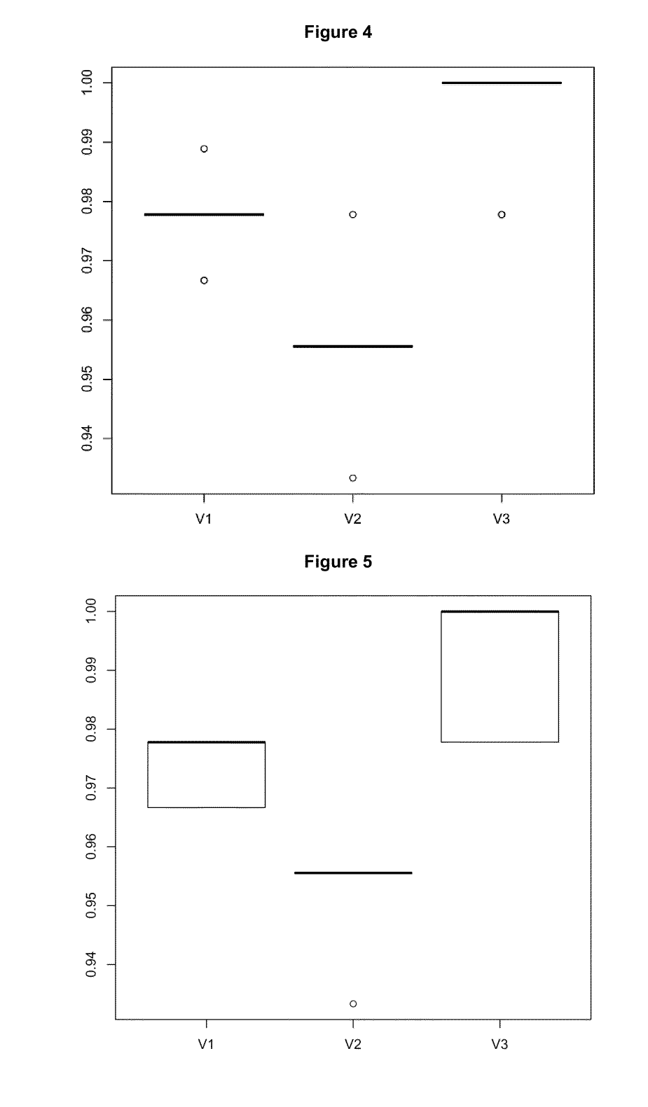

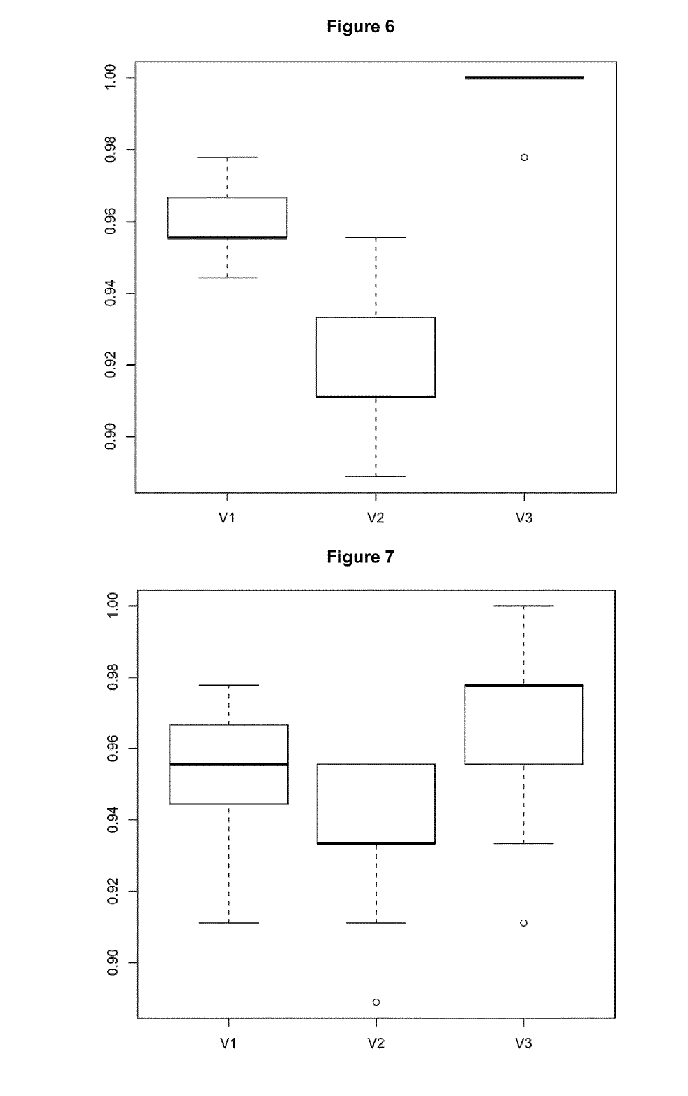

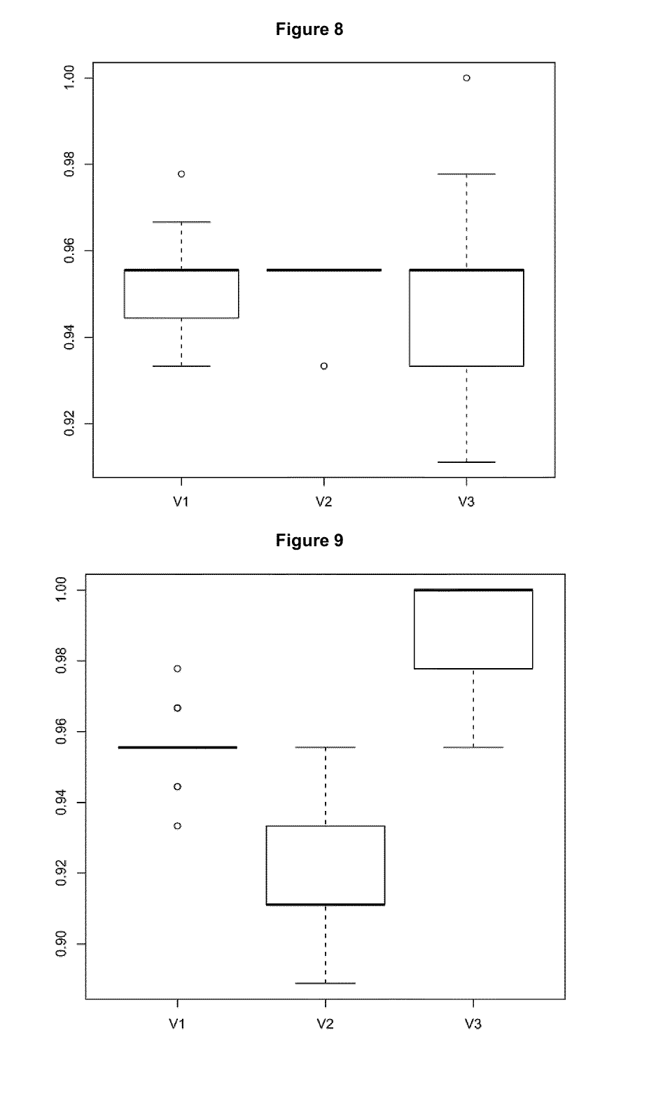

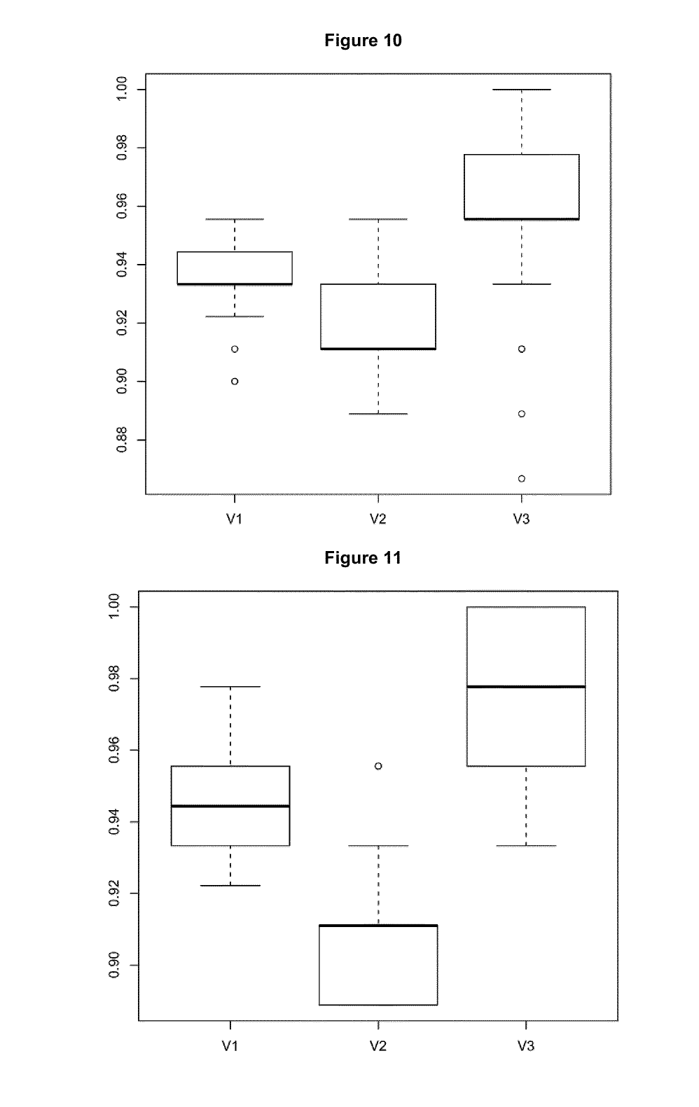

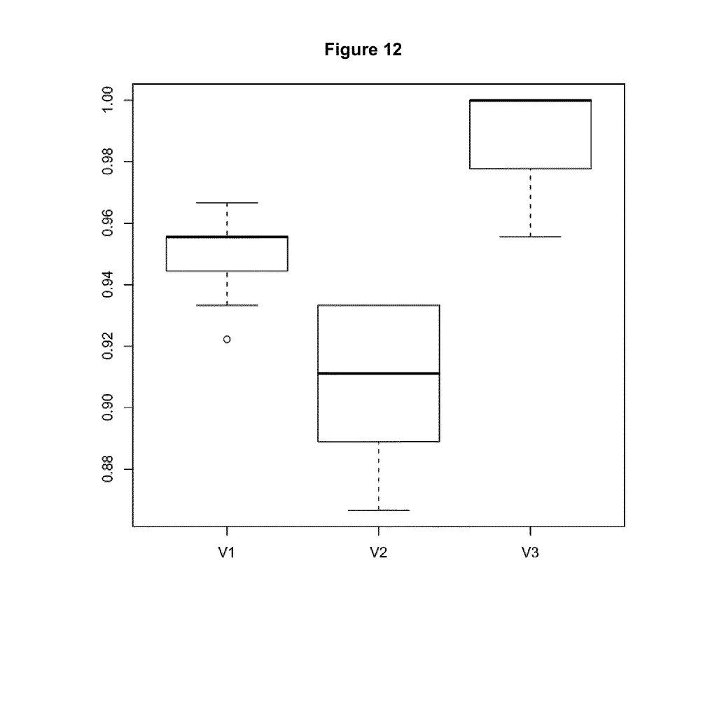

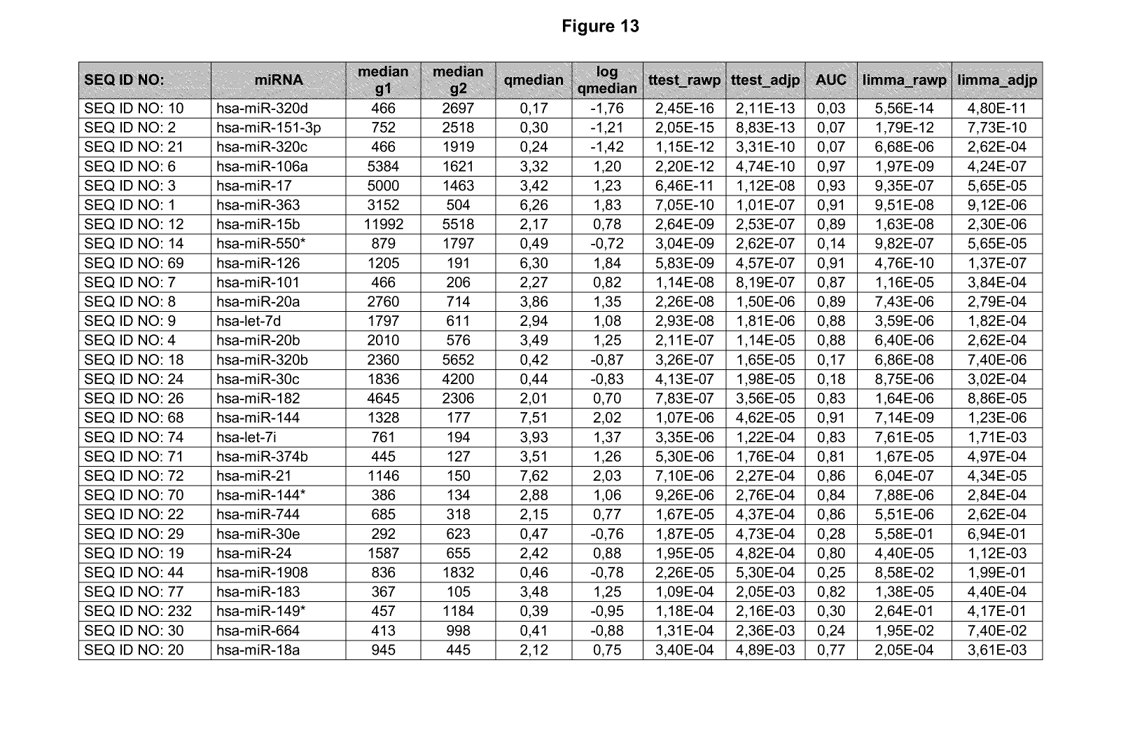

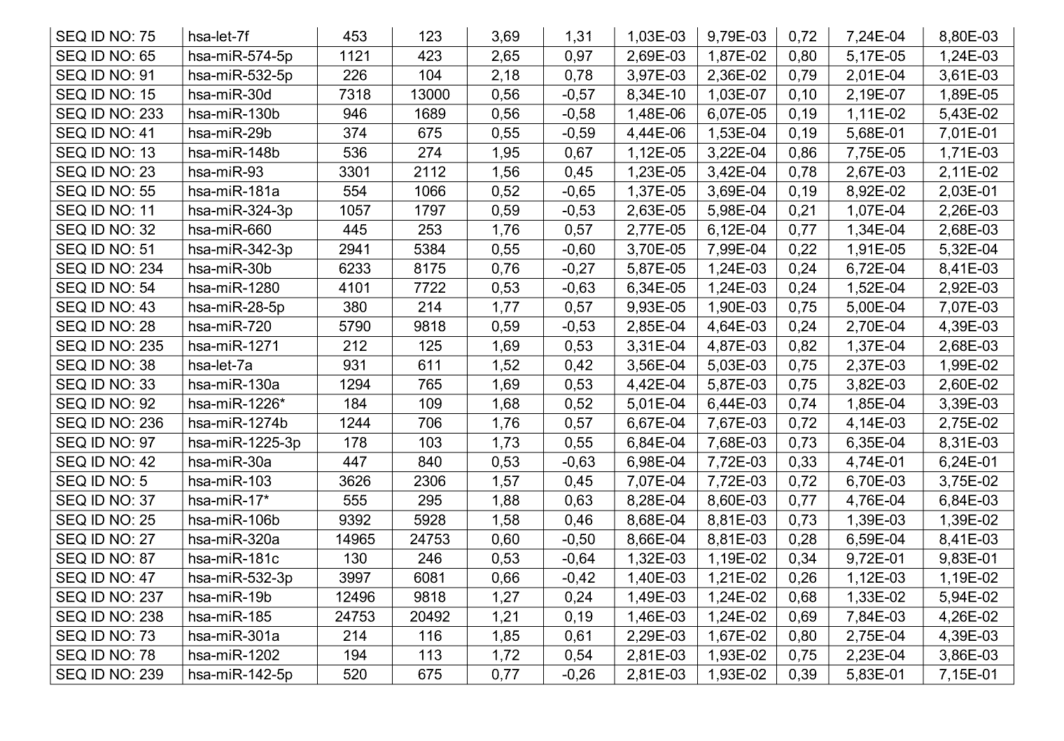

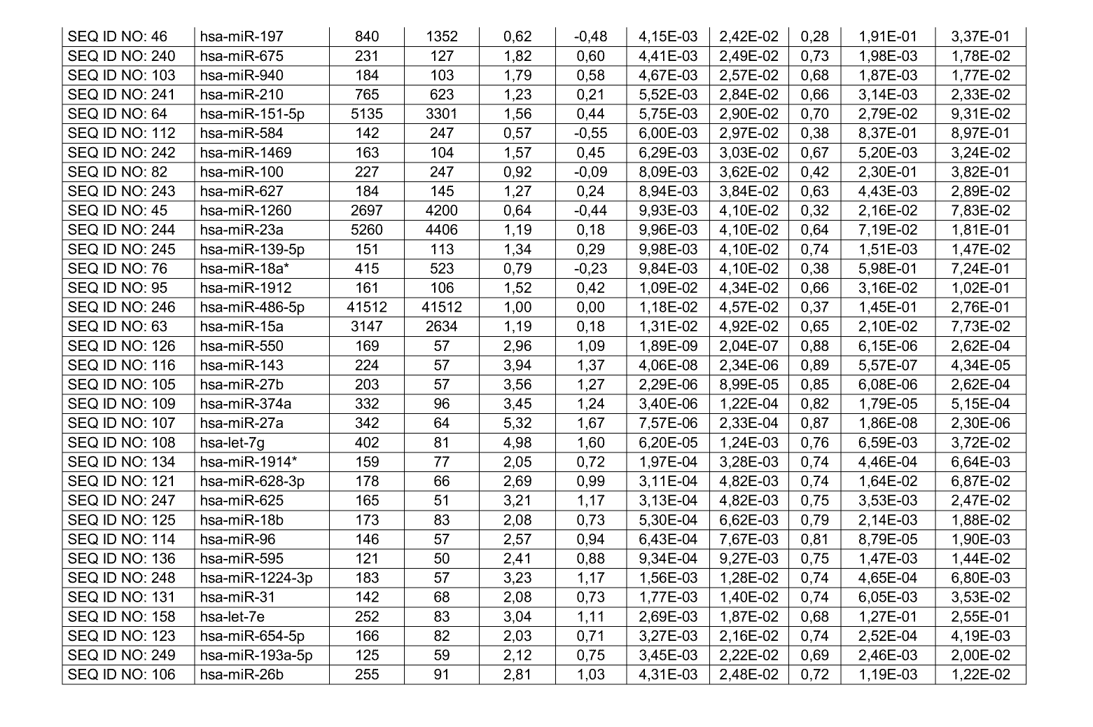

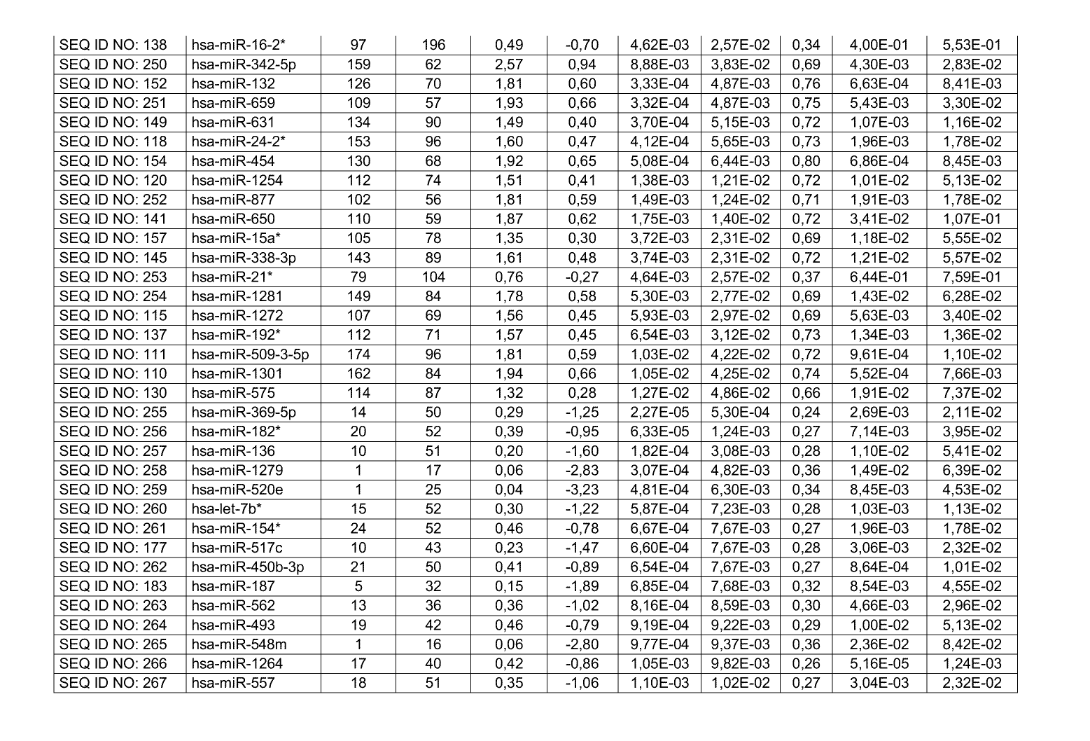

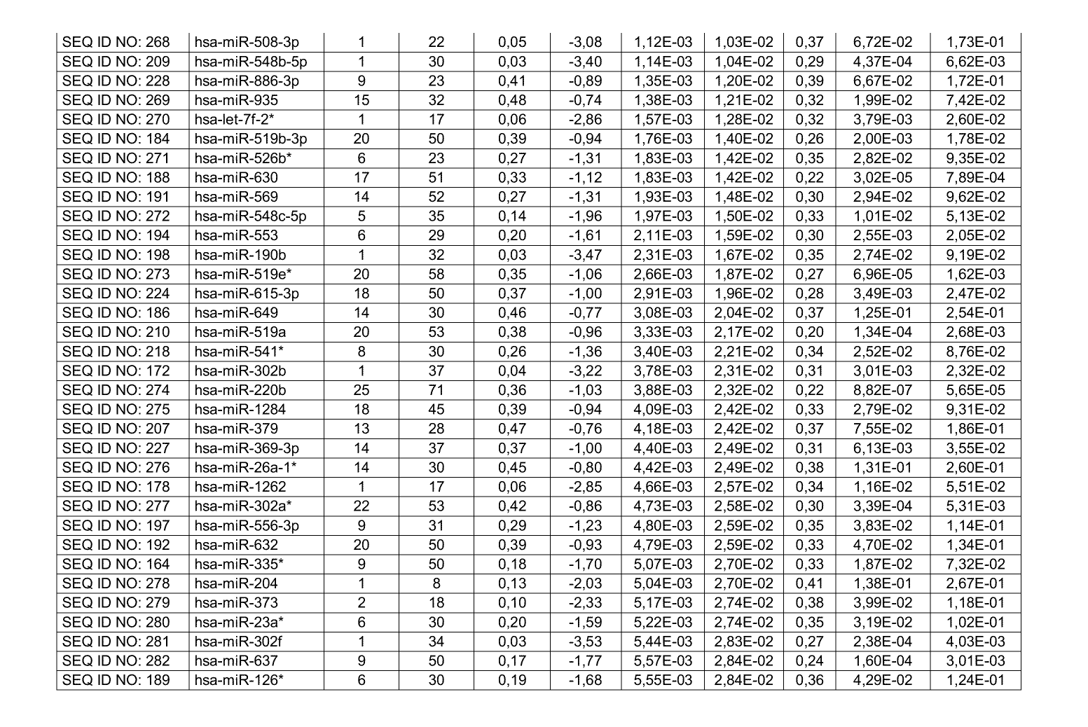

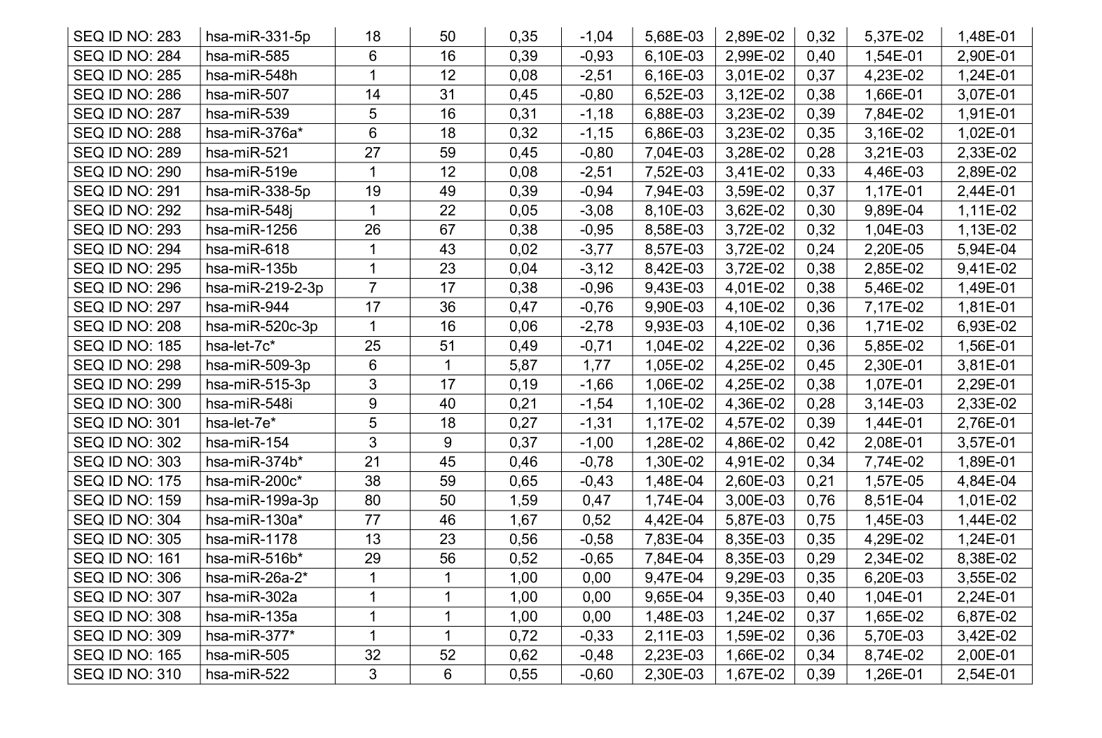

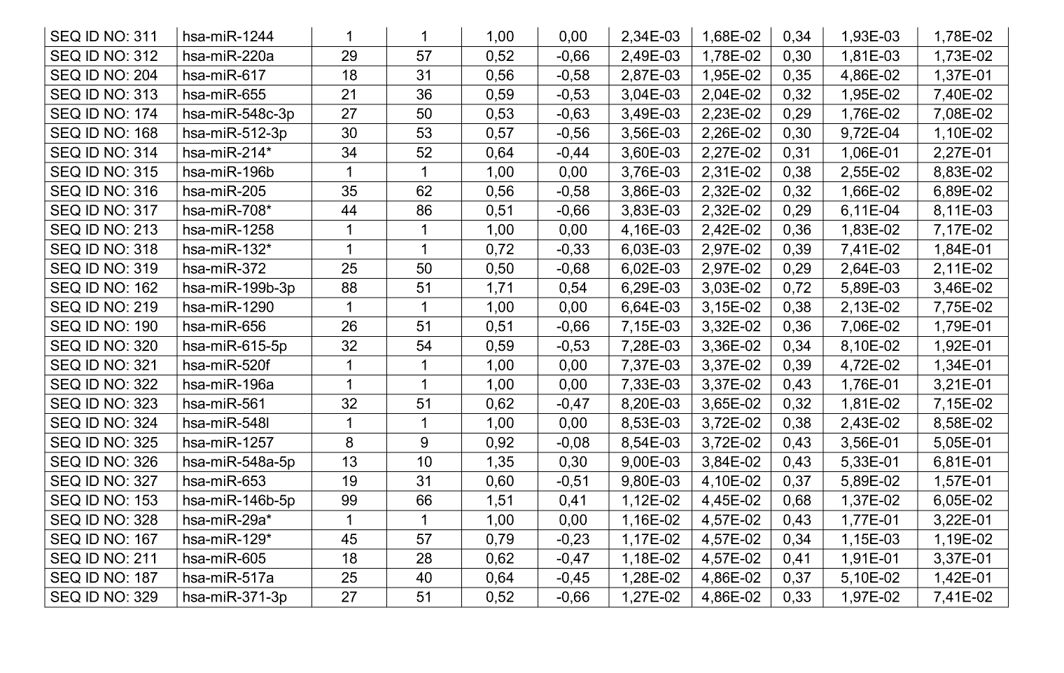

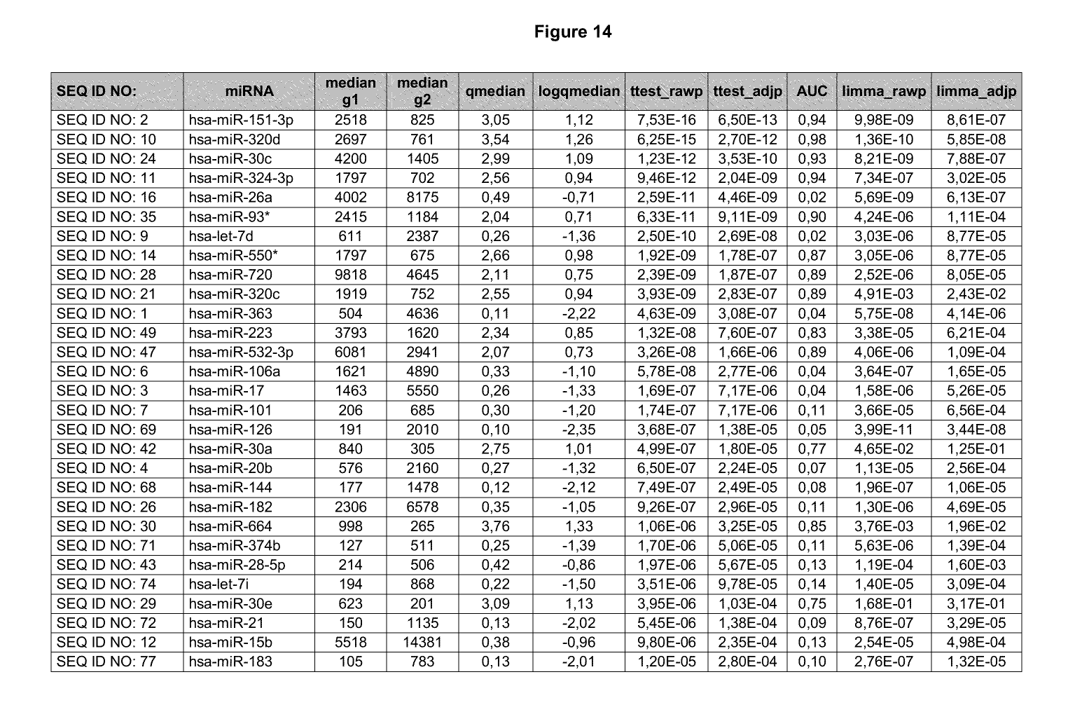

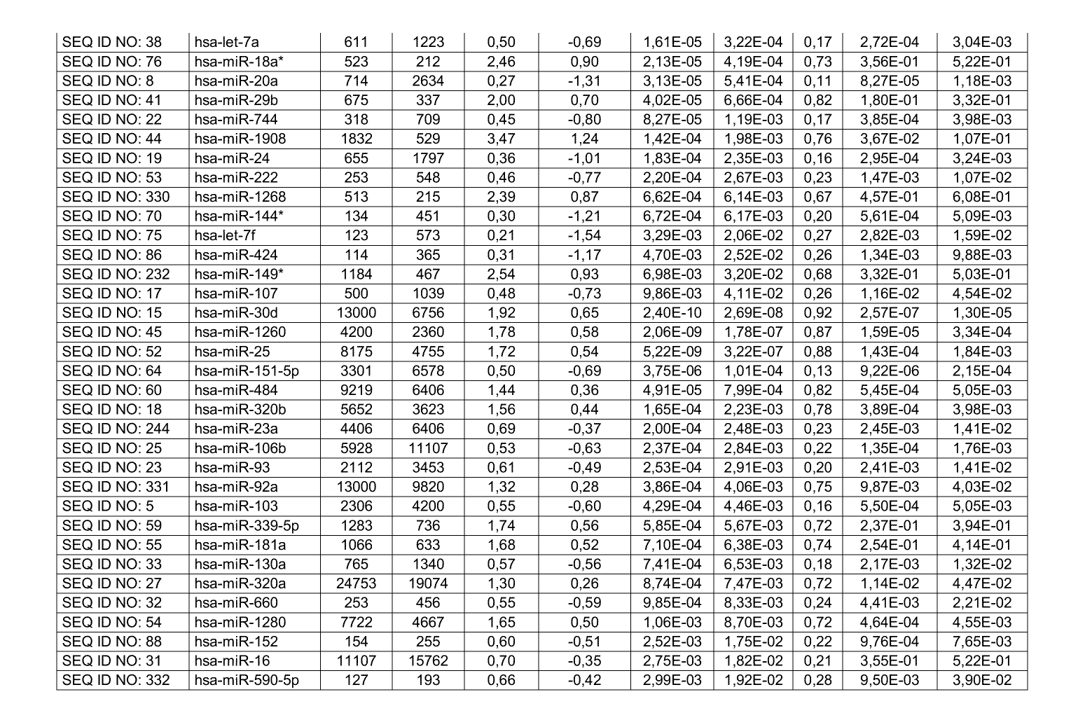

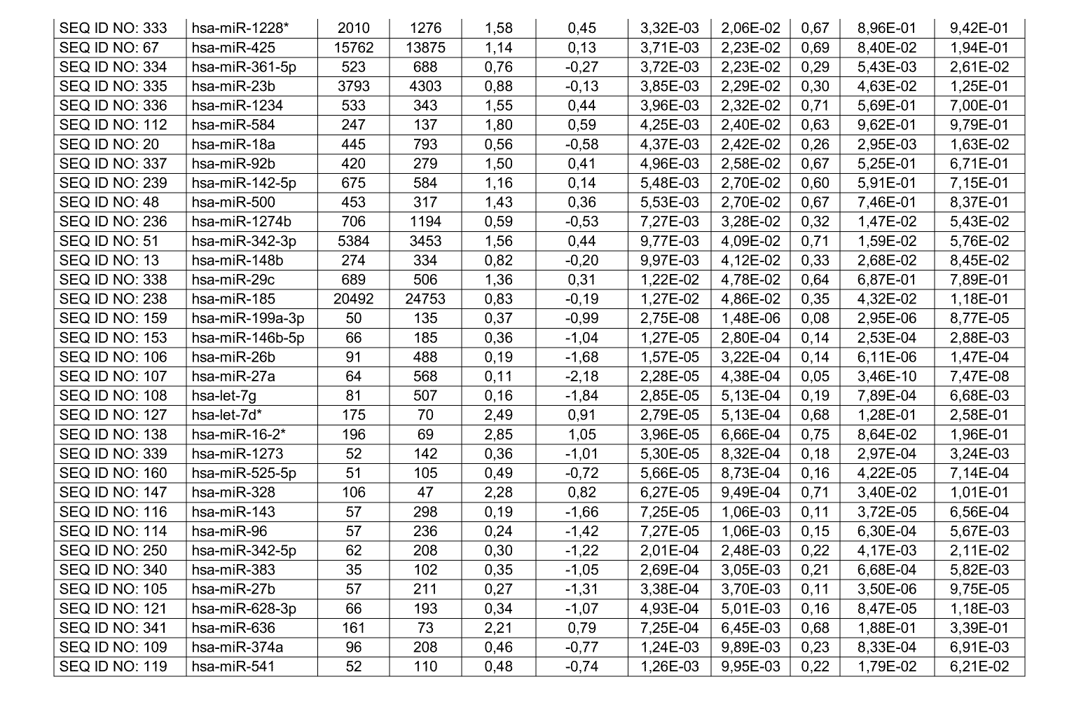

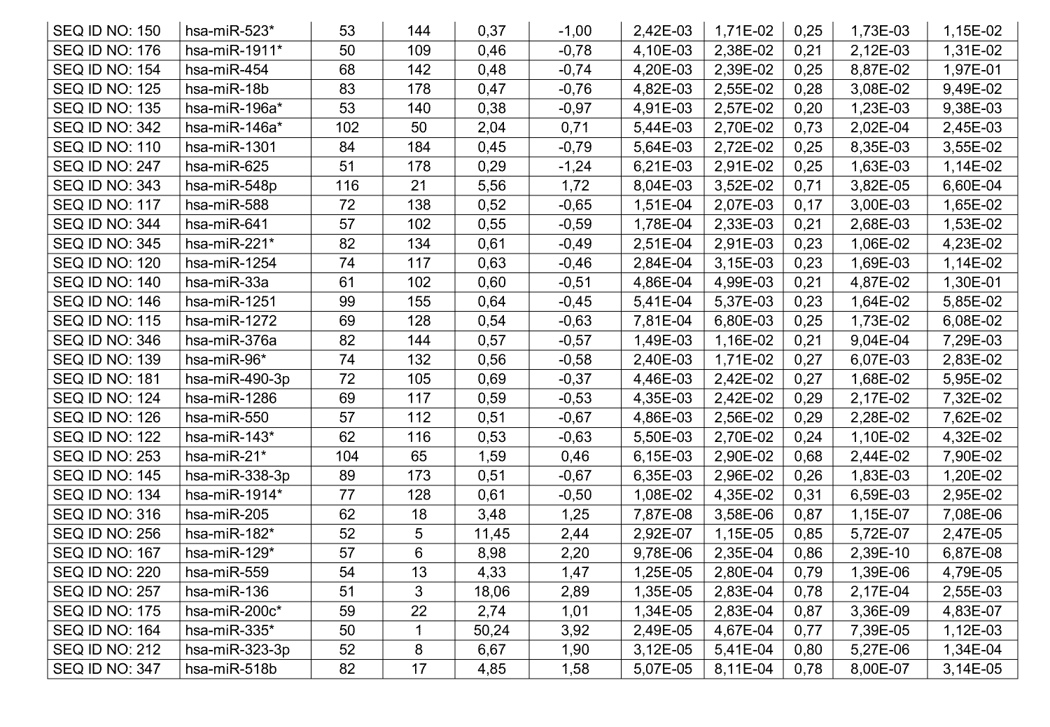

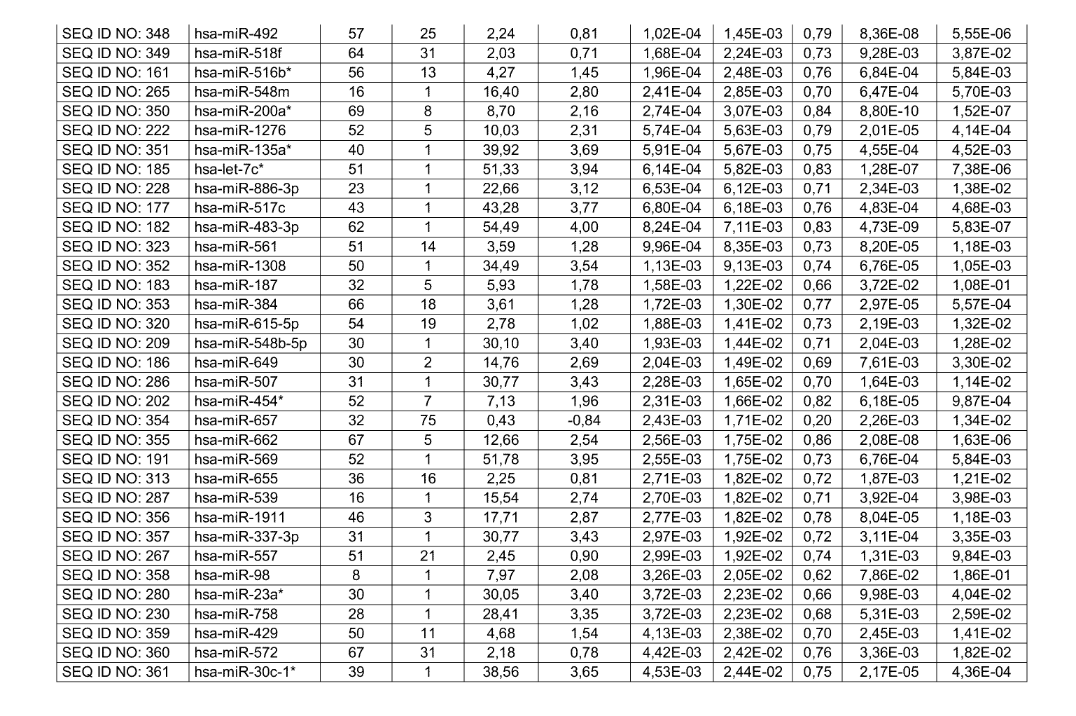

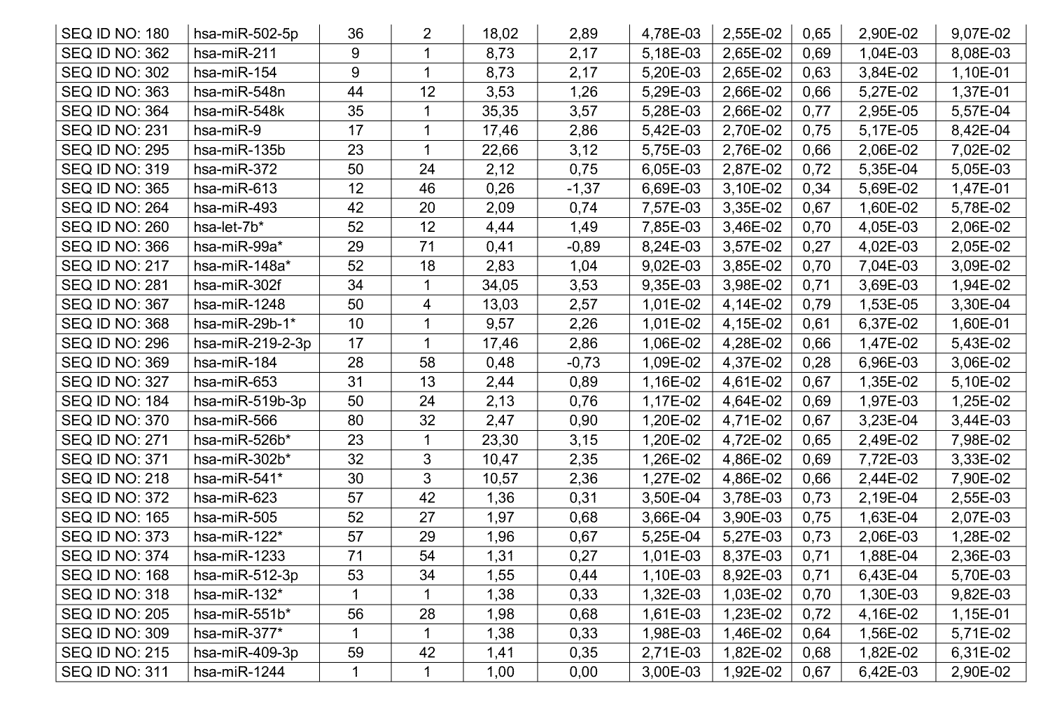

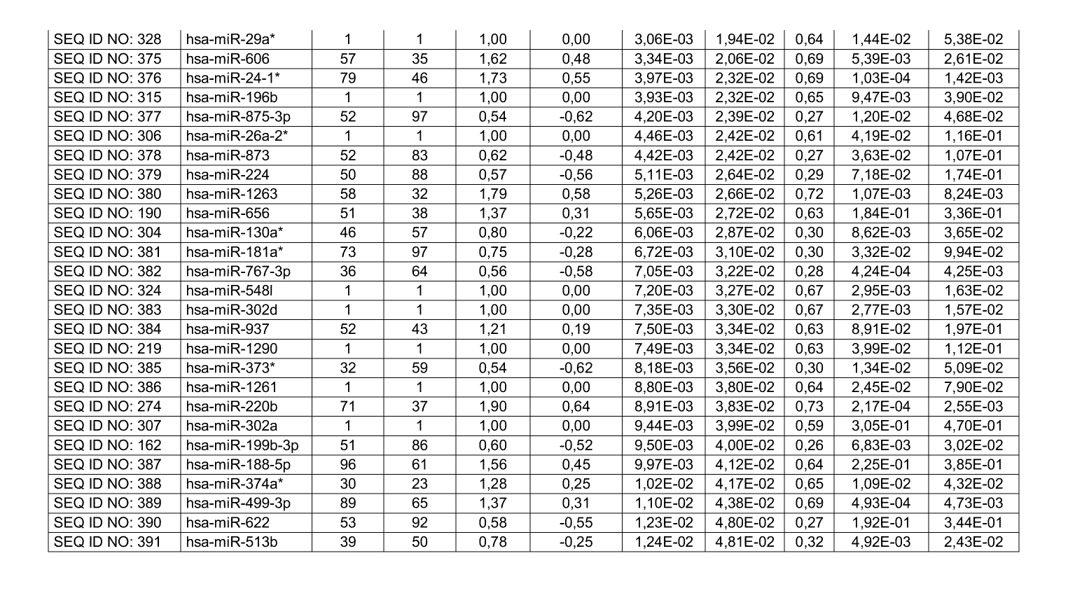

### EXAMPLES

The Examples are designed in order to further illustrate the present invention and serve a better understanding. They are not to be construed as limiting the scope of the invention in any way.

### Materials and Methods

### Samples

All blood donors participating in this study have given their written informed consent. The patient samples have been prepared at Heidelberg University. Besides the samples of diseased patients, also control samples were provided.

### MiRNA Extraction and Microarray Screening

Blood of patients has been extracted as previously described [1]. In brief, 2.5 to 5 ml blood was extracted in PAXgene Blood RNA tubes (BD, Franklin Lakes, N.J. USA) and centrifuged at 5000×g for 10 min at room temperature. The miRNeasy kit (Qiagen GmbH, Hilden) was used to isolate total RNA including miRNA from the resuspended pellet according to manufacturer's instructions. The eluted RNA was stored at −70° C.

All samples were analyzed with the Geniom RT Analyzer (febit biomed GmbH, Heidelberg, Germany) at the in-house genomic service department using the Geniom Biochip miRNA Homo sapiens. Each array contains 7 replicates of about 863 miRNAs and miRNA star sequences as annotated in the Sanger miRBase releases 12.0 to 14. On-chip sample labeling with biotin was carried out by microfluidic-based primer extension labeling of miRNAs (MPEA [2]). Following hybridization for 16 hours at 42° C., the biochip was washed and a program for signal enhancement was carried out. All steps from sample loading to miRNA detection were processed without any manual intervention and inside the machine. The detection pictures were evaluated using the Geniom Wizard Software. For each feature, the median signal intensity was calculated. Following a background correction step, the median of the 7 replicates of each miRNA was computed. To normalize the data across different arrays, quantile normalization [3] was applied and all further analyses were carried out using the normalized and background subtracted intensity values. Since the miRBase has been upgraded twice in the past year from version 12.0 to 14, we used for the final data analysis the 863 miRNAs that were consistently present in all three versions.

### Statistical Analysis

To estimate the value of single miRNAs, t-tests (unpaired, two-tailed) were carried out. The resulting p-values have been adjusted for multiple testing by Benjamini-Hochberg adjustment [4, 5].

In addition to this single biomarker analysis, we performed supervised classification of samples by using Support Vector Machines (SVM [6]) as implemented in the Re1071 package [7]. As parameters, we evaluated different kernel methods including linear, polynomial (degree 2 to 5), sigmoid and radial basis function kernels. The cost parameter was sampled from 0.01 to 10 in decimal powers. As subset selection technique, a filter approach based on t-test was carried out. In each iteration, the s miRNAs with lowest p-values were computed on the training set in each fold of a standard 10-fold cross validation, where s was sampled in regular intervals between 2 and 300. The respective subset was used to train the SVM and to carry out the prediction of the test samples in the cross validation. To compute probabilities for classes instead of class labels, a regression approach based on the output of the support vectors has been applied. To test for overtraining, non-parametric permutation tests have been applied. All computations were carried out using R [7], a freely available language for statistical tasks.

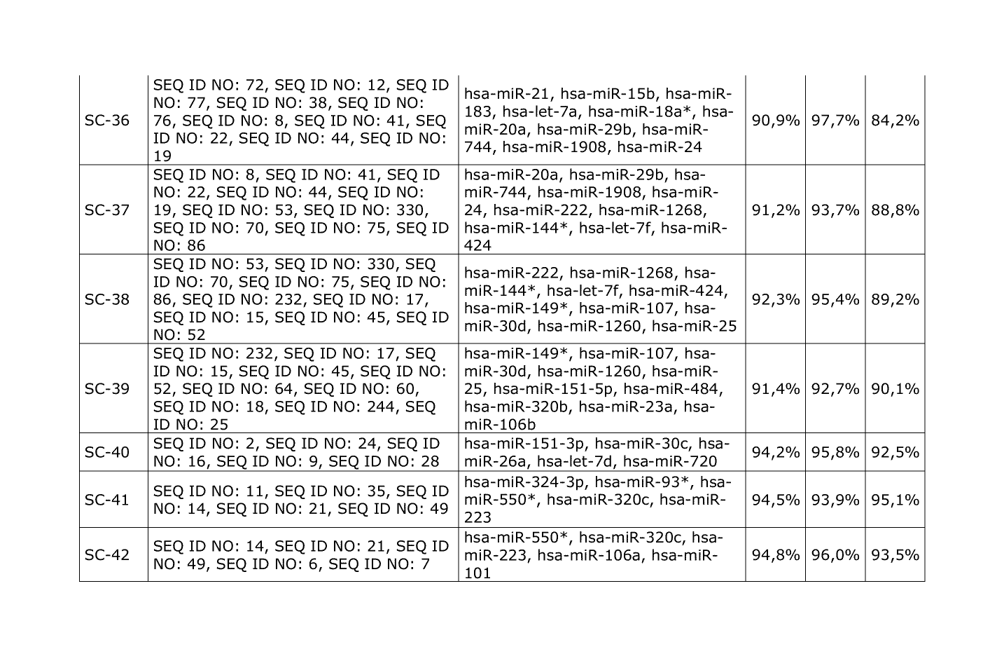

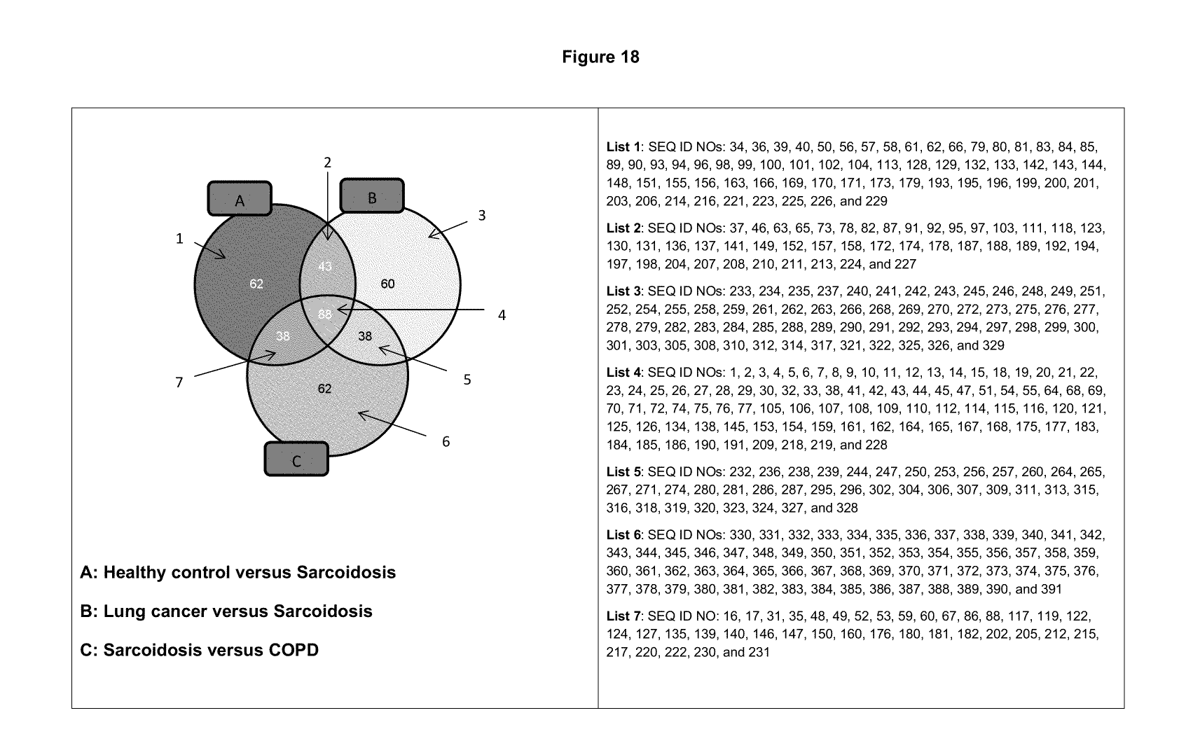

For all analysis, i.e. healthy versus sarcoidosis, lung cancer versus sarcoidosis, and sarcoidosis versus COPD, the same protocol (as described above) has been used (see also FIG. 18 and legend to FIG. 18).

The results are shown in the Figures.

### REFERENCES

1. Keller A, Leidinger P, Borries A, Wendschlag A, Wucherpfennig F, Scheffler M, Huwer H, Lenhof H P, Meese E: miRNAs in lung cancer—studying complex fingerprints in patient's blood cells by microarray experiments. BMC Cancer 2009, 9:353.
2. Vorwerk S, Ganter K, Cheng Y, Hoheisel J, Stahler P F, Beier M: Microfluidic-based enzymatic on-chip labeling of miRNAs. N Biotechnol 2008, 25(2-3):142-149.
3. Bolstad B M, Irizarry R A, Astrand M, Speed T P: A comparison of normalization methods for high density oligonucleotide array data based on variance and bias. Bioinformatics 2003, 19(2):185-193.
4. Benjamini Y, Drai D, Elmer G, Kafkafi N, Golani I: Controlling the false discovery rate in behavior genetics research. Behav Brain Res 2001, 125(1-2):279-284.
5. Hochberg Y: A sharper bonferroni procedure for multiple tests of significance. Biometrica 1988, 75:185-193.
6. Vapnik V: The nature of statistical learning theory, 2nd edition edn. New York: Spinger; 2000.
7. Team R: R: A Language and Environment for Statistical Computing. In. Vienna: R Foundation for Statistical Computing; 2008.

1. One or more polynucleotide(s) for detecting one or more miRNA(s) for diagnosing and/or prognosing of sarcoidosis in a blood sample from a patient, wherein the nucleotide sequence of said miRNA or the nucleotide sequences of said miRNAs is (are) selected from the group consisting of SEQ ID NOs: 1 to 231 (lists 1, 2, 4 and 7 of FIG. 18).

2. (canceled)

3. The polynucleotide(s) of claim 1, wherein the nucleotide sequence of said miRNA or the nucleotide sequences of said miRNAs is (are) selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (list 1 of FIG. 18).

4. The polynucleotide(s) of claim 1, wherein said polynucleotide(s) is (are) for detecting one or more miRNA(s) for differential diagnosis with respect to lung cancer and/or COPD, and wherein the nucleotide sequence of said miRNA or the nucleotide sequences of said miRNAs is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (list 4 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (list 7 of FIG. 18).

5. (canceled)

6. The polynucleotide(s) of claim 4, wherein said polynucleotide(s) is (are) for detecting one or more miRNA(s) for differential diagnosis with respect to lung cancer and COPD, and wherein the nucleotide sequence of said miRNA or the nucleotide sequences of said miRNAs is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (list 4 of FIG. 18).

7. (canceled)

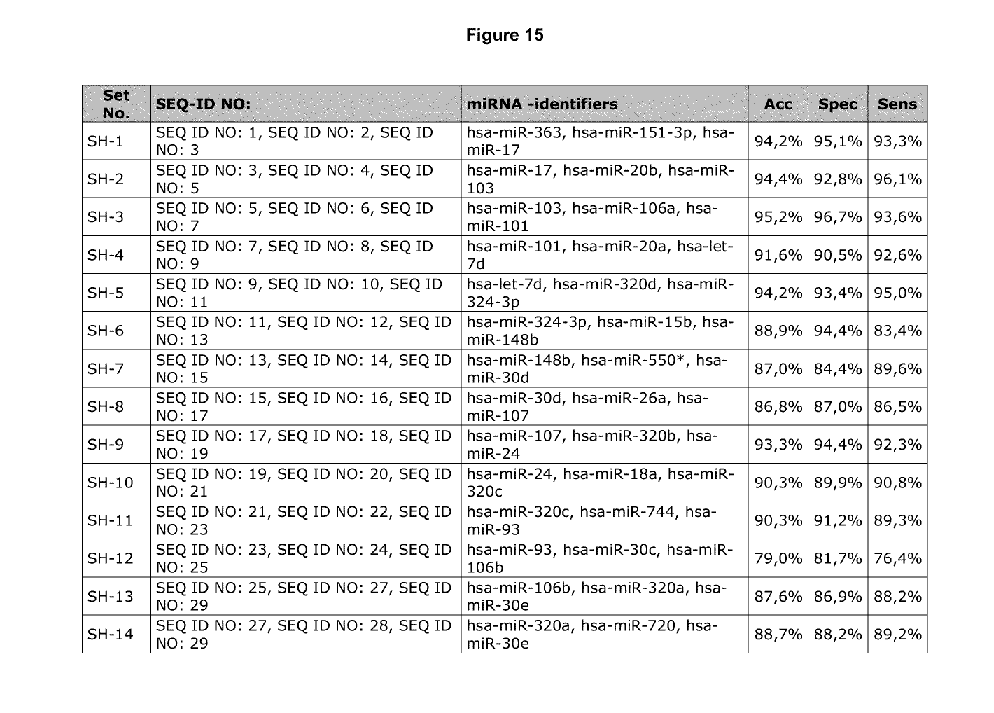

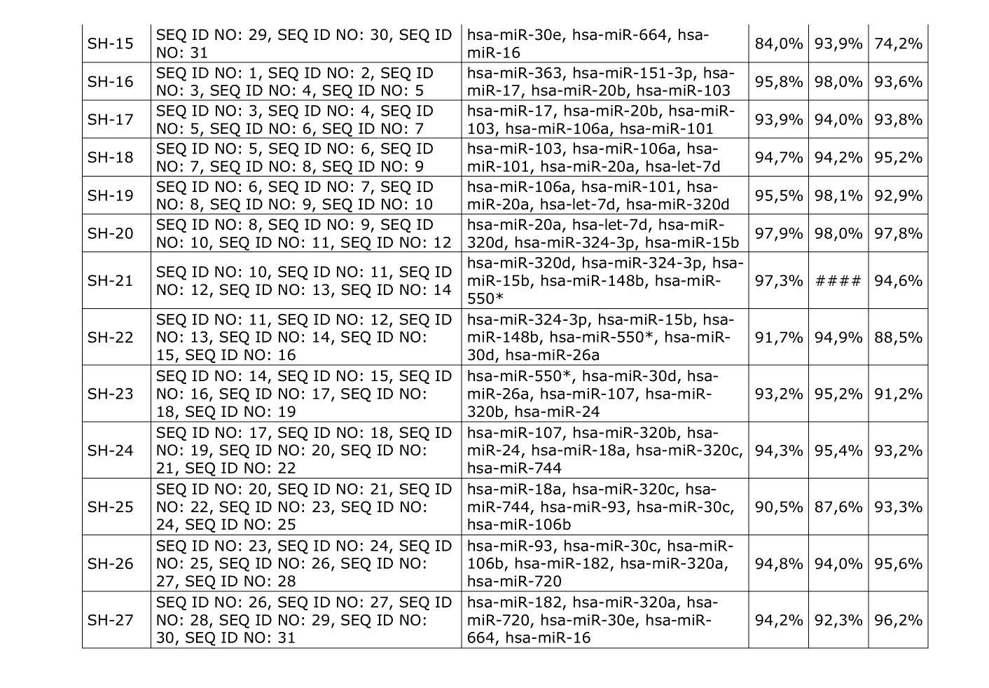

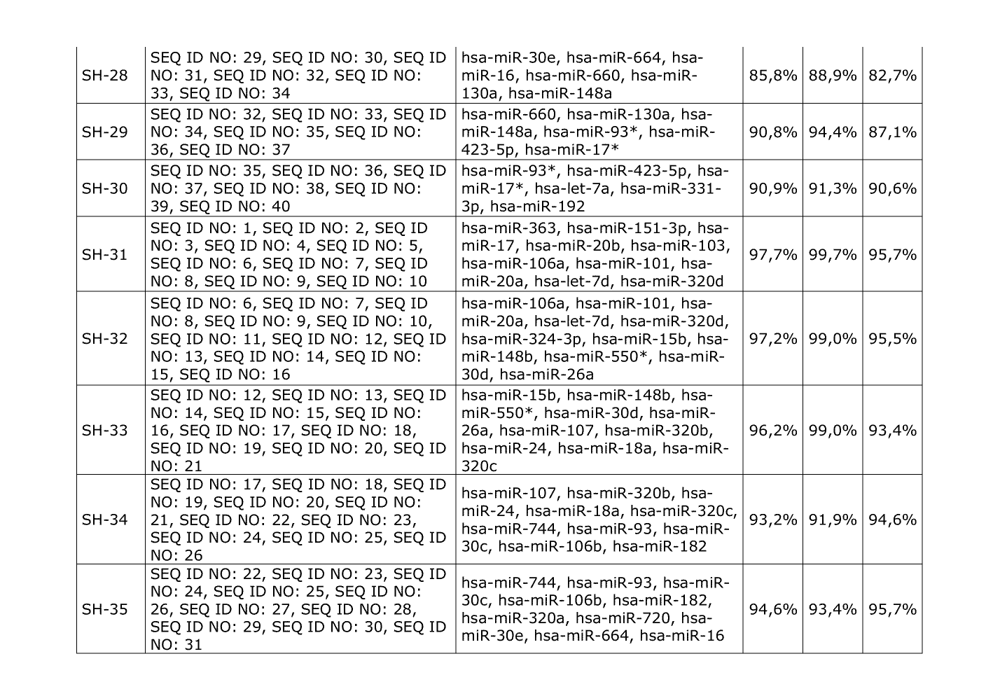

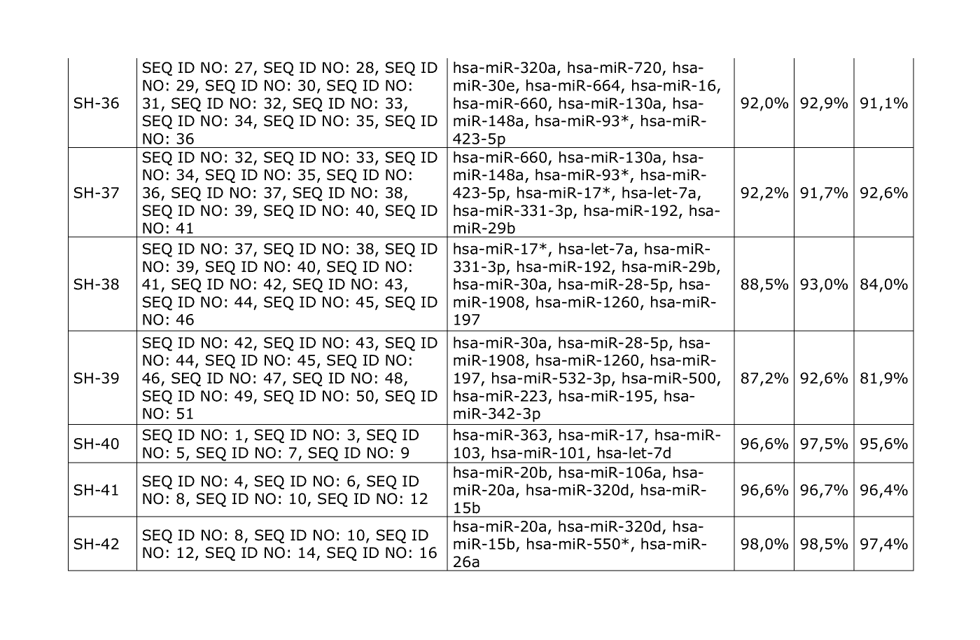

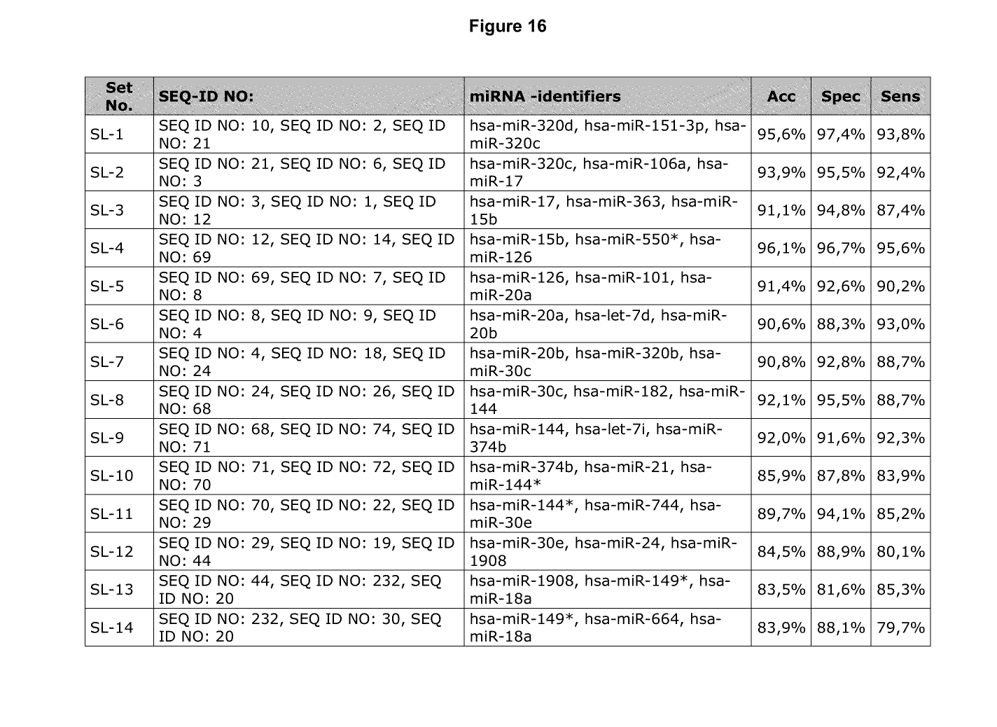

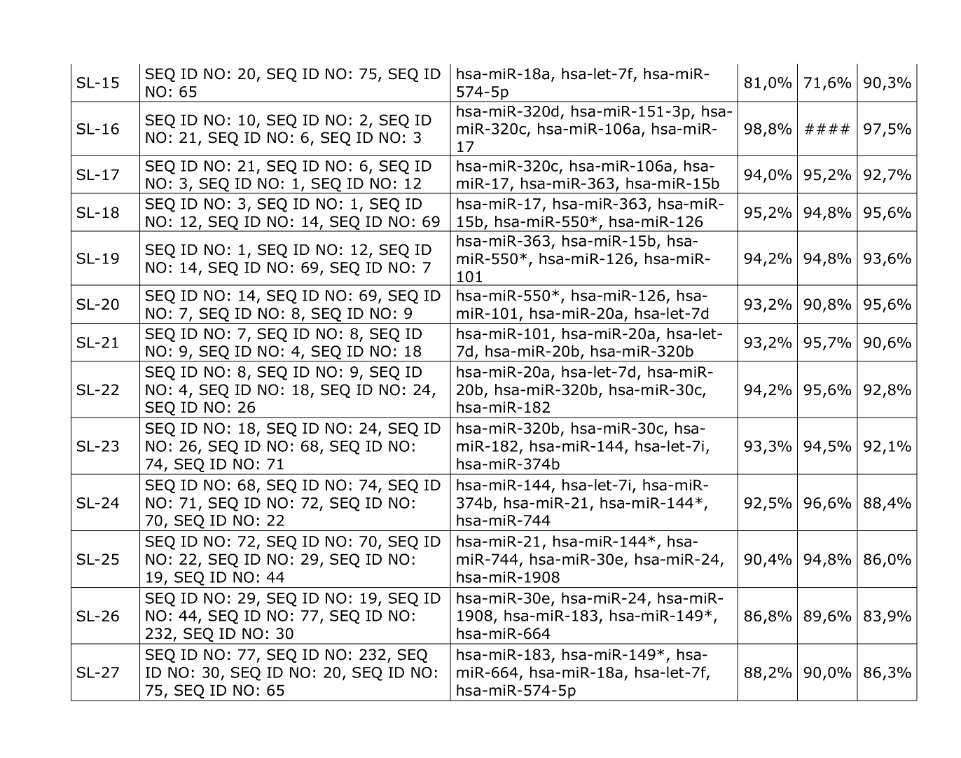

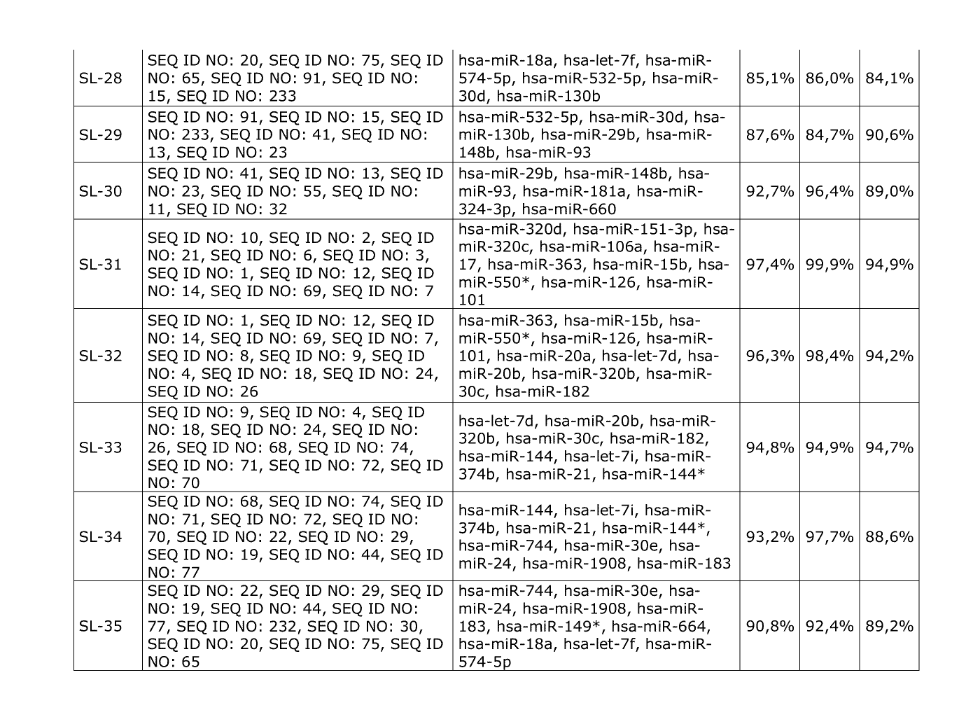

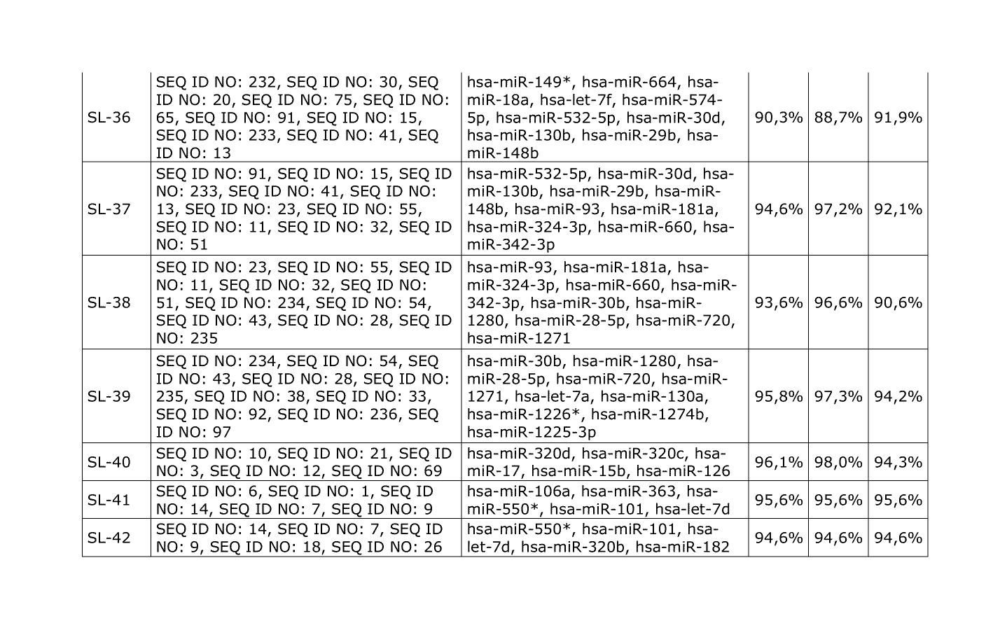

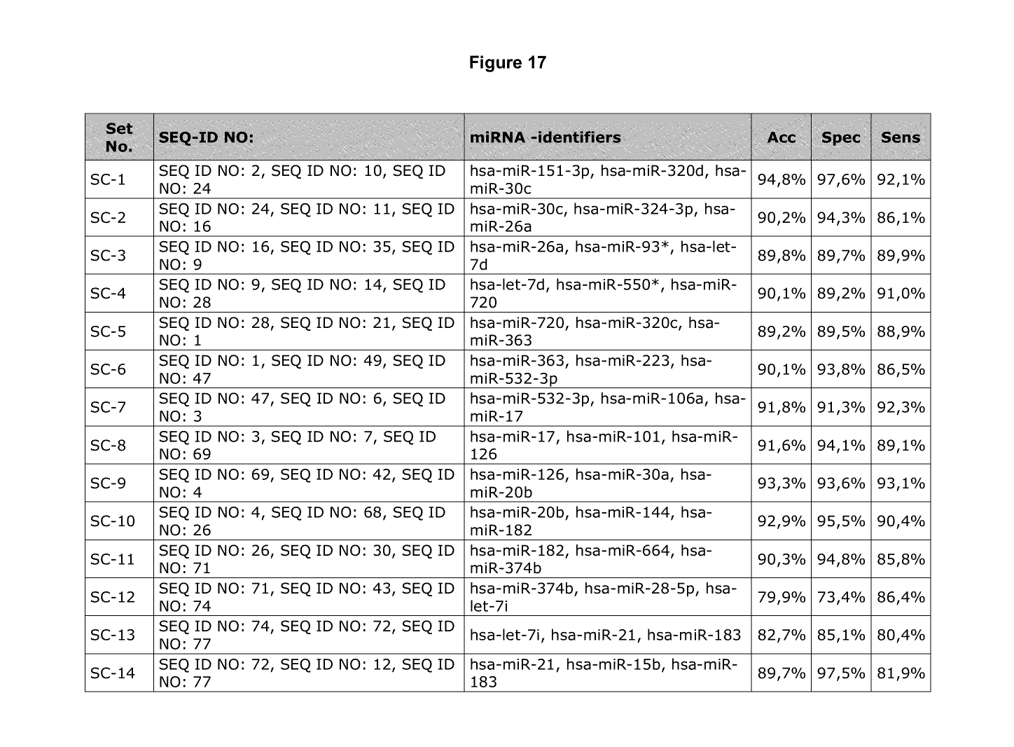

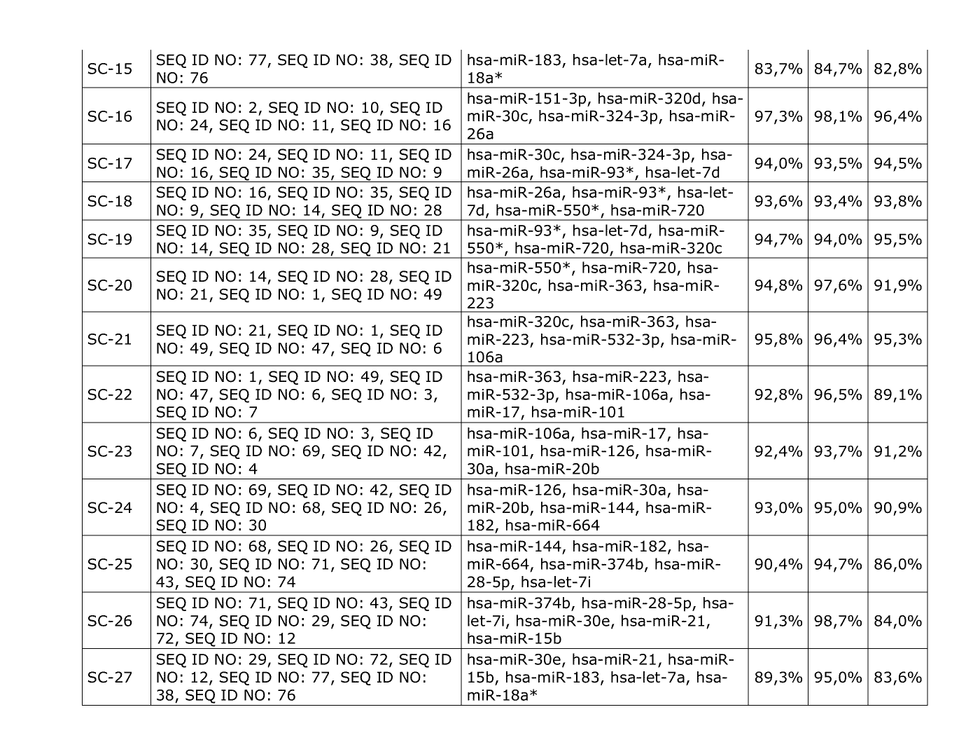

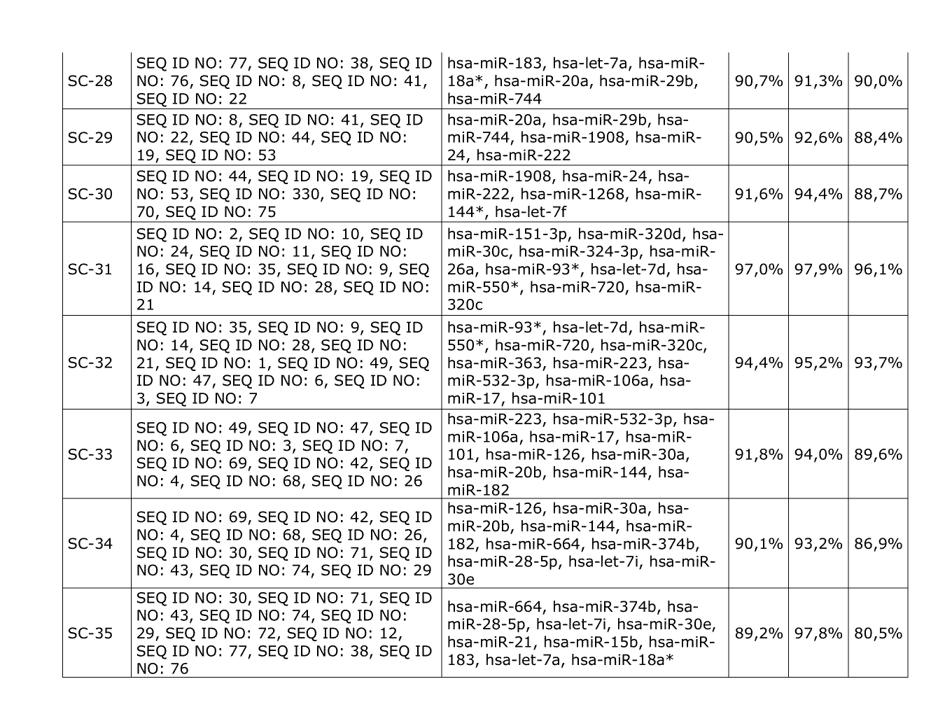

8. The polynucleotide(s) of claim 1, wherein the nucleotide sequences of said miRNAs are the nucleotide sequences according to Set No. 1 to 42 of FIG. 15.

9-22. (canceled)

23. The polynucleotide(s) of claim 1, wherein
(i) the polynucleotide is complementary to the miRNA according to claim 1, or the polynucleotides are complementary to the miRNAs according to claim 1,
(ii) the polynucleotide is a fragment of the polynucleotide according to (i), or the polynucleotides are fragments of the polynucleotides according to (i), or
(iii) the polynucleotide has at least 80% sequence identity to the polynucleotide sequence of the polynucleotide according to (i) or polynucleotide fragment according to (ii), or the polynucleotides have at least 80% sequence identity to the polynucleotide sequences of the polynucleotides according to (i) or polynucleotide fragments according to (ii).

24. A method for diagnosing and/or prognosing of sarcoidosis, comprising the steps of:
determining an expression profile of one or more miRNA(s) in a blood sample from a patient, wherein the nucleotide sequence of said miRNA or the nucleotide sequences of said miRNAs is (are) selected from the group consisting of SEQ ID NOs: 1 to 231 (lists 1, 2, 4 and 7 of FIG. 18), a fragment thereof, and a sequence having at least 80% sequence identity thereto, and
(ii) comparing said expression profile to a reference, wherein the comparison of said expression profile to said reference allows for the diagnosis and/or prognosis of a lung disease, and/or applying an algorithm or a mathematical function to said expression profile, wherein the application of said algorithm or mathematical function to said expression profile allows for the diagnosis and/or prognosis of sarcoidosis.

25. (canceled)

26. The method of claim 24, wherein the nucleotide sequence of said miRNA or the nucleotide sequences of said miRNAs in step (i) is (are) selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (list 1 of FIG. 18), and wherein the comparison and/or application in step (ii) allow(s) for the diagnosis and/or prognosis of sarcoidosis.

27. The method of claim 24, wherein the nucleotide sequence of said miRNA or the nucleotide sequences of said miRNAs in step (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (list 4 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (list 7 of FIG. 18), and wherein the comparison and/or application in step (ii) allow(s) for differential diagnosis with respect to lung cancer and/or COPD.

28. The method of claim 27, wherein the nucleotide sequence of said miRNA or the nucleotide sequences of said miRNAs in step (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (list 2 of FIG. 18), and wherein the comparison and/or application in step (ii) allow(s) for differential diagnosis with respect to lung cancer.

29. The method claim 27, wherein the nucleotide sequence of said miRNA or the nucleotide sequences of said miRNAs in step (i) is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (list 4 of FIG. 18), and wherein the comparison and/or application in step (ii) allow(s) for differential diagnosis with respect to lung cancer and COPD.

30. The method of claim 27, wherein the nucleotide sequence of said miRNA or the nucleotide sequences of said miRNAs in step (i) is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (list 7 of FIG. 18), and wherein the comparison and/or application in step (ii) allow(s) for differential diagnosis with respect to COPD.

31. The method of claim 24, wherein the nucleotide sequences of said miRNAs are the nucleotide sequences according to Set No. 1 to 42 of FIG. 15.

32.-45. (canceled)

46. The method of claim 24, wherein in step (i) one or more polynucleotide(s) according to claim 1 is (are) used for determining an expression profile of one or more miRNA(s) in a blood sample from a patient.

47.-48. (canceled)

49. The method of claim 24, wherein the algorithm or mathematical function is obtained from a reference expression profile of at least one subject with a known clinical condition, and wherein the reference expression profile is the profile of a miRNA that has a nucleotide sequence that corresponds to the nucleotide sequence of the miRNA of step (i), or is the profile of miRNAs that have nucleotide sequences that correspond to the nucleotide sequences of the miRNAs of step (i) and wherein the known clinical condition is selected from the group consisting of healthiness, sarcoidosis, lung cancer, and COPD.

50.-60. (canceled)

61. A kit for diagnosing and/or prognosing of sarcoidosis, comprising
(i) means for determining an expression profile of one or more miRNA(s) in a blood sample from a patient, wherein the nucleotide sequence of said miRNA or the nucleotide sequences of said miRNAs is (are) selected from the group consisting of SEQ ID NOs: 1 to 231 (lists 1, 2, 4 and 7 of FIG. 18), a fragment thereof, and a sequence having at least 80% sequence identity thereto, and
(ii) optionally at least one reference and/or algorithm or mathematical function comprised on at least one data carrier.

62. (canceled)

63. The kit of claim 61, wherein the nucleotide sequence of said miRNA or the nucleotide sequences of said miRNAs determined in (i) is (are) selected from the group consisting of SEQ ID NOs: 34, 36, 39, 40, 50, 56, 57, 58, 61, 62, 66, 79, 80, 81, 83, 84, 85, 89, 90, 93, 94, 96, 98, 99, 100, 101, 102, 104, 113, 128, 129, 132, 133, 142, 143, 144, 148, 151, 155, 156, 163, 166, 169, 170, 171, 173, 179, 193, 195, 196, 199, 200, 201, 203, 206, 214, 216, 221, 223, 225, 226, and 229 (list 1 of FIG. 18).

64. The kit of claim 61, wherein the nucleotide sequence of said miRNA or the nucleotide sequences of said miRNAs determined in (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, 227 (list 2 of FIG. 18), 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, 228 (list 4 of FIG. 18), 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (list 7 of FIG. 18).

65. The kit of claim 64, wherein the nucleotide sequence of said miRNA or the nucleotide sequences of said miRNAs determined in (i) is (are) selected from the group consisting of SEQ ID NOs: 37, 46, 63, 65, 73, 78, 82, 87, 91, 92, 95, 97, 103, 111, 118, 123, 130, 131, 136, 137, 141, 149, 152, 157, 158, 172, 174, 178, 187, 188, 189, 192, 194, 197, 198, 204, 207, 208, 210, 211, 213, 224, and 227 (list 2 of FIG. 18).

66. The kit claim 64, wherein the nucleotide sequence of said miRNA or the nucleotide sequences of said miRNAs determined in (i) is (are) selected from the group consisting of SEQ ID NOs: 1, 2, 3, 4, 5, 6, 7, 8, 9, 10, 11, 12, 13, 14, 15, 18, 19, 20, 21, 22, 23, 24, 25, 26, 27, 28, 29, 30, 32, 33, 38, 41, 42, 43, 44, 45, 47, 51, 54, 55, 64, 68, 69, 70, 71, 72, 74, 75, 76, 77, 105, 106, 107, 108, 109, 110, 112, 114, 115, 116, 120, 121, 125, 126, 134, 138, 145, 153, 154, 159, 161, 162, 164, 165, 167, 168, 175, 177, 183, 184, 185, 186, 190, 191, 209, 218, 219, and 228 (list 4 of FIG. 18).

67. The kit of claim 64, wherein the nucleotide sequence of said miRNA or the nucleotide sequences of said miRNAs determined in (i) is (are) selected from the group consisting of SEQ ID NOs: 16, 17, 31, 35, 48, 49, 52, 53, 59, 60, 67, 86, 88, 117, 119, 122, 124, 127, 135, 139, 140, 146, 147, 150, 160, 176, 180, 181, 182, 202, 205, 212, 215, 217, 220, 222, 230, and 231 (list 7 of FIG. 18).

68. The kit of claim 61, wherein the nucleotide sequences of said miRNAs are the nucleotide sequences according to Set No. 1 to 42 of FIG. 15.

69.-83. (canceled)
